(19) United States
(12) Patent Application Publication
(10) Pub. No.: US 2003/0104987 A1
(43) Pub. Date: Jun. 5, 2003

(54) Methods of using the hyaluronan receptor for endocytosis

(76) Inventors: Paul H. Weigel, Edmond, OK (US); Janet A. Weigel, Edmond, OK (US)

Correspondence Address: Attention: Kathryn L. Hester, Ph.D., Dunlap, Codding & Rogers, P.C., Suite 420, 9400 North Broadway, Oklahoma City, OK, 73114, US

(21) Appl. No.: 10/133,172

(22) Filed: Apr. 25, 2002

Related U.S. Application Data
(63) Continuation-in-part of application No. 09/842,930, filed on Apr. 25, 2001.
(60) Provisional application No. 60/286,468, filed on Apr. 25, 2001.

(51) Int. Cl. A61K 38/17

(52) U.S. Cl. 514/8

(57) ABSTRACT

Methods of using HARE protein or peptide fragments containing at least one of a HA-, a chondroitin- and a chondroitin sulfate-binding domain and monoclonal antibodies raised against HARE that block binding of at least one of HA, chondroitin and chondroitin sulfate thereto. Methods include targeting a compound to a cell expressing HARE or a cell that does not express a functionally active HARE, preventing interaction between a cell expressing HARE and a cell having at least one of a HA coat, a chondroitin coat and a chondroitin sulfate coat, and detecting at least one of HA, chondroitin and chondroitin sulfate in a sample.

### CROSS-REFERENCE TO RELATED APPLICATIONS

This application claims priority under 35 U.S.C. 119(e) of provisional application U.S. Serial No. 60/286,468, filed Apr. 25, 2001, entitled “METHODS OF USING THE HYALURONAN RECEPTOR FOR ENDOCYTOSIS”, the contents of which are hereby expressly incorporated in their entirety by reference.

This application is also a continuation-in-part of U.S. Ser. No. 09/842,930, filed Apr. 25, 2001, entitled “IDENTIFICATION AND USES OF A HYALURONAN RECEPTOR FOR ENDOCYTOSIS”, the contents of which are hereby expressly incorporated in their entirety by reference.

### FEDERALLY SPONSORED RESEARCH OR DEVELOPMENT

The government owns certain rights in the present invention pursuant to a grant from the National Institutes of Health (GM 35978).

### BACKGROUND OF THE INVENTION

1. Field of the Invention

The present invention generally relates to a Hyaluronan (“HA”) Receptor for Endocytosis (HARE) and antibodies against HARE, and more particularly, but not by way of limitation, to methods of targeting compounds to cells and preventing interactions between cells by utilizing HARE and/or such antibodies.

2. Brief Description of the Related Art

HA, also referred to herein as hyaluronic acid, or hyaluronan, is an important and often abundant extracellular matrix component of all tissues, in particular cartilage, skin and vitreous humor (Evered and Whelan, The Biology of Hyaluronan, Ciba Fnd. Symposium, 143:1 (1989)). HA plays a key role in development, morphogenesis and differentiation, in cell adhesion and proliferation, and in inflammation and wound healing (Evered and Whelan, The Biology of Hyaluronan, Ciba Fnd. Symposium, 143:1 (1989); Toole, J. Intern. Med. 242:35 (1997); Knudson and Knudson, FASEB J. 7:1233 (1993); Laurent and Fraser, FASEB J. 6:2397 (1992)). In humans, the total body turnover of HA is several grams per day (Evered and Whelan, The Biology of Hyaluronan, Ciba Fnd. Symposium, 143:1 (1989)). Although local turnover of HA occurs in avascular tissues, particularly cartilage (Hua et al, J. Cell Sci. 106:365 (1993); Aguiar et al, Exp. Cell Res. 252:292 (1999)), two major clearance systems are responsible for HA degradation and removal in the body (Laurent and Fraser, FASEB J. 6:2397 (1992)). The first is the lymphatic system, which accounts for about 85% of the HA turnover, and the second is in the liver, which accounts for the other approximately 15% of the total body HA turnover.

Throughout the body, HA is continuously synthesized and degraded in almost all tissues. At the same time, chondroitin sulfate and other glycosaminoglycans are also released from the cleavage of proteoglycans, especially aggregating proteoglycans associated with HA. Large native HA molecules (about 10⁷ Da) are partially degraded into large fragments (about 10⁶ Da) that are released from the matrix and enter the lymphatic system, thereafter flowing to lymph nodes.

The lymph nodes completely degrade the majority of HA (about 85%) by currently unknown mechanisms. Neither the responsible cell type, the receptor involved, nor the location in lymph nodes at which HA uptake and degradation occurs has been determined. The remaining HA (about 15%) that escapes degradation in the lymph nodes ultimately flows into the blood at the thoracic duct. Since HA is an exceptionally viscous polysaccharide in solution, it would be deleterious for the blood concentration of HA, even at relatively low molecular weight, to increase. Clearance of this circulating HA and the other glycosaminoglycan degradation fragments, such as chondroitin sulfate, is important for normal health (Evered and Whelan, The Biology of Hyaluronan, Ciba Fnd. Symposium, 143:1 (1989); Laurent and Fraser, FASEB J. 6:2397 (1992)). For example, elevated serum HA levels are associated with a variety of diseases and pathological conditions such as liver cirrhosis, rheumatoid arthritis, psoriasis, scleroderma, fibromyalgia and some cancers (Yamad et al, Acta Haematol. 99:212 (1998); Lai et al, J. Lab Clin. Med. 131:354 (1998); Yaron et al, J. Rheumatol. 24:2221 (1997)).

Liver endothelial cells (LECs) in vertebrate liver express a very active, recycling endocytic receptor that removes these extracellular matrix-derived fragments of HA and other glycosaminoglycans, including chondroitin sulfate, from the blood (Laurent and Fraser, FASEB J. 6:2397 (1992); DeBleser et al, Gut, 35:1509 (1994); Raja et al, J. Biol. Chem. 263:16661 (1988); McGary et al, Biochem. J. 257:875 (1989); McGary et al, Hepatology, 18:1465 (1993)). ICAM-1, a 90 kDa protein also known as CD54 (Hayflick et al, Immunol. Res. 17:313 (1998)), was previously misidentified as the HA Receptor (HAR) on LECs (Forsberg and Gustafson, Biochim. Biophys. Acta, 1078:12 (1991); McCourt et al, J. Biol. Chem. 269:30081 (1994)). This research attempted to purify the HA receptor without the use of an assay to measure HA-binding activity. The claim that the HA Receptor on LECs had been purified was subsequently acknowledged to be an artifact due to the nonspecific binding of ICAM-1 to the HA affinity resin (McCourt and Gustafson, Int. J. Biochem. Cell Biol. 29:1179 (1997); McCourt et al, Hepatology 30:1276 (1999)). In any case, since ICAM-1 is not a coated pit-targeted endocytic receptor, it is not the true HA receptor in LECs.

In addition to the normal turnover of HA in tissues throughout the body, a wide range of biomedical and clinical applications use exogenous HA that is also removed from the lymphatics or ultimately from the blood and degraded by the LEC HARE. For example, HA is used extensively in eye surgery, in the treatment of joint diseases including osteoarthritis, and is being developed as a drug delivery vehicle. Numerous studies have explored the benefit of HA during wound healing. The exogenous HA introduced in these various applications is naturally degraded by the lymph and LEC systems noted above.

In two previous studies, one using a photoaffinity derivative of HA (Yannariello-Brown et al, J. Biol. Chem. 267:20451 (1992)) and the other using a novel ligand blot assay with ¹²⁵I-HA (Yannariello-Brown et al, Glycobiol. 7:15 (1997)), two specific HA-binding proteins in isolated rat LECs were identified at about 175 kDa and about 300 kDa.

Therefore, there exists a need in the art for identification and isolation of a HA receptor for endocytosis (HARE), as well as antibodies directed thereto, and methods of targeting compounds to cells and preventing interactions between cells by utilizing HARE and/or such antibodies.

### SUMMARY OF THE INVENTION

The present invention is related to methods of using HA, HARE and/or a monoclonal antibody raised against an HA-binding domain of HARE to target compounds to specific cells or to prevent interactions between two types of cells.

In one embodiment, the present invention relates to a method of targeting a compound to a tissue of an individual wherein cells of the tissue express a functionally active HARE. The compound is conjugated to at least one of HA, chondroitin, chondroitin sulfate, and a monoclonal antibody that selectively binds to an epitope of HARE. An effective amount of the complex formed of compound conjugated to HA-, chondroitin-, chondroitin sulfate-, or HARE monoclonal antibody can then be administered to the individual. The compound may be, for example, a chemotherapeutic agent or a radioisotope, or the compound may be deleterious to cells in close proximity to the cells expressing HARE on a surface thereof upon delivery of the compound to the cells expressing HARE.

In another embodiment, the present invention relates to a method of preventing interaction between a cell expressing HARE on a surface thereof and a cell having at least one of an HA coat, a chondroitin coat and a chondroitin sulfate coat. An effective amount of a compound that inhibits binding of at least one of HA, chondroitin and chondroitin sulfate to HARE, such as a mimetic peptide or a monoclonal antibody that selectively binds to an epitope of HARE and inhibits binding of at least one of HA, chondroitin and chondroitin sulfate to HARE, is administered to prevent such interaction.

In yet another embodiment, the present invention includes a method of targeting a compound to a cell of an individual wherein the cell does not express a functionally active HARE on a surface thereof by administering an effective amount of a monoclonal antibody that binds HARE and blocks binding of at least one of HA, chondroitin and chondroitin sulfate to the HARE. The compound can then be conjugated to at least one of HA, chondroitin and chondroitin sulfate, and an effective amount of the conjugate can be administered to the individual such that the compound is targeted to a cell that expresses at least one cell surface or extracellular matrix component capable of binding at least one of HA, chondroitin and chondroitin sulfate.

In yet another embodiment of the present invention, methods of detecting at least one of HA, chondroitin and chondroitin sulfate in a sample, as well as quantitating the presence of each of HA, chondroitin and chondroitin sulfate, are provided. A HARE protein or peptide fragment containing at least one of an HA-, a chondroitin-, and a chondroitin sulfate-binding domain is provided and may be immobilized on a solid support. The sample is then contacted with the HARE protein or peptide fragment to form a mixture, whereby at least one of HA, chondroitin and chondroitin sulfate present in the sample binds to the HARE protein or peptide fragment. Unbound sample is then washed away, and the HA, chondroitin or chondroitin sulfate bound to the HARE protein or peptide fragment may be detected by one of two ways. First, at least one of labeled HA, labeled chondroitin and labeled chondroitin sulfate is contacted with the mixture, and a determination that at least one of HA, chondroitin and chondroitin sulfate is present in the sample is made if the labeled HA, chondroitin or chondroitin sulfate does not bind or has decreased binding to the HARE protein or peptide fragment. Second, a labeled HARE protein or peptide fragment containing at least one of an HA-, chondroitin- and chondroitin sulfate-binding domain is contacted with the mixture. If at least one of HA, chondroitin and chondroitin sulfate is present in the sample and bound to the immobilized HARE protein or peptide fragment, the labeled HARE protein or peptide fragment will bind thereto, and therefore can be detected by the presence of labeled HARE protein or peptide fragment on the immobilized HARE protein or peptide fragment.

In yet another embodiment, the present invention includes a method of treating an individual having an elevated level of at least one of HA, chondroitin and chondroitin sulfate in the blood or lymph by administering an effective amount of a vector encoding a functionally active HARE protein or a “HARE-like” protein. A “HARE-like” protein comprises a LINK domain and at least one motif selected from the group consisting of SEQ ID NOS:6-18 and sequences that are substantially identical to or only have conserved or semi-conserved amino acid substitutions to SEQ ID NOS:6-18, and is able to bind to and endocytose at least one of HA, chondroitin and chondroitin sulfate.

Other objects, features and advantages of the present invention will become apparent from the following detailed description when read in conjunction with the accompanying figures and appended claims.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1. Model for the organization of the two rat liver HARE isoreceptors. HARE preparations may contain two independent HARE isoreceptors or may be a super-large complex composed of two (or three) copies of the 175 HARE protein and one copy of the 300 kDa HARE complex. The 300 kDa HARE is a heterotrimeric complex of three subunits (α, β and γ) that are disulfide bonded.

FIG. 2. Nucleic acid (SEQ ID NO:1) and deduced amino acid (SEQ ID NO:2) sequences of the 4.7-kb cDNA encoding the rat 175-kDa HARE. The artificial cDNA containing 4708 nucleotides encodes a 1431 amino acid recombinant 175-kDa HARE protein, whose deduced amino acid sequence begins with a serine. Amino acid sequences verified by peptide sequence analysis of the purified HARE are underlined, and the two N-terminal peptides found in the purified protein are underlined and in italics. Putative N-glycosylation sites are in boldface, and Cys residues are highlighted in boldface and italics. Three alternative N-glycosylation sites of the type -N-X-C- are located at N¹³⁵, N²¹⁸ and N⁹³⁰. The predicted transmembrane domain of the type I membrane protein is underlined and in boldface. The three shaded regions in the cytoplasmic domain are potential motifs for targeting the receptor to clathrin-coated pits. Potential HA-binding motifs of the type B-X₇-B, which are in the predicted extracellular domain, are enclosed in boldface [brackets].

FIG. 3. Domain structure of the 175 kDa rat HARE protein. The scheme depicts the organization of multiple protein domains within the 1431 amino acid HARE protein that are identified by numerous predictive search programs such as SMART, CD-Search, and other sites linked to ExPASy or NCBI. TM indicates the transmembrane domain; E2, Ea and Ec represent, respectively EGF-2, lamin-like EGF and EGF-Ca⁺² domains; potential N-linked glycosylation sites are indicated by the Y symbols.

FIG. 4A. Reactivity of a panel of 175 HARE-mAbs in Western analysis after nonreducing SDS-PAGE of LEC extracts. Ascites from 11 hybridoma clones that were positive in ELISA screens with the 175 HARE antigen were screened (at a 1:1,000 dilution) for reactivity with lysates of rat LECs. Seven of these clones showed strong reactivity with proteins at both 175 and 300 kDa (lanes 1-8 except lane 3). Clone 54 only recognizes the reduced protein (FIG. 4B). Three clones gave very different patterns (lanes 9-11) and do not recognize the 175 HARE antigen. R and N show mouse antisera raised against reduced (R) or nonreduced (N) 175 HARE antigen. The solid and open arrows indicate the positions of the 300 HARE and 175 HARE, respectively.

FIG. 4B. Reactivity of a panel of anti-175 HARE mAbs in Western analysis after reducing SDS-PAGE of LEC extracts. Only mAbs 54 (lane 3) and 159 (lane 5) show strong reactivity which is identical with the reduced 175 HARE and 300 HARE proteins. The solid and open arrows indicate the positions of the nonreduced 300 HARE and 175 HARE, respectively. MAb-174, which also blocks HA binding (FIGS. 5 and 6), shows weaker reactivity with the reduced 175 HARE and the 260 kDa subunit of the 300 HARE (lane 6). The other mAbs, including those positive for the nonreduced proteins, are not reactive.

FIG. 5. Antibody inhibition of HA endocytosis by HARE in LECs. Cultured primary rat LECs were washed and incubated for 60 min at 37° C. with 2 μg/ml ¹²⁵I-HA in MEM medium containing 0-9 μg/ml of IgG (affinity purified from ascites fluid using Protein G-Sepharose, or rabbit anti-mouse IgM-Sepharose in the case of #159) from each of five different hybridomas against the 175 HARE. The plates were then chilled on ice, the media was aspirated, the wells were washed 3 times and the cells were solubilized in 0.3 N NaOH. Radioactivity and protein content were determined for each of the samples. The mean of triplicates ±SD are expressed as percent of control (dpm/mg protein).

FIG. 6. Specific monoclonal antibodies against HARE inhibit HA endocytosis in SK-Hep1 transfectants expressing the 175 kDa HARE. The indicated SK-Hep1 clones expressing the 175 kDa HARE were allowed to internalize ¹²⁵I-HA as described above with no addition or in the presence of either mAb-174 or mAb-235 as indicated.

FIG. 7. Alignment of the rat 175 kDa HARE deduced amino acid sequence with a family of hypothetical protein sequences of unknown function. Sequences were aligned with DNAs is (Version 2.50), saved as a text file and edited in Microsoft Word. The hypothetical protein sequences, all of which are human, are designated by their GenBank protein accession numbers. Our deposited sequences for the rat 175 kDa HARE (rHARE) are under accession numbers AY007370 and AAG13634 for the nucleic acid (SEQ ID NO:1) and protein (SEQ ID NO:2) sequences, respectively. The recombinant 175 kDa HARE that was constructed in order to demonstrate the functionality of this receptor starts with serine (arrow). Residues in HARE identical to two or more of the other sequences are shaded in yellow. Conserved cysteine residues are in boldface and shaded red. The residues under the solid bold line are identified as an extracellular Link domain (XLink), a putative HA-binding domain. The dashed line is above the approximate boundaries of a single putative transmembrane domain in each protein. Regions within boxes denote candidate φXXB motifs for targeting to coated pits.

FIG. 8. Immunocytochemical localization of HARE in human liver, spleen and lymph node. Sections of human spleen (A and B), lymph node (C) and liver (D) were treated with either anti-HARE mAb-30 (A, C and D) or mouse serum (B) and then stained. A relatively low magnification is shown (the bar represents ˜500 μm) to emphasize the localization of the human HARE protein in the sinusoidal regions of each tissue.

FIG. 9A. Nucleic acid (SEQ ID NO:3) and deduced protein (SEQ ID NO:4) sequences of the human 190 kDa HARE. The HARE nucleotide sequence was assembled based on the sequences of BAB15793 and specific RT-PCR products derived from human spleen (as described in detail previously in U.S. Ser. No. 09/842,930). The solid bars underline 17 consensus N-glycosylation sites. The arrow indicates a nucleotide sequence error in BAB15793 (omission of an A, in boldface) that results in a frame-shift, which adds 210 amino acids (in italics) and deletes eight at the N-terminal end of the ORF derived from BAB15793. A second error in the BAB15793 nucleotide sequence at T¹³⁸⁶ (rather than C) and noted in boldface is silent. Amino acid sequences within solid or dashed boxes indicate the peptides of the authentic human 190 kDa HARE (immunoaffinity purified from human spleen) that were identified, respectively, by direct sequencing or by molecular mass analysis (as described in detail previously in U.S. Ser. No. 09/842,930). Human spleen HARE amino acid sequences that were not in the BAB15793 protein sequence but were confirmed in RT-PCR products are boxed and underlined.

FIG. 9B. Nucleotide (SEQ ID NO:19) and amino acid (SEQ ID NO:20) sequence for the partial human 190 kDa HARE cDNA including 237 residues encoded by the sequence upstream of the likely start site for the 190 kDa HARE. Note that the numbering is different than for the sequence given for the 190 kDa HARE in FIG. 9A. The 237 residues encoded by the sequence upstream of the likely start site for the 190 kDa are in boldface & italics.

FIG. 10. Domain organization of the human 190 kDa HARE. The scheme depicts the organization of protein domains identified by the programs Pfam-HMM, CD-Search, ScanProsite or SMART (Schultz et al, Proc. Natl. Acad. Sci. USA, 95:5857 (1998)). Abbreviations used for some of the domains include CD (cytoplasmic domain), TMD (transmembrane domain), M-T (metallothionein), and EGF-C, EGF-L or EGF-2 for epidermal growth factor calcium, laminin or type 2 domains, respectively.

FIG. 11. Sequence alignment of the human (SEQ ID NO:4) and rat (SEQ ID NO:2) HARE proteins. Sequences for the two smaller HARE proteins were aligned using SIM (at www.ExPAS , and as described in detail in U.S. Ser. No. 09/842,930) and then saved as a Microsoft Word file for highlighting and annotation. Identical residues found in both sequences are shaded in yellow. Conserved consensus N-linked glycosylation sides are in boldface and highlighted in gray. Solid black bars indicate potential -N-X-Cys-glycosylation sites, two of which are conserved. Cysteine residues are boldface and shaded red where identical between the two proteins. The arrow denotes the beginning of the least conserved regions of the two proteins: their cytoplasmic domains. The residues under the solid blue line are identified as an extracellular Link domain (XLink), a putative hyaluronan-binding domain. The residues under the dashed blue line indicate the single predicted transmembrane domain. The three conserved candidate φXXB motifs are within the two blue boxes. Ser, Thr or Tyr residues that are predicted (by NetPhos 2.0; Blom et al, J. Molec. Biol. 294:1351 (1999)) to be phosphorylated are shown in boldface white with red highlighting.

FIG. 12. Model for the organization of the two human spleen HARE isoreceptors. The 190 kDa and ˜315 kDa HARE isoreceptors isolated from human spleen are depicted as separate species in approximate molar ratios of 1:2, respectively. The 190 kDa HARE contains only one protein. The large HARE complex is composed of two (or perhaps three) disulfide-bonded subunits of about 250 kDa and one subunit of 220 kDa, respectively. Preliminary results indicate that the molar ratios of the affinity purified 190 kDa and ˜315 kDa HARE isoreceptors from different tissues may be different. All HARE proteins and subunits are membrane-bound and are predicted to contain small cytoplasmic domains and very large ectodomains. The HARE proteins are elongated, rather than globular (Yannariello-Brown et al, Glycobiol. 7:15 (1997)).

FIG. 13. Scheme for HA turnover and metabolism in humans. The scheme depicts the overall turnover of HA present initially in the ECM of tissues throughout the body. Partially degraded HA is flushed from the ECM into lymph by the flow of fluid through the tissue. Some HA may be degraded locally in the tissue, but most HA (˜85%) is delivered to and removed by lymph nodes. The remaining HA (˜15%) enters the blood, and the majority thereof is cleared by the liver, while the spleen also removes a small fraction. HARE, which is expressed on the surface of sinusoidal endothelial cells of lymph node and liver, binds the circulating HA and removes it from the lymph or blood by internalization through the clathrin coated pit endocytic pathway. The average size and concentration of the HA decreases in going from ECM to lymph node to blood (Laurent and Fraser, FASEB J. 6:2397 (1992); Laurent and Fraser, Degradation of Bioactive Substances: Physiology and Pathophysiology, 249, CRC Press, Boca Raton, Fla. (1991); Tengblad et al, Biochem. J. 236:521 (1986)).

FIG. 14. Chondroitin Sulfate-A or HA compete for HA endocytosis by cells expressing rHARE. Two independent SK-HARE clones (#26 and #36) are shown. The accumulation of ¹²⁵I-HA was measured in a similar manner to that described above in relation to FIGS. 5 and 6.

FIG. 15. Keratin Sulfate or Heparan Sulfate do not compete for HA endocytosis by cells expressing rHARE. Two independent SK-HARE clones (#26 and #36) are shown. The accumulation of ¹²⁵I-HA was measured in a similar manner to that described above in relation to FIGS. 5 and 6.

FIG. 16. Chondroitin Sulfate-D and HA compete differentially for HA binding at 4° C. versus endocytosis at 37° C. by cells expressing rHARE.

FIG. 17. Effect of various glycosaminoglycans on binding (at 4° C.) or endocytosis (at 37° C.) of HA by cells expressing rHARE.

FIG. 18. Effect of various glycosaminoglycans on binding of HA at 4° C. or endocytosis of HA at 37° C. by cells expressing rHARE.

FIG. 19. HARE is present in normal human bone marrow. Sections of normal human bone marrow were treated with either anti-HARE mAb-30 (upper panels and lower left panel) or mouse serum (lower right panel) and then stained.

FIG. 20. HARE is absent in a human bone marrow metastasis but is increased at the interface between cancer and normal marrow. Sections of human bone marrow metastasis were treated with either anti-HARE mAb-30 (upper right panel and lower panels) or mouse serum (upper left panel) and then stained. The tumor is to the upper left in all four panels.

FIG. 21. HARE is absent in a human bone marrow metastasis but present in normal marrow. Sections of normal human bone marrow (lower panel) and human bone marrow metastasis (upper panel) treated with anti-HARE mAb-30 from FIGS. 19 and 20 are shown at higher magnification.

FIG. 22. Carcinoma cells express cell surface HA. MDA-MB-231 (A) and PC3 (B) cells express cell surface HA as demonstrated by their staining with peroxidase following binding of a biotinylated HA binding protein. MDA-MB-435 (C) and DU145 (D) cells show virtually no cell surface HA. This staining is specific for HA on the tumor cell surface, since it is virtually abolished (inserts) by pretreatment with the very specific hyaluronidase from Streptomyces.

FIG. 23. MDA-MB-231 and PC3 cells express a cell surface coat of HA. MDA-MB-231 (A) and PC3 (B) cells express cell surface HA coats as demonstrated by the particle exclusion assay. MDA-MB-435 cells (C) or DU145 cells (not shown) show virtually no cell surface HA. This exclusion zone is due to HA on the tumor cell surface and is abolished by pretreating these cells with Streptomyces hyaluronidase (inserts).

FIG. 24. SK-HARE cells express functionally active HARE capable of endocytosing fluorescent-HA. The accumulation of fluorescent-HA into endocytic vesicles by SK-HARE cells (A) is inhibited by a 50-fold excess nonlabeled HA (B). Similarly treated parental SK-Hep1 cells show no ability to bind and internalize significant amounts of the fluorescent hyaluronan without (C) or with (D) excess HA.

FIG. 25. Aggregation of carcinoma cells with SK-HARE or SK-Hep1 cells. MDA-MB-231, and PC3 cells show increased aggregation with SK-HARE cells compared to SK-Hep1 control cells. MDA-MB-435 and DU145 cells having little surface HA show decreased ability to aggregate with SK-HARE cells (top panel). Aggregation of carcinoma cells and SK-HARE cells could be specifically blocked by addition of free competing HA (middle panel) or hyaluronidase treatment of carcinoma cells (bottom panel).

FIG. 26. Human breast carcinoma that metastasized to lymph node expresses cell surface HA and is at sites of HARE Expression. Human metastatic breast carcinoma cells express cell surface HA, as demonstrated by staining with the biotinylated HA binding protein without (A) and with (B) hyaluronidase treatment. The carcinoma cells have arrested in axillary lymph nodes at sites of HARE expression (C). A negative control treated with non-immune IgG is shown in D.

FIG. 27. Perfusion of isolated rat liver with ¹²⁵I-HA. The presence of unlabeled HA inhibits ¹²⁵I-HA clearance by intact liver.

FIG. 28. Perfusion of isolated rat liver with ¹²⁵I-HA. The anti-HARE blocking antibody mAB-174 specifically inhibits HA clearance by intact liver. Mouse IgG, used as a control, had essentially no effect on HA clearance (compare to “No addition” in FIG. 27).

FIG. 29. Perfusion of isolated rat liver with ¹²⁵I-HA. The anti-HARE blocking antibody mAb-174 specifically inhibits HA degradation by intact liver.

FIG. 30. Methods of targeting a compound to or preventing interaction with a cell expressing HARE.

FIG. 31. An in-frame region upstream of the 175-kDa cDNA encodes amino acid sequences present in the larger HARE subunits. LECs were lysed in Laemmli (1970) buffer containing 5% beta-mercaptoethanol and samples were subjected to SDS-PAGE and electrotransfer. Nitrocellulose strips were cut and incubated with: lane 1, a mixture of 8 mAbs that recognize all three HARE proteins (i.e. the 175-kDa HARE and the 260-kDa and 230-kDa subunits of the 300 HARE complex); lane 2, pre-immune goat IgG; lane 3, goat IgG (Ab2 in the diagram) raised against a 16-amino acid putative coding region (TVLVPSRRAFEDMDQNK⁻⁹¹) upstream of the amino terminal start of the purified rat 175-kDa protein; lane 4, preimmune sheep IgG; lane 5, sheep IgG (Ab1 in the diagram) raised against a peptide corresponding to the sequence PKCPLKSKGVKK⁷⁷³ within the rat 175-kDa protein. Strips were washed, incubated with the appropriate secondary antibody-alkaline phosphatase conjugates and substrates for color development.

FIG. 32. The core proteins of the human 190 kDa HARE and the rat 175 kDa HARE are essentially the same size after removal of N-linked oligosaccharides. Purified rat and human HARE (1 mg) were denatured by boiling in 0.5% SDS, mixed with 0.5% NP-40 and de-N-glycosylated by treatment with N-glycosidase F at 37° C. overnight as described by the manufacturer. After SDS-PAGE and electro-transfer to nitrocellulose, the HARE protein bands were detected using anti-HARE mAbs against the rat 175 kDa HARE. The position of the rat 175 kDa HARE and the human 190 kDa HARE are indicated by the solid arrows. After removal of the N-linked oligosaccharides, both core proteins migrate at the same position, marked by the dashed arrow, indicating that both proteins are essentially identical in size. The apparently larger size of the human 190 kDa HARE relative to the rat HARE is due to the presence of either more or larger oligosaccharides.

FIG. 33. Comparison of HA binding by the native and recombinant 175-kDa HARE proteins. Membranes from isolated LECs (lanes 1 and 2) and SK-175 HARE-34 cells (lanes 3 and 4) were solubilized in TBS containing 0.5% NP40 plus protease inhibitors, and HARE proteins were immunoprecipitated using mAb-30 coupled to Sepharose. The proteins were eluted with sample buffer, subjected to SDS-PAGE and electrotransfer, and the nitrocellulose was incubated overnight in TBS containing 0.5% Tween-20. Ligand blotting with 1 μg/ml ¹²⁵I-HA (lanes 1 and 3 from autoradiogram) was performed as described previously in U.S. Ser. No. 09/842,930. The same blots were then incubated in TBS containing 1% BSA and subjected to Western analysis (lanes 2 and 4) using a mixture of eight mAbs against HARE. A series of dilutions verified that the Western staining responses for both samples were proportional to protein load and were not saturated. The open and solid arrows indicate, respectively, the ˜300-kDa and 175-kDa HARE species. The HA-binding intensity relative to the Western staining of the 175-kDa HARE was essentially the same from LECs and the stable cells.

FIG. 34. Cell surface expression of the recombinant 175-kDa HARE in stably transfected cells. After blocking nonspecific binding sites, SK-175 HARE cells or SK-Hep-1 cells transfected with vector alone were incubated, as indicated, with either nothing, 1 μg/ml mAb-30, 1 μg/ml mouse IgG or a mixture of four mAbs (#s 30, 154, 174 and 235 each at 1 μg/ml). The cells were washed, incubated with Alexa 488-conjugated secondary antibody for 45 min on ice and processed for FACS analysis.

FIG. 35. FACS analysis of fl-HA uptake in SK-175 HARE cells mediated by the 175-kDa HARE. SK-Hep-1 cells transfected with vector alone (panel A) or SK-175 HARE-34 cells (panels B and C) were grown to confluence in 6-well tissue culture plates, washed and preincubated at 37° C., as indicated in the figure, with no addition or nonlabeled HA (panel B) or mouse IgG or mAb-174 (panel C) followed by fl-HA. The same five conditions were used in panel A.

FIG. 36. Confocal microscopy of the 175-kDa HARE in SK-175 HARE cells. The cellular distributions of the recombinant HARE, fl-HA, clathrin and lysosomes were determined in SK-175 HARE-34 cells. Panels A-C show the co-localization of clathrin (A) and HARE (B) in the overlay picture (C). The different distribution patterns of HARE (D) and Lysotracker (E) in cells incubated with unlabeled HA are shown in the overlay picture (F). Panel I shows the co-localization pattern of fl-HA (G) and Lysotracker (H). The effect of excess unlabeled HA on the uptake of fl-HA is shown in panel ]. The background staining of SK-175 HARE cells with rabbit IgG is shown in Panel K. Panel L shows the anti-HARE staining of SK-Hep-1 cells stably transfected with the backbone plasmid (containing no cDNA insert). The bar in A (20 μm) applies to panel A-C and the bar in D (50 μm) applies to panels D-L.

### DETAILED DESCRIPTION OF THE INVENTION

Before explaining at least one embodiment of the invention in detail, it is to be understood that the invention is not limited in its application to the details of construction and the arrangement of the components or steps or methodologies set forth in the following description or illustrated in the drawings. The invention is capable of other embodiments or of being practiced or carried out in various ways. Also, it is to be understood that the phraseology and terminology employed herein is for the purpose of description and should not be regarded as limiting.

The term “functionally active HARE” as used herein will be understood to include a protein or peptide which is able to specifically bind at least one of HA, chondroitin and chondroitin sulfate, and when present on a surface of a cell, is able to endocytose the bound HA, chondroitin or chondroitin sulfate. The term “active peptide fragment of HARE” as used herein will be understood to include polypeptides which are able to specifically bind at least one of HA, chondroitin and chondroitin sulfate. Such active peptide fragments of HARE may include soluble fragments of HARE. One of ordinary skill in the art, given this Specification containing descriptions of the cytoplasmic, transmembrane and extracellular domains of HARE (as discussed in more detail herein below in the Example), should be able to identify and select portions of the HARE protein (e.g., the extracellular domain of HARE or portions thereof, such as an HA-binding domain of HARE) which retain the ability to bind at least one of HA, chondroitin and chondroitin sulfate.

In addition, the present invention also includes “HARE-like” proteins that are able to specifically bind at least one of HA, chondroitin and chondroitin sulfate. When the “HARE-like” proteins are present on a surface of a cell, the “HARE-like proteins” may further be able to endocytose the bound HA, chrondroitin and/or chondroitin sulfate. Such “HARE-like” proteins contain a LINK domain (as discussed in further detail herein after) and at least one other motif as defined in Table III.

As used herein, the terms “nucleic acid segment”, “DNA sequence”, “DNA segment” and “nucleic acid sequences” are used interchangeably and refer to a DNA molecule which has been isolated free of total genomic DNA of a particular species. Therefore, a “purified” DNA or nucleic acid segment as used herein refers to a DNA segment which contains a HA Receptor for Endocytosis (“HARE”) coding sequence or fragment thereof yet is isolated away from, or purified free from, unrelated genomic DNA, for example, mammalian host genomic DNA. Included within the term “DNA segment”, are DNA segments and smaller fragments of such segments, and also recombinant vectors, including, for example, plasmids, cosmids, phage, viruses, and the like.

Similarly, a DNA segment comprising an isolated or purified HARE gene refers to a DNA segment including HARE coding sequences isolated substantially away from other naturally occurring genes or protein encoding sequences. In this respect, the term “gene” is used for simplicity to refer to a functional protein, polypeptide or peptide encoding unit. As will be understood by those skilled in the art, this functional term includes genomic sequences, cDNA sequences or combinations thereof. “Isolated substantially away from other coding sequences” means that the gene of interest, in this case HARE or a fragment thereof, forms the significant part of the coding region of the DNA segment, and that the DNA segment does not contain large portions of naturally-occurring coding DNA, such as large chromosomal fragments or other functional genes or DNA coding regions. Of course, this refers to the DNA segment as originally isolated, and does not exclude genes or coding regions later added to, or intentionally left in the segment by the hand of man.

Preferably, DNA sequences in accordance with the present invention will further include genetic control regions which allow for the expression of the sequence in a selected recombinant host. Of course, the nature of the control region employed will generally vary depending on the particular use (e.g., cloning host) envisioned. One of ordinary skill in the art, given this Specification, would be able to identify and select genetic control regions which can be utilized in accordance with the present invention to enhance expression of a HARE gene. Examples of specific genetic control regions which may be utilized are described in more detail herein below with regard to specific recombinant host cells.

In particular embodiments, the invention concerns the use of isolated DNA segments and recombinant vectors incorporating DNA sequences which encode a HARE gene or a fragment thereof, that includes within its amino acid sequence an amino acid sequence in accordance with at least a portion of SEQ ID NO:2, SEQ ID NO:4 or SEQ ID NO:20. Moreover, in other particular embodiments, the invention concerns isolated DNA segments and recombinant vectors incorporating DNA sequences which encode a gene that includes within its DNA sequence the DNA sequence of a HARE gene or DNA or fragment thereof, and in particular to a HARE gene or cDNA or fragment thereof, corresponding to rat or human HARE. For example, where the DNA segment or vector encodes a full length HARE protein, or is intended for use in expressing the HARE protein, preferred sequences are those which are essentially as set forth in SEQ ID NO:2, SEQ ID NO:4 or SEQ ID NO:20. In an alternative embodiment, where the DNA segment may encode a functional portion of the HARE protein, such as a soluble form of the protein which still retains the ability to bind at least one of HA, chondroitin and chondroitin sulfate, for example a peptide containing an extracellular domain of HARE or an HA-binding domain of HARE, preferred sequences are at least a portion of those which are essentially as set forth in SEQ ID NO:2, SEQ ID NO:4 or SEQ ID NO:20. It is within the abilities of one of ordinary skill in the art, given this Specification, to identify the DNA segments encoding the cytoplasmic, transmembrane and extracellular domains of the HARE protein and to locate and select the portions of the amino acid sequences of SEQ ID NO:2, SEQ ID NO:4 or SEQ ID NO:20 which encode the extracellular domain of HARE, or a portion thereof, and not the cytoplasmic or transmembrane domain of HARE.

Nucleic acid segments having functional HARE activity may be isolated by the methods described herein. The term “a sequence essentially as set forth in SEQ ID NO:2”, “a sequence essentially as set forth in SEQ ID NO:4” or “a sequence essentially as set forth in SEQ ID NO:20” means that the sequence substantially corresponds to at least a portion of SEQ ID NO:2, SEQ ID NO:4 or SEQ ID NO:20, respectively, and has relatively few amino acids which are not identical to, or a biologically functional equivalent of, the amino acids of SEQ ID NO:2, SEQ ID NO:4 or SEQ ID NO:20, respectively. The term “biologically functional equivalent” is well understood in the art and is further defined in detail herein as a gene having a sequence essentially as set forth in SEQ ID NO:2, SEQ ID NO:4 or SEQ ID NO:20, and that is associated with the ability to bind and endocytose at least one of HA, chondroitin and chondroitin sulfate.

One of ordinary skill in the art would appreciate that a nucleic acid segment encoding a functionally active HARE may contain conserved or semi-conserved amino acid substitutions to the sequences set forth in SEQ ID NO:2, SEQ ID NO:4 and SEQ ID NO:20 and yet still be within the scope of the invention.

In particular, the art is replete with examples of practitioner's ability to make structural changes to a nucleic acid segment (i.e. encoding conserved or semi-conserved amino acid substitutions) and still preserve its enzymatic or functional activity. See for example: (1) Risler et al. “Amino Acid Substitutions in Structurally Related Proteins. A Pattern Recognition Approach.” J. Mol. Biol. 204:1019-1029 (1988) [“ . . . according to the observed exchangeability of amino acid side chains, only four groups could be delineated; (i) Ile and Val; (ii) Leu and Met, (iii) Lys, Arg, and Gin, and (iv) Tyr and Phe.”]; (2) Niefind et al. “Amino Acid Similarity Coefficients for Protein Modeling and Sequence Alignment Derived from Main-Chain Folding Anoles.” J. Mol. Biol. 219:481-497 (1991) [similarity parameters allow amino acid substitutions to be designed]; and (3) Overington et al. “Environment-Specific Amino Acid Substitution Tables: Tertiary Templates and Prediction of Protein Folds,” Protein Science 1: 216-226 (1992) [“Analysis of the pattern of observed substitutions as a function of local environment shows that there are distinct patterns . . . ” Compatible changes can be made.], the contents of all of which are hereby expressly incorporated herein by reference. Standardized and accepted functionally equivalent amino acid substitutions are presented in Table I.

These references and countless others indicate that one of ordinary skill in the art, given a nucleic acid sequence, could make substitutions and changes to the nucleic acid sequence without changing its functionality. Also, a substituted nucleic acid segment may be highly similar and retain its functional activity with regard to its unadulterated parent, and yet still fail to hybridize thereto under standard stringent hybridization conditions. However, while hybridization may not occur at such stringent hybridization conditions, hybridization may be observed at less stringent, relaxed hybridization conditions. Stringent and relaxed hybridization conditions are discussed in more detail herein below.

TABLE I

| | Conservative and Semi- |
|---|---|
| Amino Acid Group | Conservative Substitutions |
| NonPolar R Groups | Alanine, Valine, Leucine, Isoleucine, |
| | Proline, Methionine, Phenylalanine, |
| | Tryptophan |
| Polar, but uncharged, R Groups | Glycine, Serine, Threonine, Cysteine, |
| | Asparagine, Glutamine |
| Negatively Charged R Groups | Aspartic Acid, Glutamic Acid |
| Positively Charged R Groups | Lysine, Arginine, Histidine |

Another preferred embodiment of the present invention is the use of a purified nucleic acid segment that encodes a protein in accordance with SEQ ID NO:2, SEQ ID NO:4 or SEQ ID NO:20, further defined as a recombinant vector. As used herein, the term “recombinant vector” refers to a vector that has been modified to contain a nucleic acid segment that encodes a HARE protein, or fragment thereof, such as a soluble form of the protein or an HA-binding domain of the protein. The recombinant vector may be further defined as an expression vector comprising a promoter operatively linked to said HARE encoding nucleic acid segment.

Yet another preferred embodiment of the present invention is the use of a purified nucleic acid segment that encodes an active portion of the protein in accordance with a portion of SEQ ID NO:2, SEQ ID NO:4 or SEQ ID NO:20. For example, the invention also includes utilization of a purified nucleic acid segment encoding a soluble form of the protein, such as a portion of the protein containing the extracellular domain but not the cytoplasmic or transmembrane domains of the protein, which retains the ability to bind at least one of HA, chondroitin and chondroitin sulfate, or a portion of the protein containing an active HA-binding domain of HARE.

A further preferred embodiment of the present invention utilizes a host cell, made recombinant with a recombinant vector comprising a HARE gene. In a preferred embodiment, the recombinant host cell is a eukaryotic cell. As used herein, the term “engineered” or “recombinant” cell is intended to refer to a cell into which a recombinant gene, such as a gene encoding HARE, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene, a copy of a genomic gene, or will include genes positioned adjacent to a promoter not naturally associated with the particular introduced gene. In a preferred embodiment, the recombinantly introduced gene may be integrated into the genome of the host cell.

Where one desires to use a eucaryotic host system, such as yeast or Chinese hamster ovary, African green monkey kidney cells, VERO cells, or the like, it will generally be desirable to bring the HARE gene under the control of sequences which are functional in the selected alternative host. In another alternative, the vector may contain a cassette which signals for the sequence to be integrated into the chromosome. The appropriate DNA control sequences, as well as their construction and use, are generally well known in the art as discussed in more detail herein below.

In preferred embodiments, the HARE-encoding DNA segments further include DNA sequences, known in the art functionally as origins of replication or “replicons”, which allow replication of contiguous sequences by the particular host. Such origins allow the preparation of extrachromosomally localized and replicating chimeric segments or plasmids, to which HARE DNA sequences are ligated. In one instance, the employed origin is one capable of replication in bacterial hosts suitable for biotechnology applications. However, for more versatility of cloned DNA segments, it may be desirable to alternatively or even additionally employ origins recognized by other host systems whose use is contemplated (such as in a shuttle vector).

The isolation and use of other replication origins such as the SV40, polyoma or bovine papilloma virus origins, which may be employed for cloning or expression in a number of higher organisms, are well known to those of ordinary skill in the art. In certain embodiments, the invention may thus be defined in terms of a recombinant transformation vector which includes the HARE coding gene sequence together with an appropriate replication origin and under the control of selected control regions.

Thus, it will be appreciated by those of skill in the art that other methods may be used to obtain the HARE gene or cDNA, in light of the present disclosure. For example, polymerase chain reaction or RT-PCR produced DNA fragments may be obtained which contain full complements of genes or cDNAs from a number of sources, including other eukaryotic sources, such as cDNA libraries. Virtually any molecular cloning approach may be employed for the generation of DNA fragments in accordance with the present invention. Thus, the only limitation generally on the particular method employed for DNA isolation is that the isolated nucleic acids should encode a biologically functional equivalent HARE.

Once the DNA has been isolated, it is ligated together with a selected vector. Virtually any cloning vector can be employed to realize advantages in accordance with the invention. Typical useful vectors include plasmids, cosmids, phages and viral vectors for use in prokaryotic or eukaryotic organisms. Examples include pKK223-3, pSA3, pcDNA3.1, recombinant lambda, SV40, polyoma, adenovirus, bovine papilloma virus and retroviruses.

One procedure that would further augment HARE gene copy number is the insertion of multiple copies of the gene into the vector. Another technique would include integrating the HARE gene or multiple copies thereof into chromosomal DNA.

Where a eukaryotic source such as tissues rich in sinusoidal cells of the reticuloendothelial system such as liver, spleen, lymph node and bone marrow is employed, one will desire to proceed initially by preparing a cDNA library. This is carried out first by isolation of mRNA from the above cells, followed by preparation of double stranded cDNA using an enzyme with reverse transcriptase activity and ligation with the selected vector. Numerous possibilities are available and known in the art for the preparation of the double stranded cDNA, and all such techniques are believed to be applicable. A preferred technique involves reverse transcription. Once a population of double stranded cDNAs is obtained, a cDNA library is prepared in the selected host by accepted techniques, such as by ligation into the appropriate vector and amplification in the appropriate host. Due to the high number of clones that are obtained, and the relative ease of screening large numbers of clones by the techniques set forth herein, one may desire to employ phage expression vectors, such as λgt11, λgt12, λGem11, and/or λZAP for the cloning and expression screening of cDNA clones.

In certain other embodiments, the invention concerns isolated DNA segments and recombinant vectors that include within their sequence a nucleic acid sequence essentially as set forth in SEQ ID NO:1, SEQ ID NO:3 or SEQ ID NO:19. The term “essentially as set forth in SEQ ID NO:1”, “essentially as set forth in SEQ ID NO:3” or “essentially as set forth in SEQ ID NO:19” is used in the same sense as described above and means that the nucleic acid sequence substantially corresponds to a portion of SEQ ID NO:1, SEQ ID NO:3 or SEQ ID NO:19, respectively, and has relatively few codons which are not identical, or functionally equivalent, to the codons of SEQ ID NO:1, SEQ ID NO:3 or SEQ ID NO:19, respectively. The term “functionally equivalent codon” is used herein to refer to codons that encode the same amino acid, such as the six codons for arginine or serine, and also refers to codons that encode biologically equivalent amino acids. The term “essentially as set forth in SEQ ID NO:1”, “essentially as set further in SEQ ID NO:3” or “essentially as set forth in SEQ ID NO:19” also incorporates the concept that the encoded protein is functionally equivalent to the protein encoded by SEQ ID NO:1, SEQ ID NO:3 or SEQ ID NO:19, respectively. Thus, pursuant to In Re Wands, Applicants herein disclose conditions and criteria to describe alternate embodiments that could be easily and repeatably determined by one of ordinary skill in the art.

It will also be understood that amino acid and nucleic acid sequences may include additional residues, such as additional N- or C-terminal amino acids or 5′ or 3′ nucleic acid sequences, and yet still be essentially as set forth in one of the sequences disclosed herein, so long as the sequence meets the criteria set forth above, including the maintenance of biological protein activity where protein expression and receptor activity (i.e., HA, chondroitin or chondroitin sulfate binding) is concerned. The addition of terminal sequences particularly applies to nucleic acid sequences which may, for example, include various non-coding sequences flanking either of the 5′ or 3′ portions of the coding region or may include various internal sequences, which are known to occur within genes. The HARE proteins described herein are derived from larger precursor proteins, and therefore such precursor proteins also fall within the scope of the present invention.

Allowing for the degeneracy of the genetic code as well as conserved and semi-conserved substitutions, sequences which have between about 40% and about 80%; or more preferably, between about 80% and about 90%; or even more preferably, between about 90% and about 99%; of nucleotides which are identical to the nucleotides of SEQ ID NO:1, SEQ ID NO:3 or SEQ ID NO:19 will be sequences which are “essentially as set forth in SEQ ID NO:1”, “essentially as set forth in SEQ ID NO:3” or “essentially as set forth in SEQ ID NO:19”, respectively. Sequences which are essentially the same as those set forth in SEQ ID NO:1, SEQ ID NO:3 or SEQ ID NO 19, respectively, may also be functionally defined as sequences which are capable of hybridizing to a nucleic acid segment containing the complement of SEQ ID NO:1 under stringent or relaxed hybridizing conditions. Suitable standard hybridization conditions will be well known to those of skill in the art and are clearly set forth herein.

The term “standard hybridization conditions” as used herein is used to describe those conditions under which substantially complementary nucleic acid segments will form standard Watson-Crick base-pairing. A number of factors are known that determine the specificity of binding or hybridization, such as pH, temperature, salt concentration, the presence of agents, such as formamide and dimethyl sulfoxide, the length of the segments that are hybridizing, and the like. When it is contemplated that shorter nucleic acid segments will be used for hybridization, for example fragments between about 14 and about 100 nucleotides, salt and temperature preferred conditions for hybridization will include 1.2-1.8×HPB (High Phosphate Buffer) at 40-50° C. When it is contemplated that longer nucleic acid segments will be used for hybridization, for example fragments greater than 100 nucleotides, salt and temperature preferred conditions for hybridization will include 1.2-1.8×HPB at 60-70° C.

The term “standard hybridization conditions” includes stringent hybridization conditions as well as relaxed hybridization conditions. In general, when the temperature is increased and salt concentration (ionic strength) is decreased in the wash, the conditions become more stringent; these conditions favor hybrid interactions that have a higher degree of complementarity. When the annealing and wash conditions are at lower temperature and higher ionic strength, less complementary hybrids, which might not be present under more stringent conditions, can be stabilized. For example, to screen the λ-ZAP EXPRESS™ rat LECs cDNA library relatively high-stringency conditions (60° C. overnight in QUIKHYB® hybridization solution (Stratagene, La Jolla, Calif.) followed by two washes for 15 minutes each at room temperature with 2×SSC, 0.1% SDS and two washes for 30 minutes each at 50° C. with 0.1×SSC, 0.1% SDS) were used. However, less stringent hybridization conditions were used to screen a genomic DNA library that was expected to contain numerous exons separated by noncomplementary introns (40° C. overnight in QUIKHYB™ hybridization solution, two washes for 15 minutes each at room temperature with 2×SSC, 0.1% SDS and one wash for 30 minutes at 40° C. with 0.1×SSC-0.1% SDS).

Naturally, the present invention also encompasses DNA segments which are complementary, or essentially complementary, to the sequence set forth in SEQ ID NO:1, SEQ ID NO:3 or SEQ ID NO:19. Nucleic acid sequences which are “complementary” are those which are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment of SEQ ID NO:1, SEQ ID NO:3 or SEQ ID NO:19.

The present invention also includes primers which may be utilized to amplify the coding region of HARE or portions thereof. Nucleic acid segments capable of hybridizing to SEQ ID NO:1, SEQ ID NO:3 or SEQ ID NO:19 in accordance with the present invention are described in copending application U.S. Ser. No. 09/842,930, which has previously been incorporated by reference herein. However, it is to be understood that the present invention is not limited to such primers, and a person of ordinary skill in the art, given this Specification, will be able to identify and select primers which can be utilized to amplify the coding region of HARE, or a portion thereof, such as an extracellular domain or an HA-binding domain of HARE. The present invention also includes primers which are engineered to introduce a restriction site into a DNA sequence to aid in cloning of such DNA sequence. Examples are provided in copending application U.S. Ser. No. 09/842,930 (previously incorporated by reference). However, it is within the skill of one in the art to create restriction sites in a DNA segment which aid in ligation of such DNA segment to a vector having a particular cloning site consisting of a set of restriction sites, and therefore, the present invention is not limited to the primers listed herein.

The nucleic acid segments of the present invention, regardless of the length of the coding sequence itself, may be combined with other DNA sequences, such as promoters, polyadenylation signals, additional restriction enzyme sites, multiple cloning sites, epitope tags, poly histidine regions, other coding segments, and the like, such that their overall length may vary considerably. It is therefore contemplated that a nucleic acid fragment of almost any length may be employed, with the total length preferably being limited by the ease of preparation and use in the intended recombinant DNA protocol.

Naturally, it will also be understood that this invention is not limited to the particular nucleic acid sequences of SEQ ID NO:1, SEQ ID NO:3 and SEQ ID NO:19 and amino acid sequences of SEQ ID NO:2, SEQ ID NO:4 and SEQ ID NO:20. Recombinant vectors and isolated DNA segments may therefore variously include the HARE coding regions themselves, coding regions bearing selected alterations or modifications in the basic coding region, or they may encode larger polypeptides which nevertheless include HARE-coding regions or may encode biologically functional equivalent or precursor proteins or peptides which have variant amino acids sequences.

The DNA segments of the present invention encompass biologically functional equivalent HARE proteins and peptides. Such sequences may arise as a consequence of codon redundancy and functional equivalency which are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques, e.g., to introduce improvements to the functional activity or to antigenicity of the HARE protein.

A preferred embodiment of the present invention utilizes a purified composition comprising a polypeptide having an amino acid sequence in accordance with SEQ ID NO:2 or an amino acid sequence in accordance with SEQ ID NO:4. The term “purified” as used herein, is intended to refer to a HARE protein composition, wherein the HARE protein or appropriately modified HARE protein (e.g. containing a [HIS]₆ tail) is purified to any degree relative to its naturally-obtainable state. The invention also utilizes a purified composition comprising a polypeptide having an amino acid sequence in accordance with a portion of SEQ ID NO:2 or SEQ ID NO:4 wherein the polypeptide is capable of selectively binding at least one of HA, chondroitin and chondroitin sulfate. The ligand blot assay described in detail and utilized in copending application U.S. Ser. No. 09/842,930 (previously incorporated by reference) may be utilized to assay for such an HA-binding domain of HARE.

Turning to the expression of the HARE gene whether from genomic DNA, or a cDNA, one may proceed to prepare an expression system for the recombinant preparation of the HARE protein. The engineering of DNA segment(s) for expression in a eukaryotic system may be performed by techniques generally known to those of skill in recombinant expression.

Another embodiment of the present invention utilizes a method of preparing a protein composition comprising growing a recombinant host cell comprising a vector that encodes a protein which includes an amino acid sequence in accordance with SEQ ID NO:2, SEQ ID NO:4 or SEQ ID NO:20 or an amino acid sequence which is functionally similar with conserved or semi-conserved amino acid changes. The host cell will be grown under conditions permitting nucleic acid expression and protein production followed by recovery of the protein so produced. The production of HARE, including the host cell, conditions permitting nucleic acid expression, protein production and recovery will be known to those of skill in the art in light of the present disclosure of the HARE gene, and the HARE gene protein product HARE, and by the methods described herein.

It is similarly believed that almost any eukaryotic expression system may be utilized for the expression of HARE e.g., baculovirus-based, glutamine synthase-based, dihydrofolate reductase-based systems, SV-40 based, adenovirus-based, cytomegalovirus-based, yeast-based, and the like, could be employed. For expression in this manner, one would position the coding sequences adjacent to and under the control of a promoter. It is understood in the art that to bring a coding sequence under the control of such a promoter, one positions the 5′ end of the transcription initiation site of the transcriptional-reading frame of the protein between about 1 and about 50 nucleotides “downstream” of (i.e., 3′ of) the chosen promoter.

Where eukaryotic expression is contemplated, one will also typically desire to incorporate into the transcriptional unit which includes the HARE gene or DNA, an appropriate polyadenylation site (e.g., 5′-AATAAA-3′) if one was not contained within the original cloned segment. Typically, the poly A addition site is placed about 30 to 2000 nucleotides “downstream” of the termination site of the protein at a position prior to transcription termination.

It is contemplated that virtually any of the commonly employed host cells can be used in connection with the expression of HARE in accordance herewith. Examples of preferred cell lines for expressing HARE cDNA of the present invention include cell lines typically employed for eukaryotic expression such as 239, AtT-20, HepG2, VERO, HeLa, CHO, WI 38, BHK, COS-7, 293, RIN and MDCK cell lines. This will generally include the steps of providing a recombinant host bearing the recombinant DNA segment encoding a functionally active HARE or an active peptide fragment thereof and capable of expressing the functionally active HARE or the active peptide fragment thereof; culturing the recombinant host under conditions that will allow for expression of the recombinant DNA segment; and separating and purifying the functionally active HARE protein or the active peptide fragment thereof which is able to specifically bind at least one of HA, chondroitin and chondroitin sulfate from the recombinant host.

Generally, the conditions appropriate for expression of the cloned HARE gene or cDNA will depend upon the promoter, the vector, and the host system that is employed. For example, where one employs the lac promoter, one will desire to induce transcription through the inclusion of a material that will stimulate lac transcription, such as isopropylthiogalactoside. Where other promoters are employed, different materials may be needed to induce or otherwise up-regulate transcription.

The present invention further utilizes antibodies raised against the Hyaluronan Receptor for Endocytosis (HARE) proteins or fragments thereof described herein, and which are able to selectively bind an epitope of the HARE. In one instance, binding of the antibody to the HARE inhibits the binding of at least one of HA, chondroitin and chondroitin sulfate to HARE and subsequently prevents endocytosis of at least one of HA, chondroitin and chondroitin sulfate by the HARE. Methods of producing such antibodies generally involve immunizing a non-human animal with an immunogenic fragment of the HARE protein. In a preferred embodiment, the immunogenic fragment may comprise an HA-binding domain of HARE. Methods of producing such antibodies are well known to a person of ordinary skill in the art, and therefore no further description is required.

In a preferred embodiment, the antibody utilized in the methods of the present invention is a monoclonal antibody. The term “monoclonal antibody” as used herein refers to a homogenous preparation of antibody molecules, produced by a hybridoma cell line, all of which exhibit the same primary structure and antigenic specificity. That is, all of the antibody molecules of a particular monoclonal antibody preparation recognize and selectively bind the same epitope of HARE. The monoclonal antibodies are produced by methods generally well known to a person of ordinary skill in the art, and briefly involve culturing the hybridoma cell producing the monoclonal antibody specific for HARE under conditions that permit production of such monoclonal antibody.

Such monoclonal antibodies may be utilized to purify functionally active HARE from a biological sample containing HARE via affinity purification. In preferred embodiments, the biological sample may be a tissue rich in sinusoidal cells of the reticuloendothelial system, such as at least one of liver, spleen, lymph nodes and bone marrow. However, it is to be understood that the biological sample may be any sample containing a functionally active HARE.

Affinity purification of proteins utilizing antibodies raised against such proteins is well known to a person of ordinary skill in the art. Briefly, an affinity matrix comprising a monoclonal antibody of the present invention bound to a solid support may be produced by methods well known in the art, and the biological sample may be contacted with the affinity matrix such that HARE in the biological sample binds to the monoclonal antibody of the affinity matrix. The HARE bound to the monoclonal antibody of the affinity matrix may be separated from the remainder of the biological sample by methods well known in the art. The HARE protein is then released from the monoclonal antibody of the affinity matrix and eluted from the affinity column by the addition of a solution, referred to as an eluate, which disrupts the binding between the HARE protein and the antibody. Such eluates are well known in the art, and may include solutions having a lower pH, solutions having a higher salt concentration, and the like. In preferred embodiments, the solution utilized for elution of the HARE protein is based on the ability of the solution to retain the functional activity of the HARE protein. That is, exposure to low pH or high salt may affect the conformations of some proteins, and therefore an eluate is chosen that does not have any effect on the activity of the protein to be eluted.

The monoclonal antibodies of the present invention can also be used to affinity purify peptide fragments of HARE proteins as long as the peptide fragment contains the epitope against which the monoclonal antibody was raised. The monoclonal antibodies of the present invention may also be utilized to affinity purify other proteins (such as the “HARE-like” proteins described herein above) that contain at least one domain or motif similar to a domain or motif of a HARE protein, as long as the corresponding HARE protein domain or motif contains the epitope against which the monoclonal antibody was raised.

In another embodiment of the present invention, a method of identifying compounds which inhibit binding of at least one of HA, chondroitin and chondroitin sulfate to HARE is provided. The method includes providing a purified fragment of HARE capable of binding at least one of HA, chondroitin and chondroitin sulfate and forming a first affinity matrix comprising the purified fragment of HARE bound to a solid support. The first affinity matrix is separated into two portions, and a test compound is contacted with one portion of the first affinity matrix, thereby forming a treated affinity matrix. In two parallel experiments, at least one of HA, chondroitin and chondroitin sulfate that is labeled in such a manner that it can be readily detected is contacted with: (1) the second portion of the first affinity matrix, and (2) the treated affinity matrix. If the HA, chondroitin or chondroitin sulfate binds to a greater extent to the first affinity matrix than to the treated affinity matrix, a determination that the test compound inhibits binding of HA, chondroitin or chondroitin sulfate to HARE can be made. The purified fragment of HARE may be a soluble fragment of HARE, such as an extracellular domain of HARE or an HA-binding domain of HARE.

In yet another embodiment of the present invention, a method of treating a liquid solution containing at least one of HA, chondroitin and chondroitin sulfate is provided. Such method includes providing an affinity matrix comprising a functionally active fragment of HARE, as described herein above, bound to a solid support, and exposing a quantity of the liquid solution to the affinity matrix wherein at least one of HA, chondroitin and chondroitin sulfate contained in the liquid solution is removed therefrom. Such liquid solution could be blood or plasma, such as when blood or plasma is removed from a dialysis patient and filtered to remove contaminants and waste.

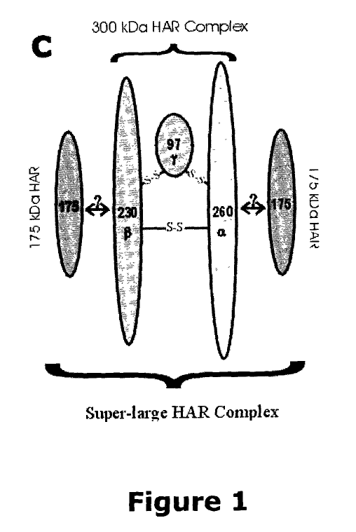

The present invention utilizes the characterization and molecular description of the rat and human HAREs (as described herein below in reference to FIGS. 1-13 and in copending application U.S. Ser. No. 09/842,930) to develop novel strategies to interfere with the metastatic process. In addition, many therapeutic and diagnostic utilities for a functionally active HARE or active peptide fragment thereof, a plasmid encoding same and antibodies which bind thereto are envisioned by the present invention. Such utilities are described in detail herein below. However, various therapies and diagnostic assays utilizing the nucleic acid and amino acid sequences, functionally active peptides and proteins, and antibodies of the present invention can be envisioned, and therefore the present invention is not limited to the methods described herein below.

The monoclonal antibodies (raised against the rat HARE) of the present invention can be utilized in a mammal, such as a human, to target a compound deleterious to tumor cells, such as a radioisotope or chemotherapeutic agent, to such tumor cells when the cancer is present in tissues that express HARE, such as lymph nodes, bone marrow, liver and spleen. When the mammal is a human, the mAb is humanized as described herein and conjugated to the compound/radioisotope/chemotherapeutic agent, and an effective amount of such conjugate is then administered to the individual such that the mAb selectively binds to cells expressing HARE on a surface thereof, thereby delivering the compound/radioisotope/chemotherapeutic agent to the nearby tumor cells which are in close proximity to the cells expressing HARE on the surface thereof.

The mAb/compound conjugate can be targeted to tissues such as lymph node, bone marrow and liver to minimize the chance of metastasis during surgery to remove a primary tumor. The mAb/compound conjugate can also be administered and directed to HARE in such tissues after there is evidence for metastasis.

A similar method can be utilized when it is desired to target a non-deleterious compound to cells expressing HARE on a surface thereof. As in the previous example, the compound is conjugated to a monoclonal antibody of the present invention, and the compound-monoclonal antibody conjugate is administered in an effective amount to a mammal such that the monoclonal antibody selectively binds to cells expressing HARE on a surface thereof, thereby delivering the compound to such cells.

Such utilization of the monoclonal antibodies of the present invention may require administration of such or similar monoclonal antibody to a subject, such as a human. However, when the monoclonal antibodies are produced in a non-human animal, such as a rodent, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the present invention can be “humanized”, that is, the antibodies are engineered such that antigenic portions thereof are removed and like portions of a human antibody are substituted therefor, while the antibodies' affinity for an epitope of HARE is retained. This engineering may only involve a few amino acids, or may include entire framework regions of the antibody, leaving only the complementarity determining regions of the antibody intact. Several methods of humanizing antibodies are known in the art and are disclosed in U.S. Pat. Nos. 6,180,370, issued to Queen et al on Jan. 30, 2001; 6,054,927, issued to Brickell on Apr. 25, 2000; 5,869,619, issued to Studnicka on Feb. 9, 1999; 5,861,155, issued to Lin on Jan. 19, 1999; 5,712,120, issued to Rodriquez et al on Jan. 27, 1998; and 4,816,567, issued to Cabilly et al on Mar. 28, 1989, the Specifications of which are all hereby expressly incorporated herein by reference in their entirety.

In addition, 97 published articles relating to the generation or use of humanized antibodies were identified by a PubMed search of the database. Many of these studies teach useful examples of protocols that can be utilized with the present invention, such as Sandborn et al, Gastroenterology, 120:1330 (2001); Mihara et al, Clin. Immunol. 98:319 (2001); Yenari et al, Neurol. Res. 23:72 (2001); Morales et al, Nucl. Med. Biol. 27:199 (2000); Richards et al, Cancer Res. 59:2096 (1999); Yenari et al, Exp. Neurol. 153:223 (1998); and Shinkura et al, Anticancer Res. 18:1217 (1998), all of which are expressly incorporated in their entirety by reference. For example, a treatment protocol that can be utilized in such a method includes a single dose, generally administered intravenously, of 10-20 mg of humanized mAb per kg (Sandborn, et al. Gastroenterology, 120:1330 (2001)). In some cases, alternative dosing patterns may be appropriate, such as the use of three infusions, administered once every two weeks, of 800 to 1600 μg or even higher amounts of humanized mAb (Richards et al, Cancer Res. 59:2096 (1999)). However, it is to be understood that the invention is not limited to the treatment protocols described above, and other treatment protocols which are known to a person of ordinary skill in the art may be utilized in the methods of the present invention.

The monoclonal antibodies of the present invention may also be utilized in a method of preventing metastasis in an individual wherein the tumor cells of such individual are provided with an HA, chondroitin sulfate or chondroitin coat which interacts with non-tumor cells expressing HARE on a surface thereof. The monoclonal antibody may be humanized as described herein, and an effective amount of the humanized monoclonal antibody can then be administered to the individual such that the humanized monoclonal antibody selectively binds to an epitope of HARE expressed on the surface of the non-tumor cells and inhibits binding of at least one of HA, chondroitin sulfate and chondroitin in the coat of the tumor cells to the non-tumor cells expressing HARE.

An exemplary treatment protocol for use in such a method includes a single dose, generally administered intravenously, of about 10 mg of humanized mAb per kg to about 20 mg of humanized mAb per kg (Sandborn et al. Gastroenterology, 120:1330 (2001)). In some cases, alternative dosing patterns may be appropriate, such as the use of three infusions, administered once every two weeks, of about 800 μg to about 1600 μg or even higher amounts of humanized mAb (Richards et al. Cancer Res. 59:2096 (1999)).

More effective results can be obtained in some patients with a dose in the range of from about 5 mg/kg to about 20 mg/kg taken weekly and administered by subcutaneous injection or by use of an automated delivery device as used for delivery of insulin. However, it is to be understood that the invention is not limited to the treatment protocols described herein above, and other treatment protocols which are known to a person of ordinary skill in the art may be utilized in the methods of the present invention.

While such methods described above involve preventing metastasis by preventing interaction between tumor cells having an HA, chondroitin or chondroitin sulfate coat and non-tumor cells expressing HARE on a surface thereof, the present invention is not limited to such use, and the method described herein above may be utilized to prevent interactions between any cell having an HA, chondroitin or chondroitin sulfate coat and a cell expressing HARE on a surface thereof.

A similar method encompassed by the present invention utilizes a compound other than the humanized monoclonal antibody that inhibits binding of at least one of HA, chondroitin sulfate and chondroitin to HARE, such that upon administration of an effective amount of the compound to the individual described above, the compound inhibits binding of at least one of HA, chondroitin sulfate and chondroitin in the coat of tumor cells to non-tumor cells expressing HARE on a surface thereof. For example, such compound may be any compound that acts as a mimetic for the HA binding site, including a mimetic peptide, a nucleic acid, an oligonucleotide or a PNT (a synthetic DNA formed of protein which mimics oligonucleotides), and conjugates thereof, wherein such compound binds to HARE expressed on the surface of non-tumor cells and inhibits binding of at least one of HA, chondroitin sulfate and chondroitin in the coat of tumor cells to non-tumor cells expressing HARE. However, the invention is not limited to the use of the compounds described herein above as the compound but rather includes any drug or chemical that inhibits HA binding to HARE. Such compounds are identified using an affinity matrix column or multiwell format comprising an HA-, chondroitin sulfate-, or chondroitin-binding domain of HARE bound to a solid support. Upon passing candidate compounds over the immobilized HARE, HA is then passed over the immobilized HARE, and a decrease in HA binding (as detected by methods described herein or known to one of ordinary skill in the art, such as by utilization of HA labeled in such a manner that it can be detected readily) will suggest that such a compound is effective in the method described above.

A treatment protocol for use in such a method includes the same or similar protocol for treatment with a humanized mAb as described previously herein above. Such a treatment protocol would utilize a specific mimetic drug, whether a peptide or other chemical or compound, in the range of from about 5 mg to about 300 mg, and be taken daily and administered by at least one of orally, subcutaneous injection or use of an automated delivery device such as a time release skin patch or a small implanted pump, such as used for delivery of insulin.

While such methods described above involve preventing interaction between tumor cells having an HA, chondroitin or chondroitin sulfate coat and non-tumor cells expressing HARE on a surface thereof, the present invention is not limited to such use, and the method described herein above can be utilized to prevent interactions between any cell having an HA, chondroitin or chondroitin sulfate coat and a cell expressing HARE on a surface thereof.

Another method of the present invention involves targeting a compound to a tissue of a human patient wherein cells of the tissue do not express a functionally active HARE on a surface thereof, but wherein the cells of the tissue express one or more other cell surface or extracellular matrix components capable of binding to HA, chondroitin sulfate or chondroitin, such as but not limited to, CD44. The method involves providing a compound of interest, such as a drug, conjugated to at least one of HA, chondroitin sulfate and chondroitin, which thereby functions as a drug delivery device. By conjugating a drug to HA, chondroitin sulfate or chondroitin and co-administering such conjugate for a therapeutic purpose together with the blocking agents disclosed above to prevent the binding and uptake of HA, chondroitin sulfate or chondroitin to HARE, the lifetime of such drug in the bloodstream or targeted tissues can be prolonged. An effective amount of a humanized monoclonal antibody that selectively binds to an epitope of HARE and inhibits binding of at least one of HA, chondroitin and chondroitin sulfate to HARE, as described in detail herein above, is provided and administered to the human patient such that the humanized monoclonal antibody binds HARE and blocks the binding of at least one of HA, chondroitin sulfate and chondroitin to HARE, so that upon administration of an effective amount of the compound-HA, compound-chondroitin sulfate or compound-chondroitin conjugate to the human patient, the compound-HA, compound-chondroitin sulfate or compound-chondroitin conjugate is not able to bind to the cells expressing HARE and is therefore delivered to the cells of a tissue which do not express HARE on a surface thereof.

A treatment protocol for use in such a method includes the same or similar protocol for treatment with a humanized mAb as described herein above. Such a treatment protocol would utilize a specific mimetic drug, whether a peptide or other chemical or compound, could be in the range of from about 5 mg to about 300 mg taken daily and administered orally, by subcutaneous injection or by use of an automated delivery device such as a time release skin patch or a small implanted pump, such as used for delivery of insulin.

In a similar manner, if one desires to target a compound of interest, such as a drug, to a tissue of an individual wherein cells of the tissue express HARE on a surface thereof, the method above may be utilized with the exception that the humanized monoclonal antibody is omitted. That is, the method includes conjugating the compound to an HA, chondroitin sulfate or chondroitin molecule or a desired combination thereof (which acts as a drug delivery device, as described herein before), and administering an effective amount of the HA-, chondroitin sulfate- and/or chondroitin-compound conjugate to the individual such that the HARE expressed on the surface of cells in the tissue bind and endocytose the HA-, chondroitin sulfate- and/or chondroitin-compound complex, thereby delivering the HA-, chondroitin sulfate- and/or chondroitin-compound complex to the cells of such tissue.

The compound-HA, compound-chondroitin or compound-chondroitin sulfate conjugate can be targeted to tissues such as lymph node, bone marrow and liver to minimize the chance of metastasis during surgery to remove a primary tumor. The compound-HA, compound-chondroitin or compound-chondroitin sulfate conjugate can also be administered and directed to HARE in such tissues after there is evidence for metastasis.

A treatment protocol that could be utilized in such a method includes a specific drug, whether a peptide or other chemical or compound, conjugated to HA, chondroitin sulfate and/or chondroitin and used at a dose in the range of from about 5 mg to about 300 mg taken daily and administered either by intravenous injection, by subcutaneous injection or by use of an automated delivery device such as a time release skin patch or a small implanted pump, such as used for delivery of insulin.

Other methods envisioned by the present invention involve methods of treating a disease in a patient wherein one symptom of the disease is an elevated level of at least one of HA, chondroitin and chondroitin sulfate in the blood or lymph. In one embodiment, the method comprises administering to a patient an effective amount of a plasmid, cosmid, phage, viral vector or other vector encoding a functionally active HARE. The vector should be targeted to a specific cell type such that upon transfection or transduction of such cell with such vector, the cell expresses increased levels of HARE on the surface thereof. This allows such cell to endocytose greater amounts of HA, chondroitin and chondroitin sulfate and thereby clear an increased amount of HA, chondroitin or chondroitin sulfate from the circulation. Preferably, the vector is targeted to a cell that normally expresses HARE and endocytoses HA, chondroitin or chondroitin sulfate, such as but not limited to, reticuloendothelial cells of the liver and the lymphatic system.

In another embodiment, an affinity matrix is formed which comprises a functionally active fragment of HARE bound to a solid support. Through the process of dialysis, the patient's blood or plasma may be exposed to the affinity matrix such that excess HA, chondroitin or chondroitin sulfate in the patient's blood or plasma binds to the functionally active fragment of HARE of the affinity matrix and is thereby removed from the patient's blood or plasma.

In yet another embodiment, an “artificial organ” is created by expressing the HARE gene in compatible cells, which could preferably be the patient's own cells, and using these cells either in culture in vitro or reinfused back into the patient in vivo to clear HA, chondroitin and/or chondroitin sulfate from blood or plasma.

A treatment protocol that could be utilized in such a method includes the isolation under sterile conditions of the patient's white blood cells and their exposure, by transfection, transduction or other appropriate method, to a plasmid, cosmid, phage, viral vector or other vector encoding a functionally active HARE such that the recipient cells then express an active HARE capable of binding and internalizing HA, chondroitin sulfate and/or chondroitin from the surrounding milieu. The patient's cells are then transfused back into the patient wherein these cells containing HARE are then able to lower the blood concentration of HA, chondroitin sulfate and/or chondroitin as desired.

In a further embodiment of the present invention, a soluble fragment of HARE that retains the ability to specifically bind at least one of HA, chondroitin and chondroitin sulfate is utilized to detect HA, chondroitin or chondroitin sulfate in a variety of applications, including ELISA assays and immunocytochemistry. Such soluble fragment of HARE may be an extracellular domain of HARE or an HA-binding domain, a chondroitin-binding domain or a chondroitin-sulfate binding domain of HARE. Clinically, the soluble fragment of HARE could be used to make a test kit for measurement of urine or serum levels of HA, chondroitin and/or chondroitin sulfate, such information as may be needed for diagnostic procedures, particularly those related to diseases and cancers that are accompanied by significant elevations of the circulating levels of HA.

A protocol that could be utilized in such a method includes immobilizing the HARE-derived protein domain on a solid support by methods known to those in the art, such as by covalent attachment of the HARE-derived protein domain to a bead support, such as CNBr-activated Sepharose, and establishment of a negative competition binding assay in which a radiolabeled, biotinylated, fluorescently labeled or otherwise suitably tagged preparation of HA is allowed to bind to the solid HARE-containing support in the absence and presence of increasing amounts of the liquid sample to be tested. Based on a standard curve with known amounts of nonlabeled HA, the amount of HA, chondroitin sulfate or chondroitin present in the sample can be calculated. If desired, identification of the particular glycosaminoglycan present among HA, chondroitin sulfate or chondroitin can be further elucidated by utilizing treatment of the sample with specific glycosidases to differentiate the various contributions to the overall assay result by each of either HA, chondroitin sulfate or chondroitin, and the amount of HA, chondroitin and/or chondroitin sulfate in the sample can be quantitated.

In a similar manner as described above for the negative competition binding assay, one can also develop a capture assay for measuring levels of HA, chondroitin or chondroitin sulfate in a sample, such as a biological fluid. A HARE fragment, such as the HA, chondroitin and/or chondroitin sulfate binding regions of HARE, is immobilized by attachment to a solid phase. A sample is contacted with the immobilized fragment, thereby allowing HA, chondroitin or chondroitin sulfate present in the sample to bind to the immobilized HARE protein or peptide fragment. The sample is then washed away, and a labeled HARE protein (or labeled HARE peptide containing the HA, chondroitin and/or chondroitin sulfate binding domains) is used to detect HA, chondroitin or chondroitin sulfate bound to the immobilized HARE protein or peptide fragment.

It is to be understood that test kits for measurements of HA, chondroitin and/or chondroitin sulfate in a sample utilizing the negative competition assay or the capture assay both fall within the scope of the present invention. A test kit which could be utilized for detecting HA, chondroitin and/or chondroitin sulfate by the negative competition assay comprises an immobilized HARE protein or an immobilized HARE peptide fragment that contains the HA, chondroitin and/or chondroitin sulfate binding domains, a labeled or tagged preparation of HA, means for contacting the sample with a portion of the immobilized HARE protein or peptide fragment to form a mixture thereof, and means for contacting the labeled or tagged preparation of HA with immobilized HARE protein or peptide fragment alone and with the mixture of sample and immobilized HARE protein or peptide fragment. The kit may further include a known amount of nonlabeled HA for preparing a standard curve for calculating the amount of HA, chondroitin or chondroitin sulfate present in the sample. In addition, the kit may also further include at least one specific glycosidase for identifying the particular glycosaminoglycans present among HA, chondroitin and chondroitin sulfate in the sample.

A test kit which could be utilized for detecting HA, chondroitin and/or chondroitin sulfate by the capture assay comprises an immobilized HARE protein or an immobilized HARE peptide fragment that contains the HA, chondroitin and/or chondroitin sulfate binding domains, a labeled or tagged preparation of HARE protein or HARE peptide fragment that contains the HA, chondroitin and/or chondroitin sulfate binding domains, means for contacting the sample with a portion of the immobilized HARE protein or peptide fragment to form a mixture thereof, means for washing away unbound sample, and means for contacting the labeled or tagged preparation of HARE protein or peptide fragment with HA, chondroitin and/or chondroitin sulfate (present in the sample) bound to the immobilized HARE protein or peptide fragment. In addition, the kit may further include at least one specific glycosidase for identifying the particular glycosaminoglycans present among HA, chondroitin and chondroitin sulfate in the sample.

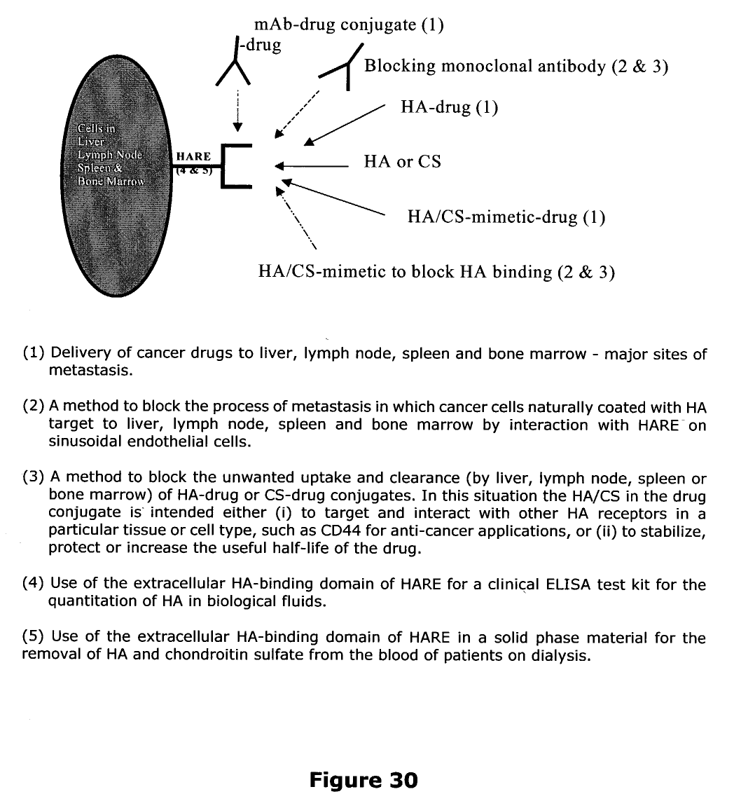

FIG. 30 provides a schematic illustration of some of the above-described methods of the present invention.

The following examples illustrate the practice of the preferred embodiments of the present invention. However, the present invention is not limited to the examples set forth.

### EXAMPLE

U.S. Ser. No. 09/842,930, which has previously been incorporated herein by reference, discloses the identification and characterization of functionally active Hyaluronan Receptor for Endocytosis (HARE) from rat and human liver which are both able to specifically bind at least one of HA, chondroitin and chondroitin sulfate and endocytose the bound HA, chondroitin or chondroitin sulfate into a cell via a clathrin-coated pit pathway. U.S. Ser. No. 09/842,930 also discloses the isolation of monoclonal antibodies raised against an HA-binding domain of rat HARE, wherein at least one of the monoclonal antibodies blocks binding of HA to HARE. FIGS. 1-13 are provided herein to summarize the identification and characterization of the rat and liver HAREs as well as the isolation of such monoclonal antibodies against the HA-binding domain of rat HARE.

### Description of FIGS. 1-13

U.S. Ser. No. 09/842,930 describes the isolation and characterization of two rat liver HARE isoreceptors that are present in liver, spleen and lymph node. The 175 kDa and 300 kDa HARE species are independent isoreceptors, and the 175 kDa HARE is a bonefide endocytic receptor for HA that is capable of functioning independently of the 300 kDa HARE. Although it is possible that the 175 kDa HARE and 300 kDa HARE species could function together as a large complex (as illustrated in FIG. 1), it is apparently not necessary for these two HAREs to be present in the same cell in order to create a specific functional HA receptor. The two HARE isoreceptors may be necessary to mediate HA uptake and degradation in mammals because of the extremely broad range of HA molecular masses present in tissues throughout the body. The two isoreceptors could have different preferences for the size of the HA with which they interact. Presumably, the smaller HARE would interact with smaller HA and the larger HARE with larger HA.

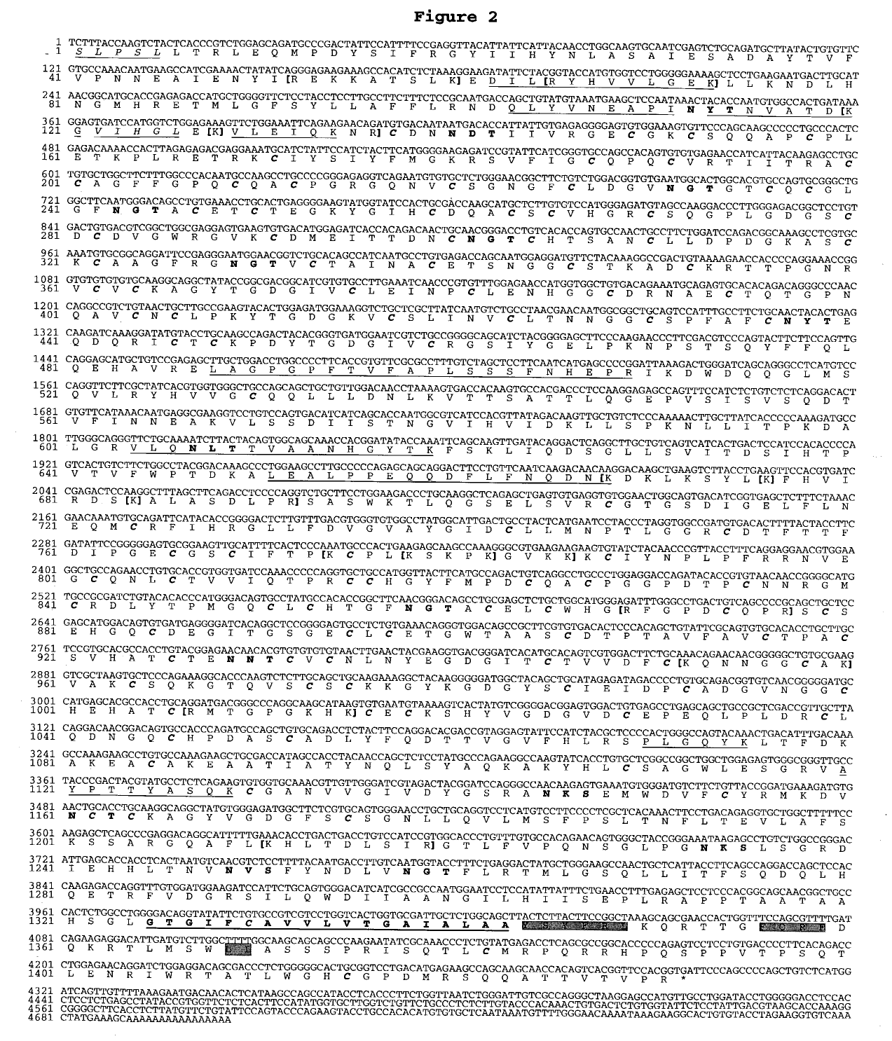

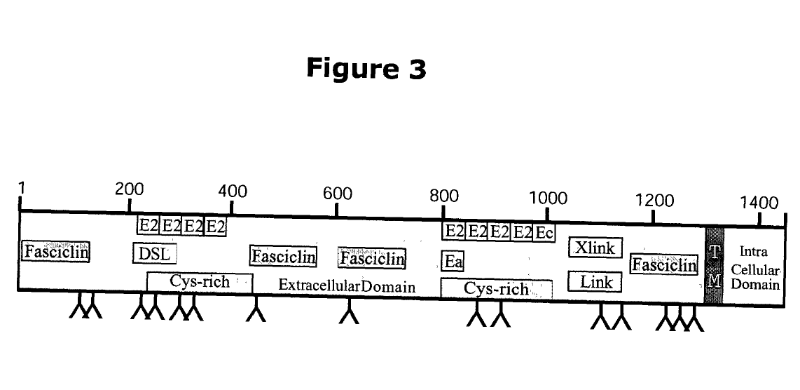

FIG. 2 illustrates the cDNA sequence (SEQ ID NO:1) of the deduced 175 kDa HARE, which encodes a 1431 amino acid protein (SEQ ID NO:2). The protein is predicted to be a type I membrane protein (FIG. 3), with a large NH₂-terminal extracellular domain (1322-1324 residues depending on the particular prediction program used), a single transmembrane domain (˜L¹³²³- A¹³⁴³), and a small COOH-terminal cytoplasmic domain (˜88 amino acids). As is the case for many proteins, the exact boundaries predicted for the transmembrane domain of HARE are somewhat uncertain; they vary by 2-3 amino acids on both sides of the predicted domain depending on the particular algorithm used. For example, the programs TMPred, TMHMM and PSORTII, respectively, predict a transmembrane domain between residues 1327-1347, 1325-1347 and 1327-1343. The predicted mass of the protein is 156,002 Da, and the predicted isoelectric point is pH 7.49. The ectodomain contains 15 putative N-glycosylation sites (excluding one NPS sequon), and two cysteine-rich regions. The extracellular domain has multiple motifs and subdomains with homology to similar regions identified in other receptors and matrix molecules. Multiple EGF-like, βIgH3, and Fasciclin domains, as well as one DSL domain, are also organized throughout the extracellular domain of the 175 kDa HARE. In addition, a 93 amino acid region near the membrane junction (Gly¹⁰⁶³- Arg¹¹⁵⁶) is homologous to the mammalian proteoglycan extracellular Xlink domain and the HA-binding domain of the link protein.

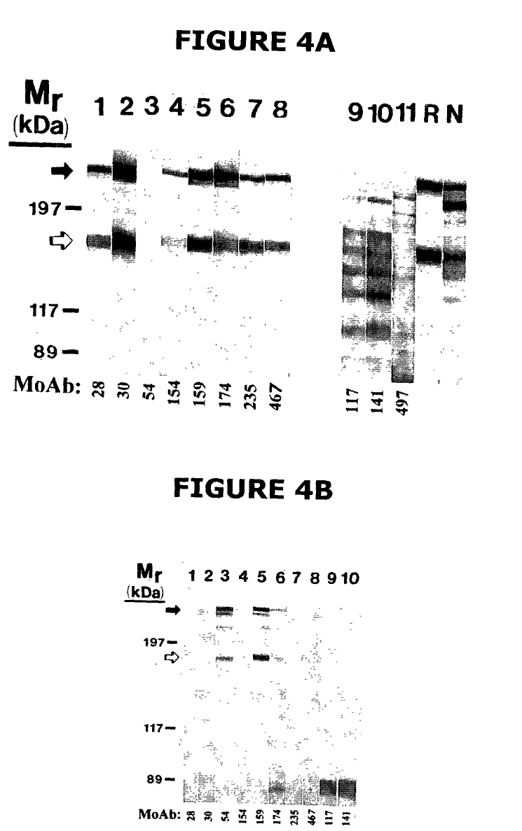

Antibodies were raised utilizing a partially purified fragment of the 175 kDa rat HARE as the antigen, and eleven original monoclonal antibodies were selected as candidates. Eight of the 11 mAbs recognize both the rat LEC 175 HARE and 300 HARE in Western blots after either nonreducing (FIG. 4A) or reducing (FIG. 4B) SDS-PAGE (mAb's 117, 141 and 497 were not against 175 HARE, since they have a different Western pattern and do not immunoprecipitate HARE). Three mAbs (numbers 54, 159 and 174) recognize both reduced HAREs in Western blots. Most of the mAbs raised against the nonreduced 175 HARE no longer react with either HARE species after reduction (FIGS. 4A and 4B). The exceptions are mAb-159 and mAb-174, which recognize both the 175 HARE and 300 HARE proteins in Western blots, whether they are reduced (FIG. 4B) or nonreduced (FIG. 4B). MAb-54 recognizes only the reduced HAREs (FIGS. 4A and 4B, lanes 3).

Four of the mAbs also immunoprecipitate both proteins from LEC extracts. Surprisingly, all mAbs that bind to the 175 HARE species, the original antigen, also recognize the 300 HARE species. However, as described below, the 300 kDa species is not a dimer of the 175 kDa protein and does not contain a 175 kDa subunit. That eight of eight mAbs raised against the 175 HARE cross-react with the 300 HARE suggests that the two proteins share one or more common epitopes that may be very antigenic. Except for mAb-159 (IgM) and mAb-30 (IgG_{2b}), all of the HARE-specific mAbs are IgG₁. Listed in Table II are the characteristics of the eight mAbs raised against the rat 175 HARE.

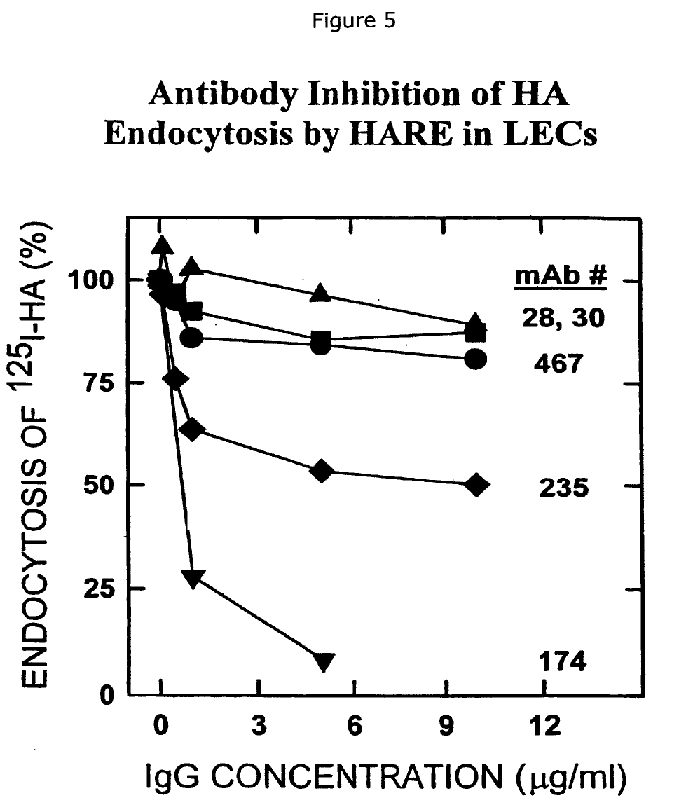

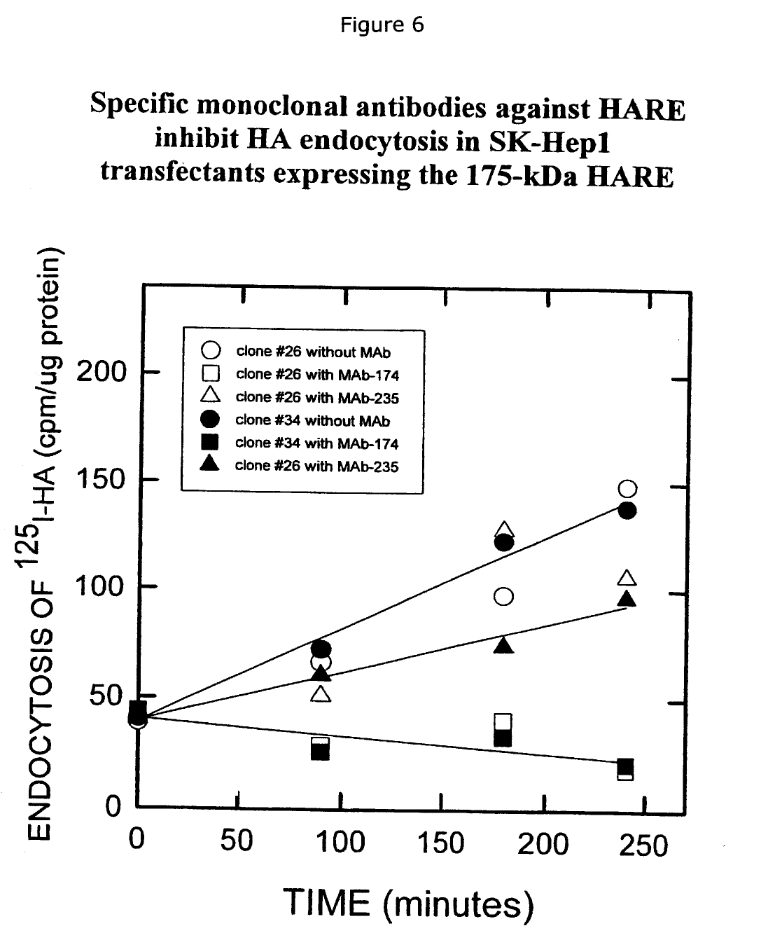

FIGS. 5 and 6 illustrate the specificity of monoclonal antibodies raised against the rat liver 175 kDa HARE protein. Endocytosis and accumulation of ¹²⁵I-HA at 37° C. by cultured LECs was completely inhibited by MAb-174 (FIG. 5). Only one other MAb (#235) had any appreciable affect on HA endocytosis, consistently causing partial (about 50%) inhibition of ¹²⁵I-HA endocytosis. The same results were seen with a SK-Hep1 cell line transfected with cDNA encoding a recombinant 175-kDa HARE (FIG. 6).

Western blot analysis and confocal indirect immunofluorescence demonstrated that the HARE proteins are expressed in spleen as well as in liver, but are not present or are present at much lower levels in brain, lung, heart, muscle, kidney and intestine. The HARE proteins are localized to the sinusoids in the liver and were not observed in parenchymal cells. In addition, the protein is not expressed in isolated hepatocytes in culture but is strongly expressed in purified, cultured LECs, in a pattern typical for an endocytic, recycling receptor: at the cell surface, in pericellular vesicles (presumably endosomes), ER and Golgi. In rat spleen, the HARE proteins are present in the venous sinuses of the red pulp, and were not observed in the germinal centers or white pulp of the splenic nodules. In rat lymph nodes, HARE is localized to the medullary sinuses and is not present in the spheroid nodules or their germinal centers.

The domain organization of HARE is very different from that of all the other known HA-binding proteins or HA receptors including ICAM-1, RHAMM (also recently designated CD168), CD44, TSG-6, Link protein and LYVE-1. We and others have noted the presence, in various genomic and EST databases, of protein sequences with significant homology to several known HA-binding proteins. For example, a group of three ORFs were reported to encode HA-binding

TABLE II
Characteristics of mAbs against the rat and human HARE
isoreceptors The 8 mAbs raised against the rat liver 175
kDa HARE were tested for their usefulness (+, yes; −, no)
as reagents: for immunoprecipitation or Western blot (WB)
analysis of either the rat or human small (175-190 kDa)
or large (300-315 kDa) HARE proteins; for inhibition of
HA binding to LECs or to either HARE in a ligand
blot assay; and for immunocytochemical analysis of
HARE expression in rat or human tissues.

| | Mouse Monoclonal Antibody Number | | | | | | | |
|---|---|---|---|---|---|---|---|---|
| Property | 28 | 30 | 54 | 154 | 159 | 174 | 235 | 467 |
| Immunoprecipitation | + | + | − | − | + | + | + | + |
| of the rat | | | | | | | | |
| 175 kDa HARE | | | | | | | | |
| Immunoprecipitation | + | + | − | − | + | + | + | + |
| of the rat | | | | | | | | |
| 300 kDa HARE | | | | | | | | |
| Recognizes | + | + | − | + | + | + | + | + |
| nonreduced rat | | | | | | | | |
| 175 kDa HARE in WB | | | | | | | | |
| Recognizes | + | + | − | + | + | + | + | + |
| nonreduced rat | | | | | | | | |
| 300 kDa HARE in WB | | | | | | | | |
| Recognizes reduced | − | − | + | − | + | ˜ + | − | − |
| rat 175 kDa | | | | | | | | |
| HARE in WB | | | | | | | | |
| Recognizes 260 kDa | − | − | + | − | + | ˜ + | − | − |
| subunit of rat | | | | | | | | |
| 300 kDa HARE in WB | | | | | | | | |
| Recognizes 230 kDa | − | − | + | − | + | ˜ + | − | − |
| subunit of rat | | | | | | | | |
| 300 kDa HARE in WB | | | | | | | | |
| Recognizes 97 kDa | − | − | − | − | − | − | − | − |
| subunit of rat | | | | | | | | |
| 300 kDa HARE in WB | | | | | | | | |
| Blocks HA uptake in | − | − | − | − | − | + | + | − |
| rat LECs at | | | | | | | | |
| 37-degrees | | | | | | | | |
| Blocks HA binding | − | − | − | − | − | + | − | − |
| to 175 kDa | | | | | | | | |
| HARE in blots | | | | | | | | |
| Blocks HA binding | − | − | − | − | − | + | − | − |
| to 300 kDa | | | | | | | | |
| HARE in blots | | | | | | | | |
| Immunocytochemistry | + | + | + | + | + | + | + | + |
| of rat tissues | | | | | | | | |
| Immunoprecipitation | − | + | − | − | − | − | − | − |
| of the human | | | | | | | | |
| 190 kDa HARE | | | | | | | | |
| Immunoprecipitation | − | + | − | − | − | − | − | − |
| of the human | | | | | | | | |
| 315 kDa HARE | | | | | | | | |
| Recognizes | − | + | − | + | − | − | − | − |
| nonreduced human | | | | | | | | |
| 190 kDa HARE in WB | | | | | | | | |
| Recognizes | − | + | − | + | − | − | − | − |
| nonreduced human | | | | | | | | |
| 315 kDa HARE in WB | | | | | | | | |
| Recognizes reduced | − | − | − | − | + | − | − | − |
| human 190 | | | | | | | | |
| kDa HARE in WB | | | | | | | | |
| Recognizes 250 kDa | − | − | − | − | + | − | − | − |
| subunit of human | | | | | | | | |
| 315 kDa HARE in WB | | | | | | | | |
| Recognizes 220 kDa | − | − | − | − | + | − | − | − |
| subunit of human | | | | | | | | |
| 315 kDa HARE in WB | | | | | | | | |
| Immunocytochemistry | − | + | − | + | + | − | − | − |
| of human tissues | | | | | | | | |

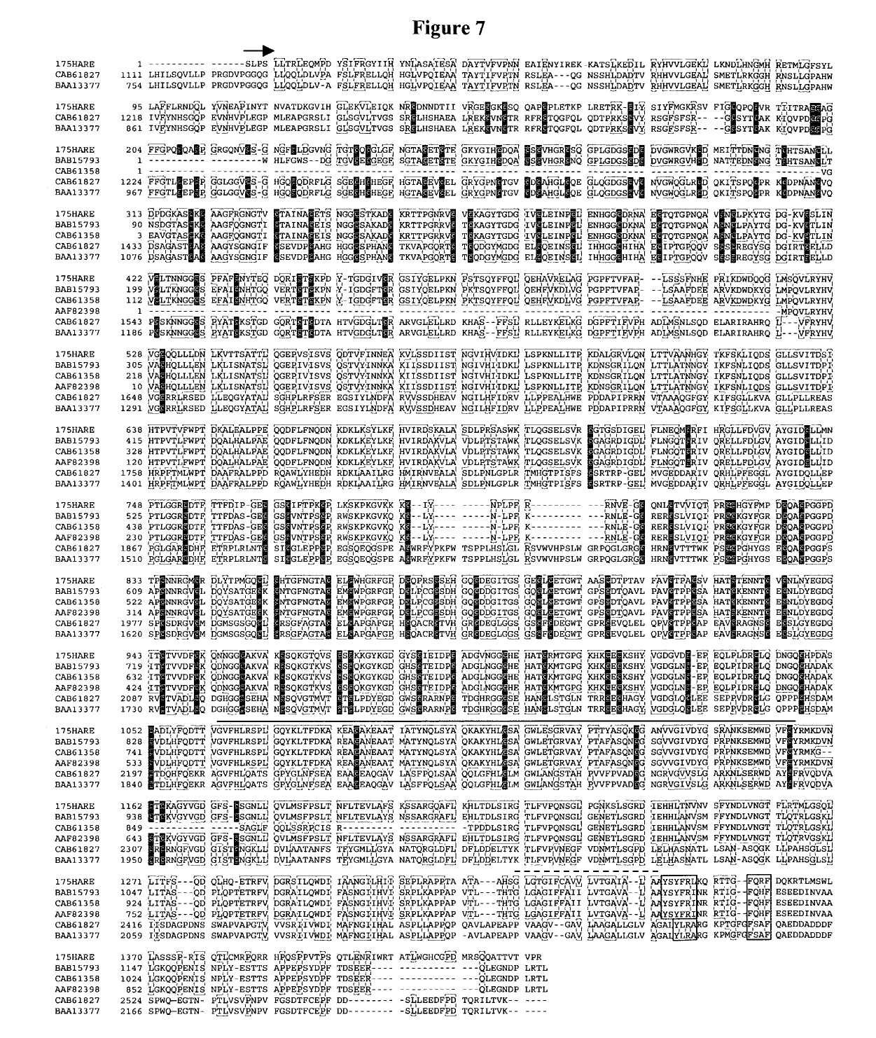

proteins based on the fact that the deduced protein sequences contained a Link-like domain with homology to the Link protein (Tsifrina et al, Am. J. Pathol. 155:1625 (1999)). HARE is highly related to these putative HA binding proteins (FIG. 7), which constitute a family of membrane-bound HA receptors, with the 175 kDa HARE as the prototype and first functionally identified member.

Three of the monoclonal antibodies raised against the rat 175 kDa HARE (numbers 30, 154 and 159) were able to recognize a human HARE homologue in human spleen. As observed with the rat HARE, two high molecular weight protein species, at ˜190 kDa and ˜315 kDa, were reactive with the mAbs are were able to bind HA. The specific reactivity of the human HARE proteins with mAb-30, which had been used to purify the rat liver HARE, enabled the purification of the HARE proteins directly from detergent extracts of human spleen by immunoaffinity chromatography. The ˜315 kDa HARE is consistently more abundant than the 190 kDa HARE in human spleen. The apparent molar ratio of the ˜315 kDa HARE: 190 kDa HARE in spleen is ˜2-3:1. Interestingly, essentially the reverse ratio is observed for the two HARE isoreceptors in rat liver.

Upon subunit characterization of the two human HARE isoreceptors, it was determined that the 190 kDa HARE contains only one polypeptide, which migrates at ˜196 kDa after reduction. The ˜315 kDa HARE contains at least two types of disulfide-bonded subunits, which migrate at ˜220 kDa and ˜250 kDa upon reduction. The apparent molar ratio of 250 kDa: 220 kDa subunits is about 2-3:1. In contrast, the rat 300 kDa HARE contains three subunits of 97, 230 and 260 kDa in apparent molar ratios of 1:1:1, respectively.

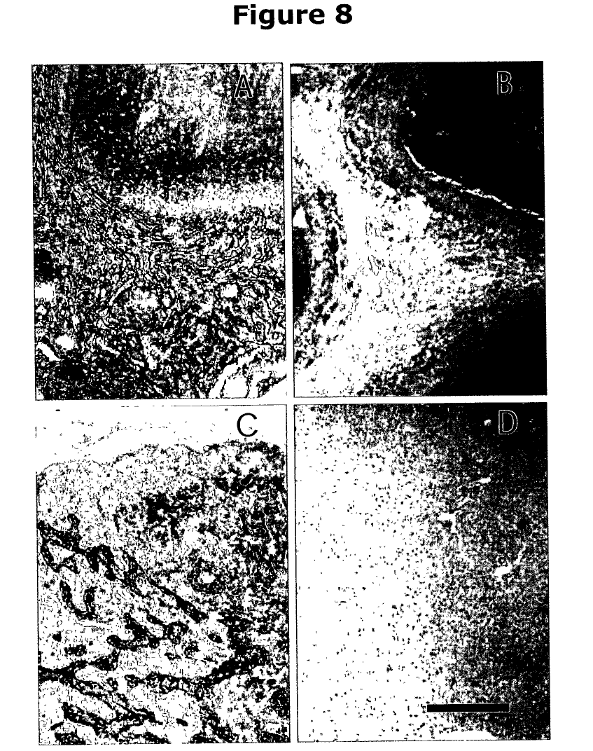

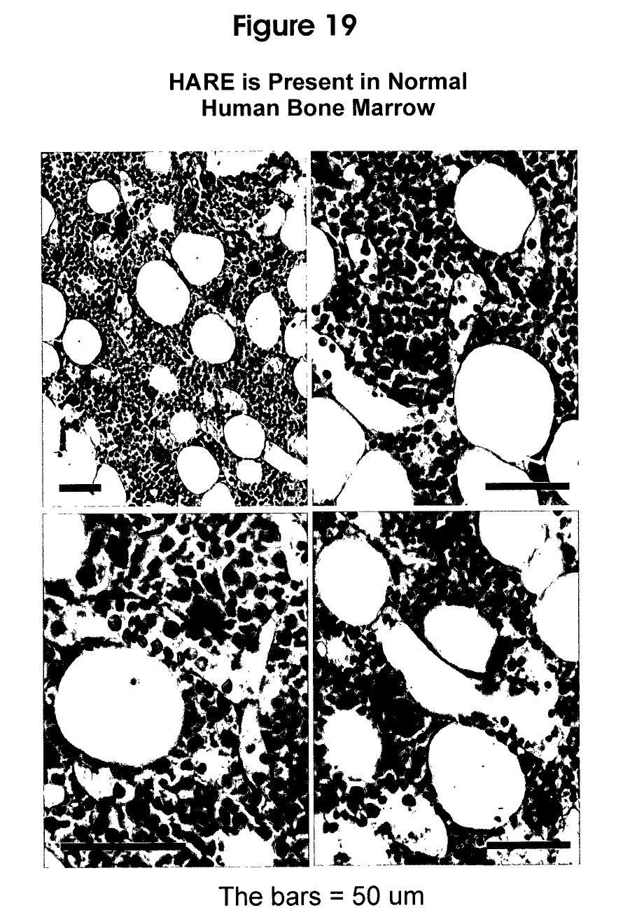

Using mAb-30, abundant HARE protein expression was found in human liver, spleen and lymph node (FIG. 8) and in bone marrow (FIG. 19). Staining intensity, and therefore protein expression levels, were much greater in lymph node than in spleen than in liver. In each tissue, only cells in the sinusoidal regions were stained. In spleen, the germinal centers and white pulp areas of spleenic nodules were unstained, whereas the venous sinusoids of the red pulp stained strongly. A more thorough examination of other human tissues is still in progress.

When the protein databases were searched using amino acid sequences derived from the affinity purified HARE proteins, an identical match was found with two different subsets of peptides predicted to be within a hypothetical human protein of unknown function under accession number BAB15793. This sequence had also been independently identified (FIG. 7) as the most likely human homologue of HARE based on overall homology of ˜85% (78% identity) between the 1431 amino acid rat 175 kDa HARE and a putative 1193 amino acid protein encoded by BAB15793. RT-PCR with human spleen mRNA and a combination of human HARE-specific and BAB15793-specific primers was utilized to identify, clone and sequence PCR products that span portions of the HARE-coding sequence, therefore further supporting the relationship between the purified human spleen HARE and the partial protein sequence deduced from BAB15793.

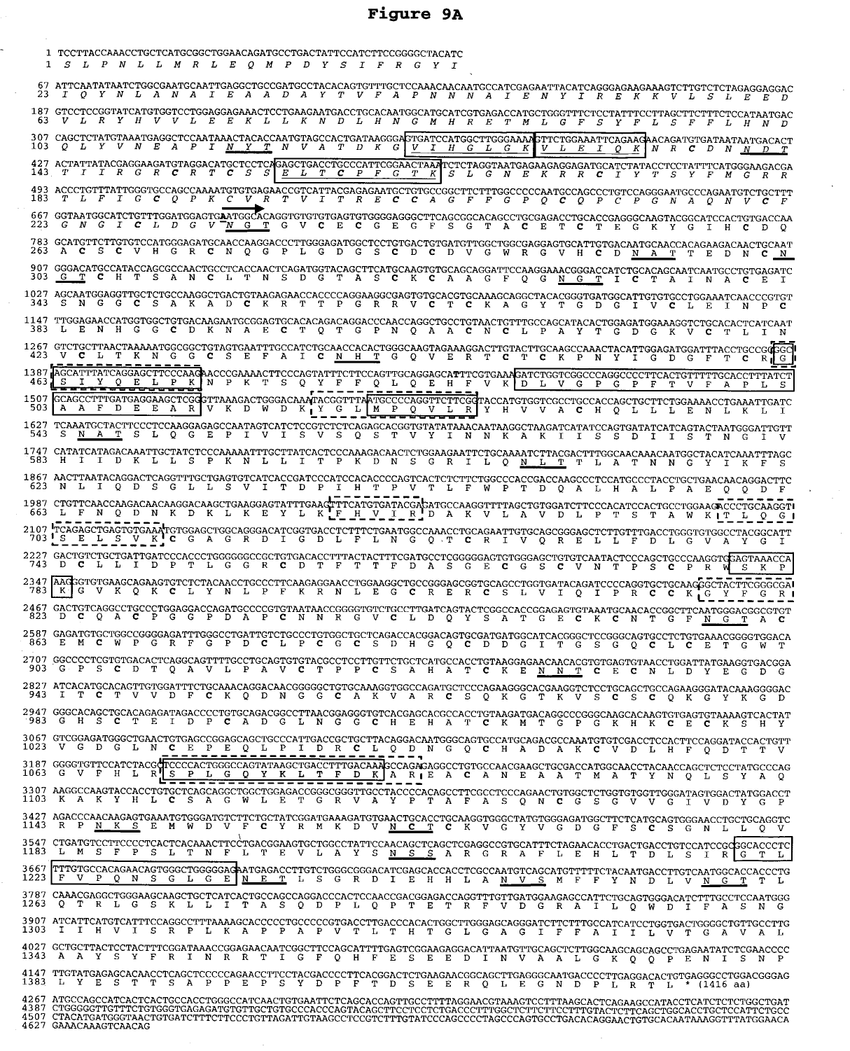

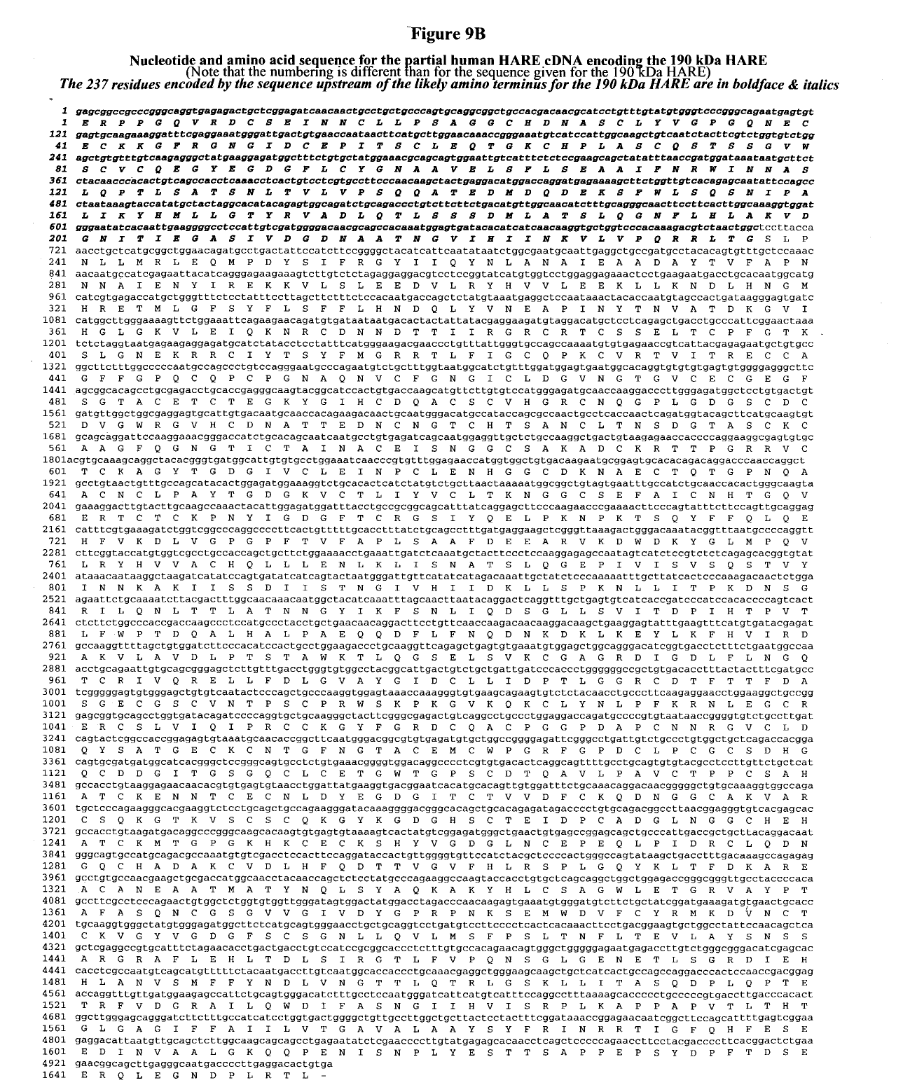

The nucleic acid sequence (SEQ ID NO:3) and deduced protein sequence (SEQ ID NO:4) for the 190 kDa human HARE are shown in FIG. 9A. The BAB15793 nucleotide sequence contains a partial ORF of 1193 amino acids that starts at nucleotide position 606. The RT-PCR products generated from spleen mRNA confirmed almost all of the 4575 bp BAB15793 sequence with several important exceptions. Most significantly, key results characterizing new human HARE sequences were obtained from the most 5′ PCR product that was derived from an upstream region of BAB15793 that had been incorrectly concluded to be untranslated. The majority of this 418 bp PCR product is upstream of the putative Trp residue (see FIG. 7) that begins the BAB15793 hypothetical protein sequence (FIG. 9). In fact, the first seven residues of this hypothetical sequence were incorrect due to a frameshift error. Other PCR products are in-frame with, and extend the size of, the human HARE ORF to at least 4251 bp, ending at a stop codon and encoding a protein of 1416 residues. This additional deduced protein sequence contains another three tryptic peptides identified from the purified HARE protein and is 83% identical to the same 139 residue region in the rat 175 kDa HARE.

The entire 1416 amino acid open reading frame (4251 nucleotides) of the human 190 kDa HARE (SEQ ID NO:4) has been successfully amplified from a human lymph node cDNA library. A similar bp PCR product was also seen with a comparable cDNA library prepared from human spleen.

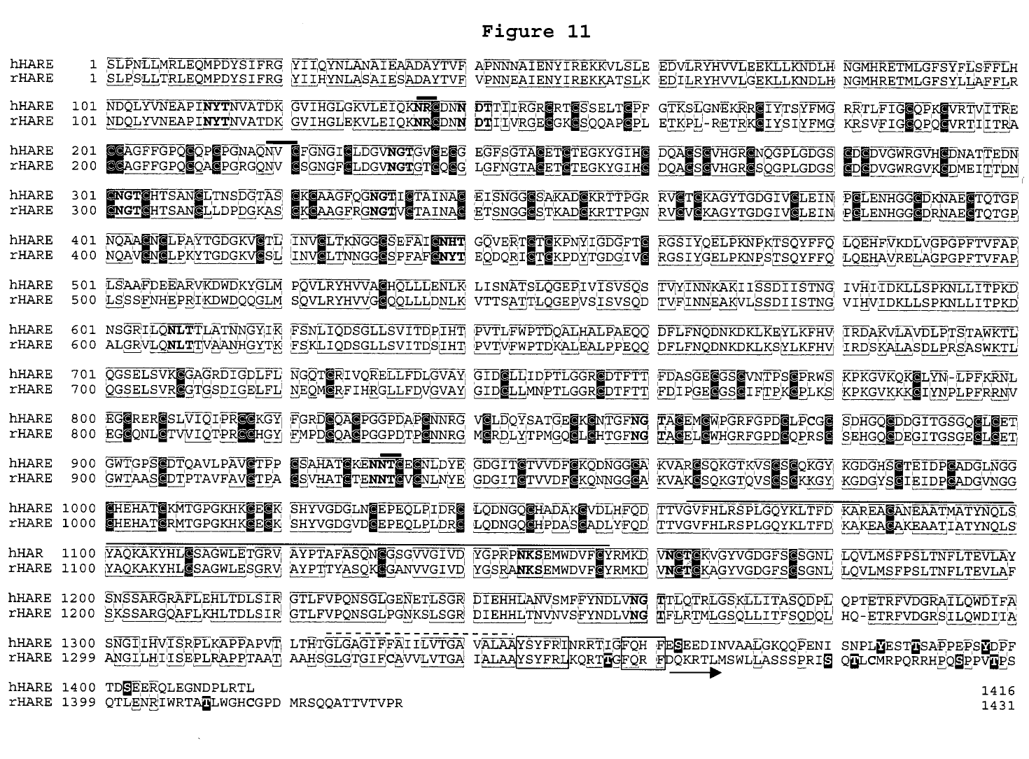

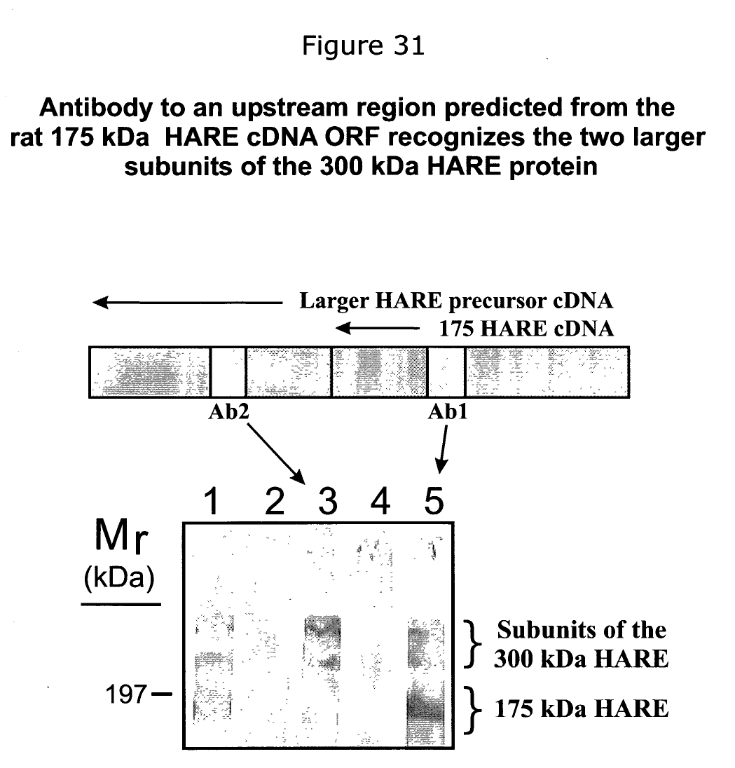

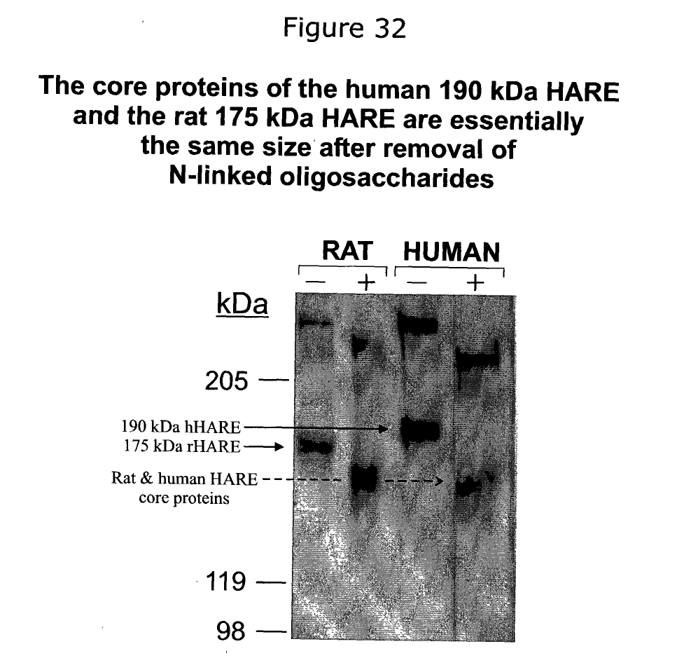

The human partial cDNA encoding the 190 kDa HARE in fact encodes for a much larger protein which is consistent with the finding for the rat HARE that a large precursor protein gives rise to the smaller HARE. For example, FIG. 31 demonstrates that the two largest rat HARE proteins were reactive with an antibody against a predicted amino acid sequence upstream of the cDNA region encoding the native rat 175-kDa HARE. Furthermore, the partial human cDNA for HARE encodes a protein with almost the identical N-terminal 20-residue sequence found for the rat 175 kDa HARE (FIG. 11). This human core protein for the 190 kDa HARE corresponds with a very high level of identity and similarity to the rat 175 kDa HARE protein. Despite the apparent size difference between the human 190 kDa and rat 175 kDa HARE species, the sizes of the two core proteins are identical, as evidenced in FIG. 32. In this experiment, the affinity purified proteins were treated with endoglycosidase F to remove N-linked oligosaccharides and then analyzed by SDS-PAGE and Western blotting to detect the human and rat HARE core proteins.

Based on all of these above results, it is evident that the human cDNA sequence encoding the 190 kDa HARE has been identified and assembled. Since a human cDNA library from which the complete 4251 bp PCR product can be amplified has been identified, the appropriate complete cDNAs for the 300 kDa HARE protein, which is the precursor for the smaller HARE, can also be cloned. Therefore, the present invention is not limited to the cDNAs disclosed herein, but further encompasses the complete cDNA for human HARE which can be obtained using standard procedures (including the human genome databases) known to a person of ordinary skill in the art.

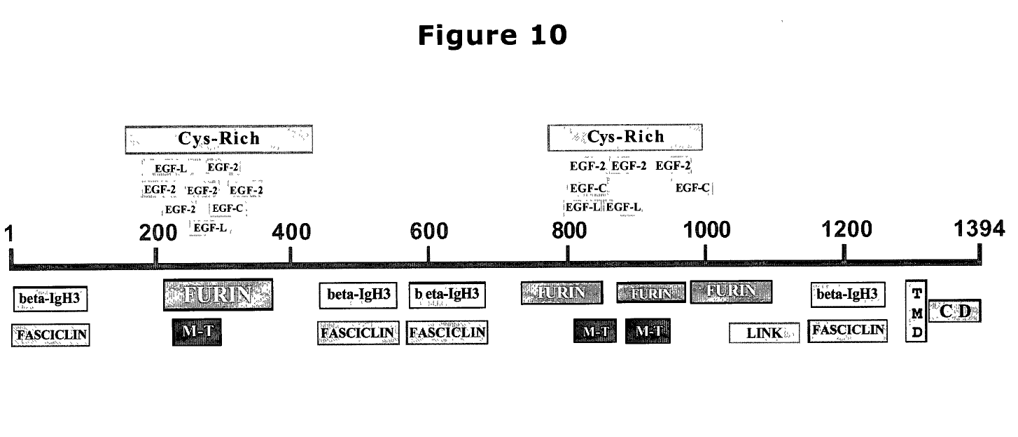

The human HARE is predicted to be a type I membrane protein (FIG. 10), with a large NH₂-terminal extracellular domain (>1300 amino acids), a single transmembrane domain (˜21 amino acids), and a small COOH-terminal cytoplasmic domain (˜72 amino acids). The predicted mass of the 1416 residue partial core protein determined here is 154,091 Da, and the pI is pH 5.91. The protein contains 17 potential N-glycosylation sites (-N-X-T/S-) in the extracellular domain. Twelve of these sites are identical with sites in the rat 175 kDa HARE (FIG. 11). An additional three nonclassical glycosylation sequons (-N-X-C-) are present in the human HARE, two of which are conserved with the rat HARE. An interesting feature of these Cys-containing sites is that glycosylation and participation of the Cys in a disulfide bond may be mutually exclusive (Miletich and Broze, ]. Biol. Chem. 265:11397 (1990)). The 190 kDa HARE extracellular domain has two cysteine-rich regions and multiple EGF-like, βIgH3, Furin, Metallothionein and Fasciclin domains, as well as DSL domains and one 93 amino acid Link (or XLink) domain near the membrane junction (Gly¹⁰⁶³-Tyr¹¹⁵⁵). Many of the programs such as Pfam-HMM, ScanProsite, SMART (Schultze et al, Proc. Natl. Acad. Sci. USA 95:5857 (1998)) or CD-Search identify domains that are only partial or weak matches and overlap with other domains. In particular the EGF-like domains show this characteristic (FIG. 10). Although the overall organization of all these above domains is very similar between the human and rat HARE proteins, the exact arrangement and number of each type of domain is not identical.

The human 190 kDa HARE and the rat 175 kDa HARE protein sequences are 78.1% identical, with a gap frequency of only 0.2% (using the SIM Alignment Program), over a region containing 1416 residues (FIG. 11). An additional ˜6.5% of the amino acid differences between the two proteins are conservative substitutions (e.g. R/K or S/T). Almost all of the cysteine residues within the extracellular domains of the two HARE proteins are absolutely conserved, which suggests that the two proteins have the same overall folding and organization of their polypeptide chains. The other HARE family members noted in FIG. 7 also share this extensive conservation of cysteine residues in their extracellular domains, as well as the same overall domain organization including the XLink domain and a single predicted transmembrane region. Unlike the rat protein, the human HARE has no cysteine residues in its transmembrane or cytoplasmic domains. The cytoplasmic domains of the two HARE proteins are less conserved (˜25% identical) than their transmembrane (˜76% identical) or extracellular domains (˜80% identical). Nonetheless, two candidate (pXXB motifs for targeting these receptors to coated pits are highly conserved: the human HARE YSYFRI¹³⁵⁰ and FQHF¹³⁶⁰ motifs differ by only one amino acid from the corresponding regions in the rat HARE cytoplasmic domain (FIG. 11).

Table III identifies several putative motifs from the human HARE protein that may be present in “HARE-like” proteins. Such “HARE-like” proteins have the ability to bind at least one of HA, chondroitin and chondroitin sulfate, and the “HARE-like” proteins comprise the LINK domain (SEQ ID NO:5) and at least one motif selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and sequences that are substantially identical to or only contain conserved or semi-conserved amino acid substitutions to the above-referenced sequences.

TABLE III

Putative Motifs of “HARE-like” Proteins

| | | Residues in |
|---|---|---|
| SEQ | | hHARE (from |
| ID | | SEQ ID |
| NO: | Amino Acid Sequence | NO:4) |
| 5 | GVFHLRSPLGQYKLTFDKAREACANEAATMATYNQL | G¹⁰⁶³ -Y¹¹⁵⁵ |
| | SYAQKAKYHLCSAGWLETGRVAYPTAFASQNCGSGV | |
| | VGIVDYGPRPNKSEMWDVFCY | |
| 6 | GTACETCTEGKYGIHCDQACSCVHGRCNQGPLGDGS | G²⁴⁵ -D²⁹³ |
| | CDCDVGWRGVHCD | |
| 7 | CKAGYTGDGIVCLEINPCLENHGGCDKNAECTQTGP | C³⁶⁵ -Q⁴⁰² |
| | NQ | |
| 8 | IDKLLSPKNLLITPKD | I⁵⁸⁵ -D⁶⁰⁰ |
| 9 | ALPAEQQDFLFNQDNKDKLK | A⁶⁵⁴ -K⁶⁷³ |
| 10 | CRIVQRELLFDLGVAYGIDCLLIDPTLGGRCDTFTT | C⁷²⁵ -D⁷⁶² |
| | FD | |
| 11 | DCQACPGGPDAPCNNRGVC | D⁸²³ -C⁸⁴¹ |
| 12 | CKCNTGFNGTACEMCWPGRFGPDC | C⁸⁵¹ -C⁸⁷⁴ |
| 13 | CSDHGQCDDGITGSGQCLCETGWT | C⁸⁷⁹ -T⁹⁰² |
| 14 | YEGDGITCTVVDFC | Y⁹³⁸ -C⁹⁵¹ |
| 15 | GGCAKVARCSQKGTKVSCSC | G⁹⁵⁶ -C⁹⁷⁵ |
| 16 | PCADGLNGGCHEHATC | P⁹⁹¹ -C¹⁰⁰⁶ |
| 17 | TGPGKHKCECKSHYVGDG | T¹⁰⁰⁹ -G¹⁰²⁶ |
| 18 | PIDRCLQDNGQCH | P¹⁰³⁵ -H¹⁰⁴⁷ |

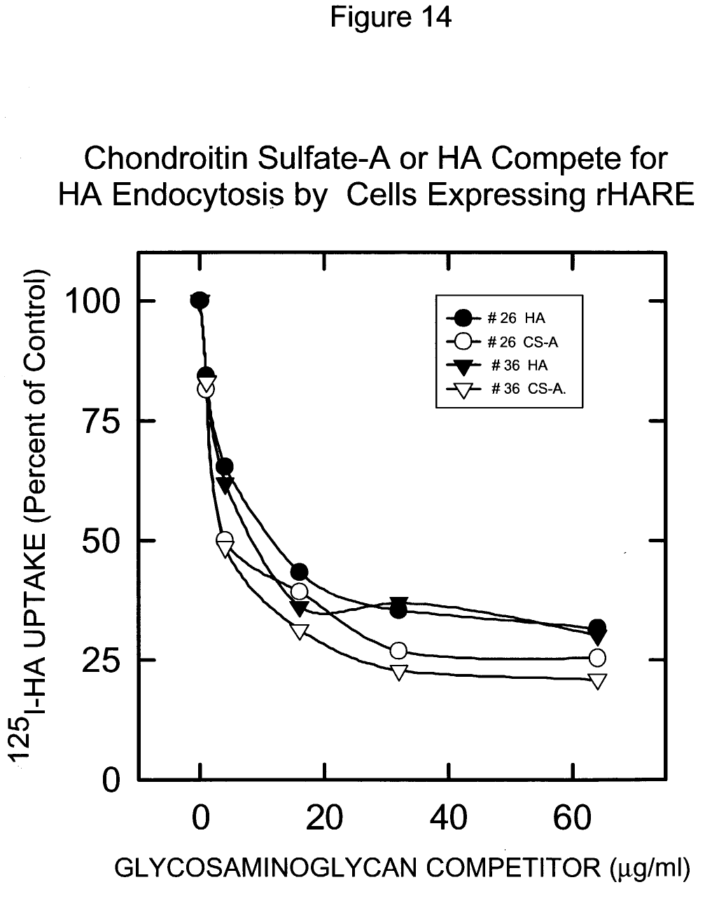

### Description of FIGS. 14-18

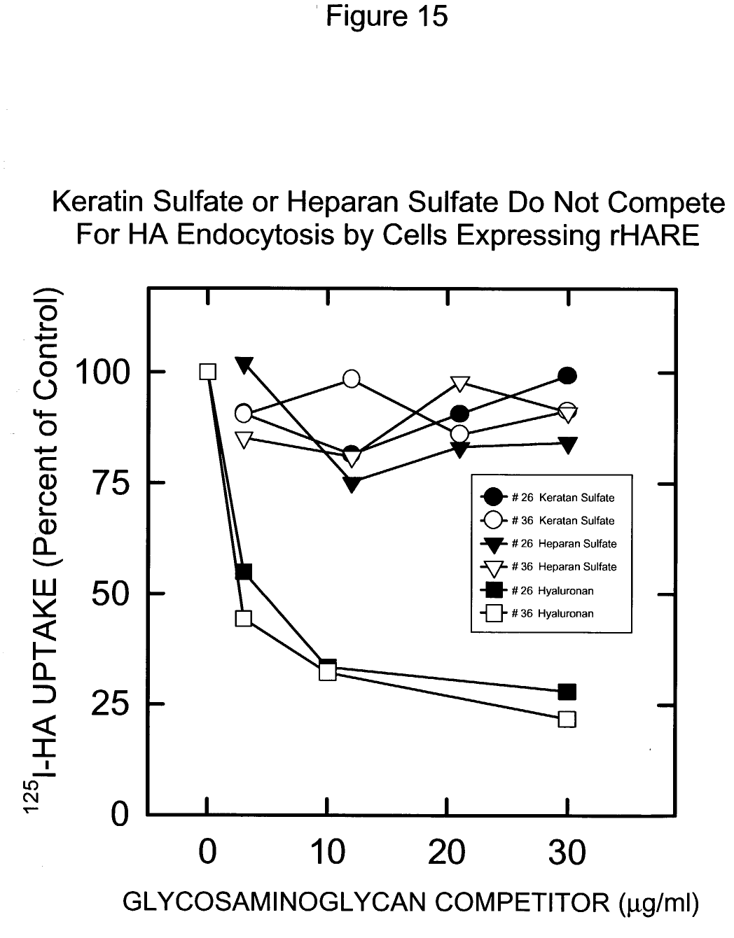

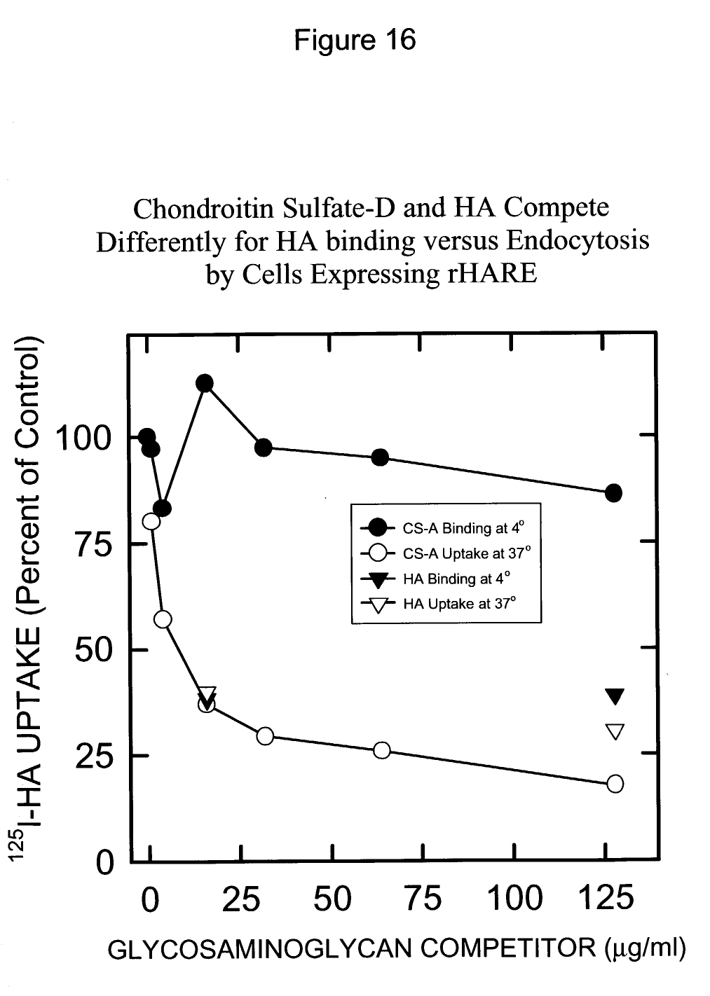

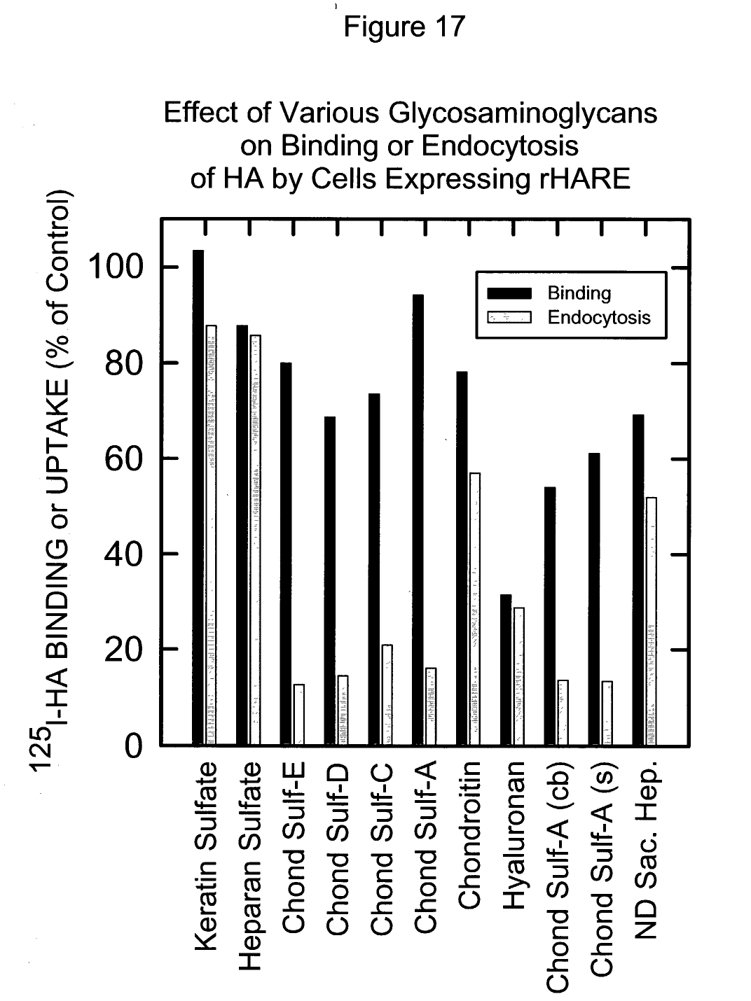

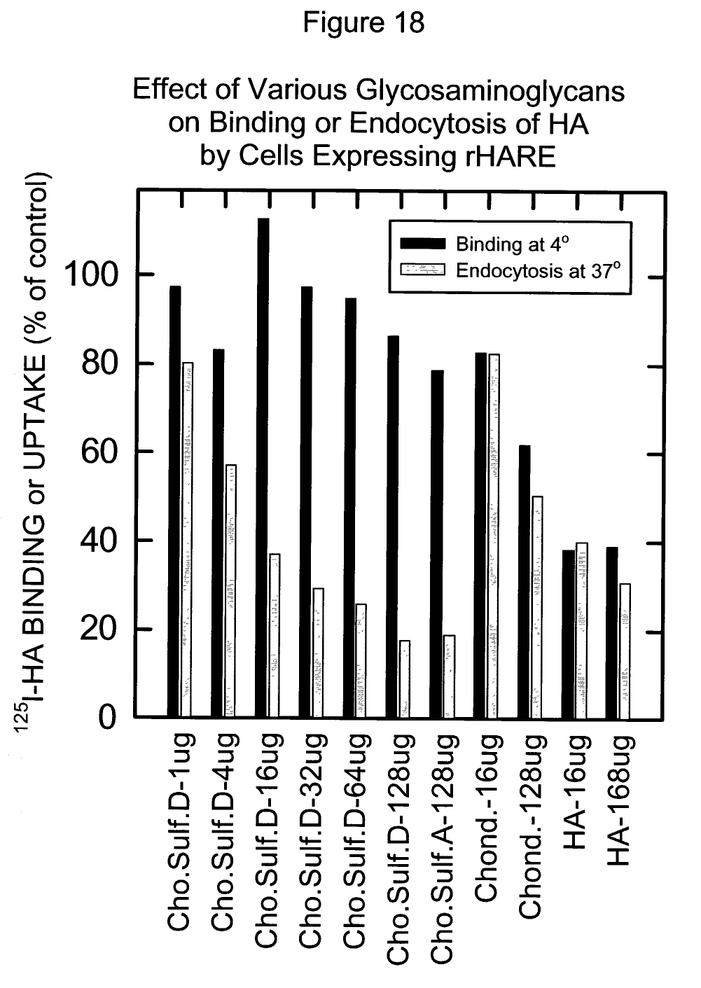

FIGS. 14-18 disclose the first data obtained on a cell line expressing only a single well-defined form of HARE. Experiments were performed with two independent clones of SK-Hep-1 cells, which were stably transfected with a cDNA encoding the rat HARE (rHARE); these cell lines are designated clones #26 and #36. The parent cell line does not express HARE and is unable to bind and endocytose HA efficiently. FIG. 14 shows that nonlabeled HA or chondroitin sulfate-A effectively compete for the ability of these cell lines to endocytose ¹²⁵I-HA. The glycosaminoglycans heparan sulfate and keratan sulfate were not effective as competitors, indicating that these molecules are not recognized by HARE (FIG. 15). Although both HA and chondroitin sulfate-A are internalized by HARE at 37° C., only HA is bound effectively by HARE at 4° C. (FIG. 16). This differential behavior with respect to binding at low temperature versus binding and internalization at higher temperature was also found with various other glycosaminoglycans (FIG. 17), including chondroitin sulfate-E, chondroitin sulfate-D (FIG. 18), chondroitin sulfate-C, as well as chondroitin sulfate-A from different sources (vendors such as Seigakaku, Calbiochem and Sigma). Many related glycosaminoglycans, including chondroitin (FIGS. 17 and 18) and N-desulfated and N-deacetylated heparin demonstrated the ability to bind to and be internalized by HARE. All of these results demonstrate that cells expressing HARE acquire the ability to bind to and internalize HA, chondroitin sulfate and chondroitin.

### Description of FIGS. 19-21

There is a large literature supporting the involvement of HA itself or hyaluronidases in cancer, particularly in the process of metastasis wherein malignant cells leave a primary tumor, migrate through multiple cell layers to enter and then leave the vasculature and ultimately enter a target tissue where they will establish a secondary tumor. In general the high mortality of cancers is not associated with the primary tumor but rather with the secondary metastases, which are very often found in liver, lymph nodes and bone marrow, the same tissues in which we have disclosed the presence of the HA Receptor for Endocytosis. Auvinen et al (Am. J. Pathol. 156:529 (2000)) showed a high correlation between HA expression levels, metastasis to lymph nodes and decreased survival of breast cancer patients. The very close link between metastasis and cellular synthesis of, and interactions with, HA indicates that HA can play a critical role in this process. For example, Simpson et al. (J. Biol. Chem. 276:17949 (2001)) demonstrated that tumor cells producing an HA coat are much more able to interact with and bind to bone marrow endothelial cells and that this interaction may be important in the cell homing process by which a malignant prostate cell is able to migrate to and establish itself in bone marrow. Similarly, Itano et al (Cancer Res. 59:2499 (1999)) showed that mutants of a mouse mammary carcinoma cell line that were unable to synthesize HA had a significantly decreased ability to metastasize in an animal model, but when transfected with a cDNA encoding HA synthase 1, these cells were rescued in their ability to make HA and to metastasize. Other studies support the idea that HA on the tumor cell or the endothelial cell can mediate cell adhesion, which is a critical step in metastasis, if the other cell has a cell surface component able to bind HA (Okada et al, Clin. Exp. Metastasis, 17:623 (1999)).

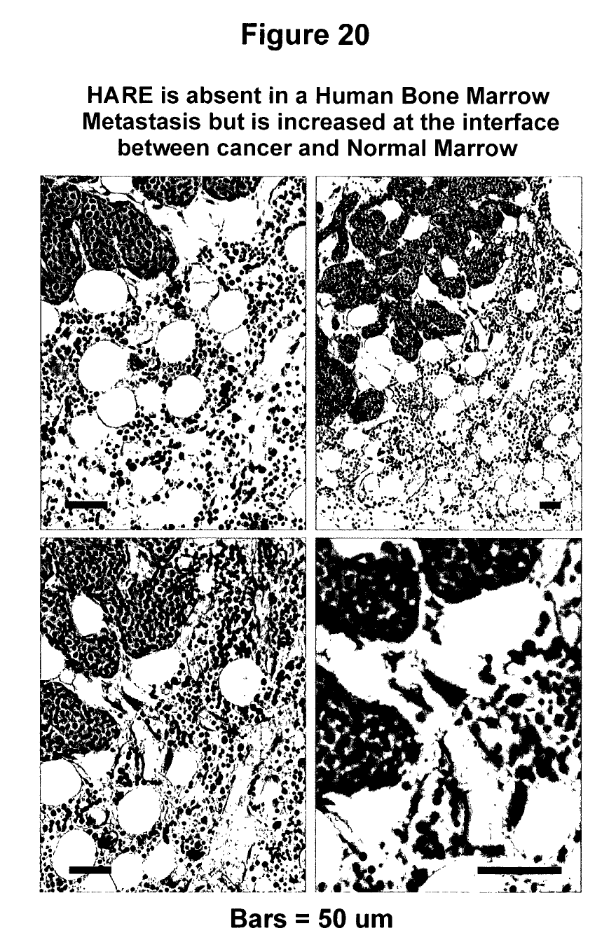

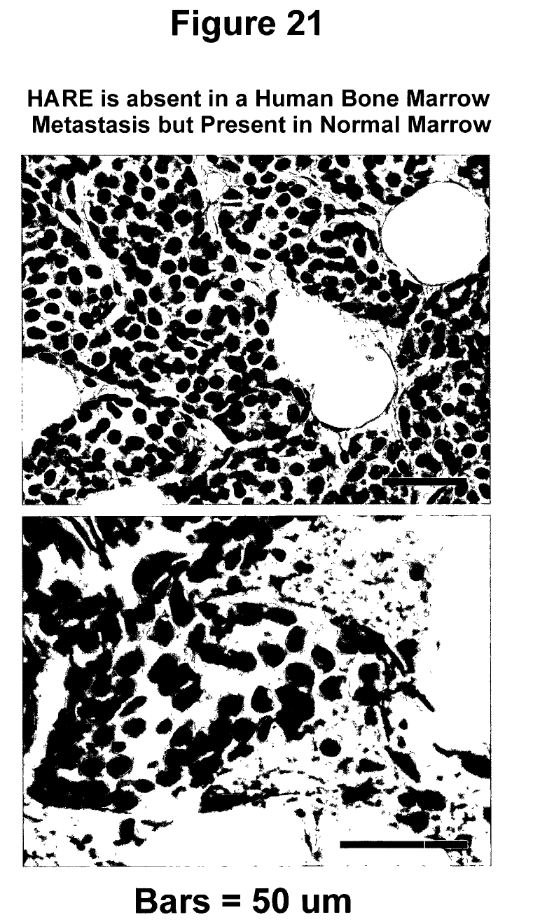

The immunocytochemical localization of human HARE in bone marrow, utilizing our specific monoclonal antibodies against HARE, demonstrates the expression of HARE in the sinusoidal endothelial cells of normal marrow (FIG. 19) in a female patient with primary ductal breast cancer. The control (lower right panel) using mouse serum rather than the anti-HARE mAbs shows no staining. The same patient had metastasis to the femoral head, and FIG. 20 shows that the HARE expression appears normal in regions of marrow adjacent to the cancer (the tumor is to the upper left in all four panels). The cancer cells are not stained for HARE, indicating it is absent in the tumor. In areas immediately adjacent to the cancer, the expression of HARE in the human bone marrow endothelial (HBME) cells appears to be enhanced. The control (upper left panel) using mouse serum rather than the anti-HARE mAbs shows no staining. FIG. 21 shows even higher magnification views of the cancer cells (top panel) and bone marrow endothelial cells (bottom panel).

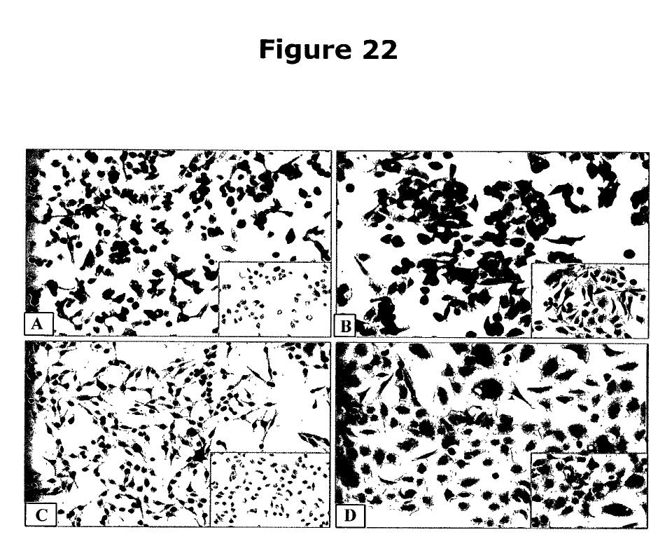

### Description of FIGS. 22-26

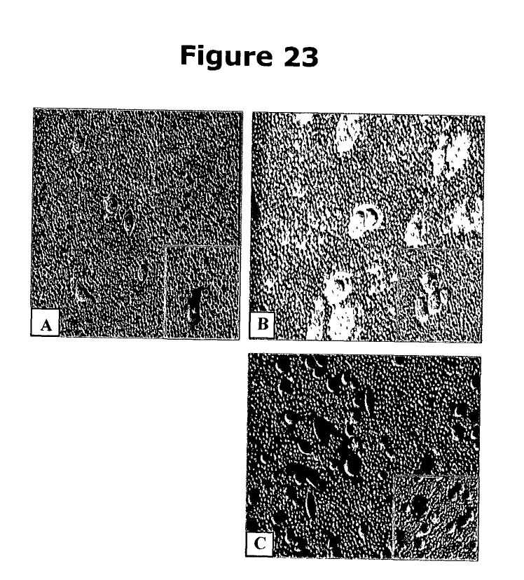

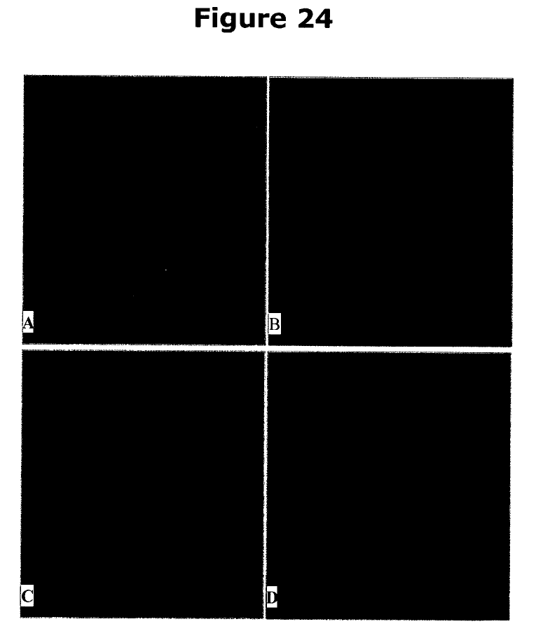

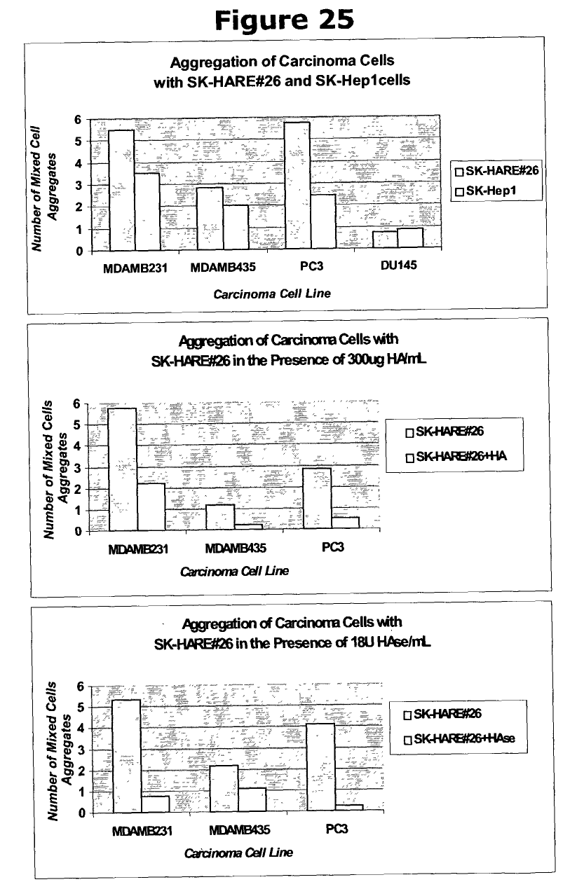

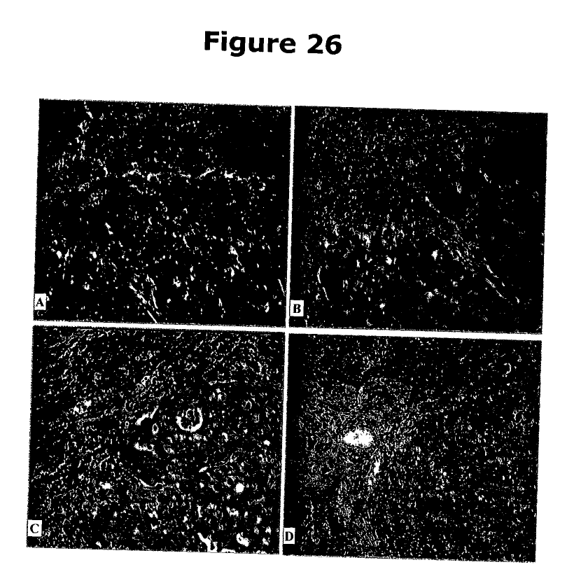

Cell-associated HA has been increasingly associated with carcinoma cell metastasis. Metastasis of some cancer cells to specific tissues could involve specific binding interactions between HA on the tumor cell surface and HA receptors on particular cell types in the target tissue. This possibility was investigated using an in-vitro model of HA mediated carcinoma cell adhesion. The metastatic human breast carcinoma cell line MDA-MB-231 shows increased cell surface HA (based on a particle exclusion assay or staining with a biotinylated-HA binding protein) compared to the metastatic human breast carcinoma cell line MDA-MD-435 (FIGS. 22 and 23). Similarly, the human metastatic prostate cancer cell line PC3 has increased peri-cellular HA compared to the less metastatic DU145 human prostate cancer cell line. Stably transfected SK-Hep-1 cells expressing the HARE (SK-HARE cells) are able to internalize and accumulate fluorescent-HA (FIG. 24). MDA-MB-231 and the PC3 cells, both of which express high levels of HA, show increased aggregation (FIG. 25) with SK-HARE cells compared to control SK-Hep1 cells (not expressing HARE). The MDA-MB-435 and DU145 carcinoma cells, which express little or no cell surface HA, do not form similar aggregates. The observed cell-cell adhesion is mediated by the interaction between HA and HARE, because this adhesion is blocked by excess free HA or by pretreatment of the tumor cells with hyaluronidase. The results demonstrate that HARE, which is highly expressed in liver, lymph node and bone marrow (very common sites of adenocarcinoma metastasis), could be a “homing receptor” that mediates the capture and localization of tumor cells expressing cell surface HA. Tissue sections from lymph nodes containing metastatic breast carcinoma show tumor cells that contain cell surface HA have apparently arrested in the lymph node at sites of HARE expression (FIG. 26).

Carcinoma metastasis requires specific biochemical interactions at the metastatic site between the tumor cells and endothelium to mediate adhesion and tumor cell arrest. In breast carcinoma, subsets of tumor cells undergo phenotype changes allowing them to accomplish all steps in the metastatic cascade. This includes detachment from the primary tumor, invasion of tissue, entry into lymphatics/vasculature, dissemination and avoidance of host defense, arrest at a distant site, exit from the circulation and finally proliferation at the secondary site (Seraj et al., Cancer Res. 60:2764 (2000)). Tumor cell arrest in the metastatic site can be facilitated by receptor-ligand interactions. A recent report indicates that hyaluronan (HA) on prostate carcinoma cell surfaces is important for adhesion of prostate carcinoma cells to bone marrow endothelium (Lehr et al., J. Natl. Cancer Inst. 90:118 (1998); Simpson et al., J. Biol. Chem. 276:17949 (2001)). The HBME cell surface molecule responsible for this adhesion has not been identified. Candidate HA binding proteins would include CD44 (Simpson et al., J. Biol. Chem. 276:17949 (2001)), the Receptor for HA mediated motility (RHAMM) (Lokeswar et al., J. Biol. Chem. 275:641 (2000)), the lymph vessel endothelial specific HA receptor (LYVE-1) (Banerji et al., J. Cell Biol. 144:789 (1999)) and HARE (Zhou et al., J. Biol. Chem. 275:733 (2000)). Incubation of HBME cells with CD44 blocking antibodies failed to inhibit HA-mediated prostate cancer cell adhesion, making CD44 a less likely candidate (Simpson et al., J. Biol. Chem. 276:17949 (2001)). RHAMM has not been described in HBME cells, although it can be involved in lung metastasis (Lokeswar et al., J. Biol.Chem. 275:641 (2000)). LYVE-1 mRNA was detected in bone marrow; however, bone marrow protein expression was not confirmed by immunohistochemistry (Banerji et al., J. Cell Biol. 144:789 (1999)). HARE is expressed in spleen, liver, lymph node and bone marrow, the latter three organs being common sites of carcinoma metastasis.

### Materials and Methods for FIGS. 22-26

Cell culture and reagents. MDA-MB-231 and MDA-MB-435 metastatic breast carcinoma cells were maintained in DMEM/Ham's F12 with 5% FBS, and split at 80-90% confluence with 0.05% trypsin. SK-HARE and SK-Hep1 cells were maintained in DMEM with 5% FBS, and split at 80-90% confluence with 0.05% trypsin. Medium for the SK-HARE cells also contained 500 μg/ml geneticin. PC3 and DU145 prostate cancer cells were maintained in F12K with 7% FBS and EMEM with 10% FBS respectively, split at 80-90% confluence with 0.25% trypsin. All cells were maintained at 37° C. and 5% CO₂ and grown in the absence of antibiotics.

Demonstration of tumor and tumor cell associated HA. Tumor cell associated HA was directly demonstrated by peroxidase staining using a biotinylated HA binding probe (Seikagau, Japan) following the manufacturers protocol with and without a Streptomyces hyaluronidase pretreatment to assess specificity. Color was developed with 2% CV/VS aminoethylcarbazole according to the manufacturer instructions, followed by counterstaining with hematoxylin. Tumor cell-associated HA was also indirectly demonstrated in cultured cells with a particle exclusion assay. Glutaraldehyde-fixed sheep red blood cells in PBS/1% BSA were added to cultures of subconfluent carcinoma cells, allowed to settle for 15 min and then observed under phase contrast microscopy. Specificity of the assay was shown by hyaluronidase preteratment of tumor cells.

Assay for functional HARE. Hyaluronan hexylamine derivative (Raja et al., Analytical Biochem. 139:168 (1984)) was reacted with rhodamine green succinimidyl ester (Molecular Probes, Eugene Oreg.) according to manufacturer's instructions for coupling proteins, quenched, and purified from reactants by gel filtration. The SK-Hep1 cells and SK-HARE tranfectants were incubated at 37° C. with 20 μg/ml of rhodamine green-HA (RG-HA) with or without a 50-fold excess of unlabeled HA for 6 hours.

Cell aggregation assay. SK-HARE and SK-Hep1 cells were labeled with the red fluorescent dye 1,1′-dioctadecyl-3,3,3,3′-tetramethylindocarbocyanine perchlorate (Dil C-18), (Molecular Probes, Eugene, Oreg.) and carcinoma cells were labeled with the green fluorescent dye calcein AM (Molecular Probes) for 40 min, and the labeled cells were harvested from culture dishes by mild trypsinization. Approximately 10⁵ SK-HARE or SK-Hep1 cells were mixed with 10⁵ carcinoma cells and allowed to aggregate for 30 min at 37° C. with gentle mixing. The number of co-aggregates (containing both red and green cells) was assessed after 25 min in a semi-quantitative manner by counting the distribution of cells in aggregates in 10 separate fields at low magnification (100×) using epi-fluorescence microscopy.

Inhibition of cell aggregation. Cell suspensions labeled with calcein AM were pre-incubated with 16 U/mL Streptomyces hyaluronidase for 1 hour before performing the aggregation assay and hyaluronidase was maintained throughout the assay to remove any HA synthesized by the cells during the assay. Dil C-16-labeled SK-HARE cells were also pre-incubated with 300 μg/ml of exogenous HA (MW˜44,000) which was maintained throughout the aggregation assay to interact with HARE and block its ability to bind HA on the tumor cell surfaces.

Human Metastatic Breast Carcinoma. Cases of breast ductal carcinoma were identified by computer search of the surgical pathology database at the University of Rochester following approval from the Institutional Research Subjects Review Board. The original hematoxylin and eosin stained sections were reviewed and tissue blocks selected for study included the primary breast carcinoma as well as a representative axillary lymph node. The tissue was fixed in 10% neutral buffered formalin and paraffin embedded at the time of original surgery using routine methods. Sections (5 μm) were cut and allowed to dry overnight at 60° C. Paraffin was removed through a series of xylene and alcohol washes, and endogenous peroxidase activity was quenched with 3% hydrogen peroxide. The slides were then subjected to antigen retrieval. Visualization using the anti-HARE antibody mAb#30, and the nonimmune IgG controls, required pepsin digestion for antigen retrieval. The slides were placed in a prewarmed solution of 16 mg of pepsin in 50 ml of 0.1N HCL and incubated at 37° C. for 15 min. The slides for biotinylated-HA binding protein required no antigen retrieval, although a hyaluronidase digestion was employed to assess specificity. The slides were washed with PBS and incubated with the appropriate primary antibody diluted in PBS at room temperature for 60 min. After washing in PBS the slides were treated with biotinylated horse anti-mouse IgG (1:200) for 30 min at room temperature. The slides were then washed with PBS, incubated with streptavidin peroxidase (1:1000), washed once with PBS and once with distilled water and color development was achieved by incubating with 2.0% v/v aminoethylcarbazole and hydrogen peroxide for 5 min according to the manufacture's instructions (ScyTek, Utah). Hematoxylin was used for counterstaining. Slides were viewed with an Olympus BH-2 light microscope equipped with an Olympus 35mm camera for photomicroscopy.

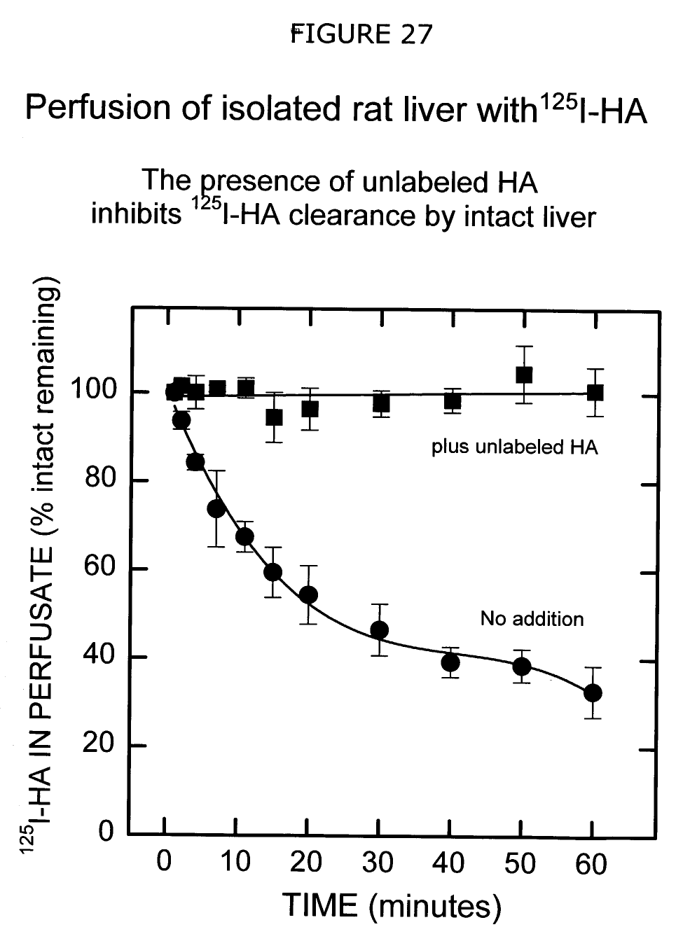

### Description of FIGS. 27-29

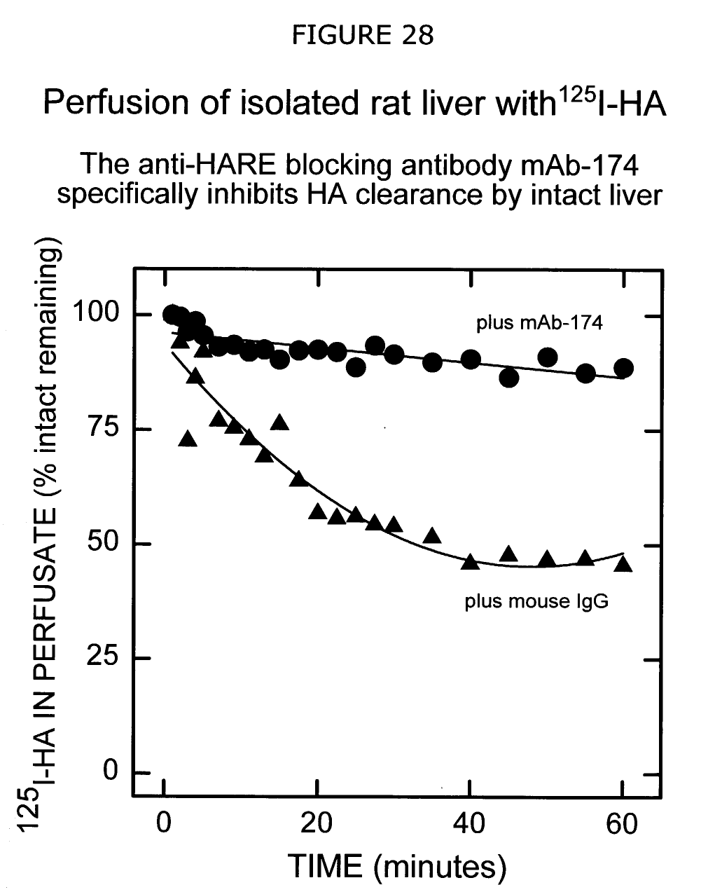

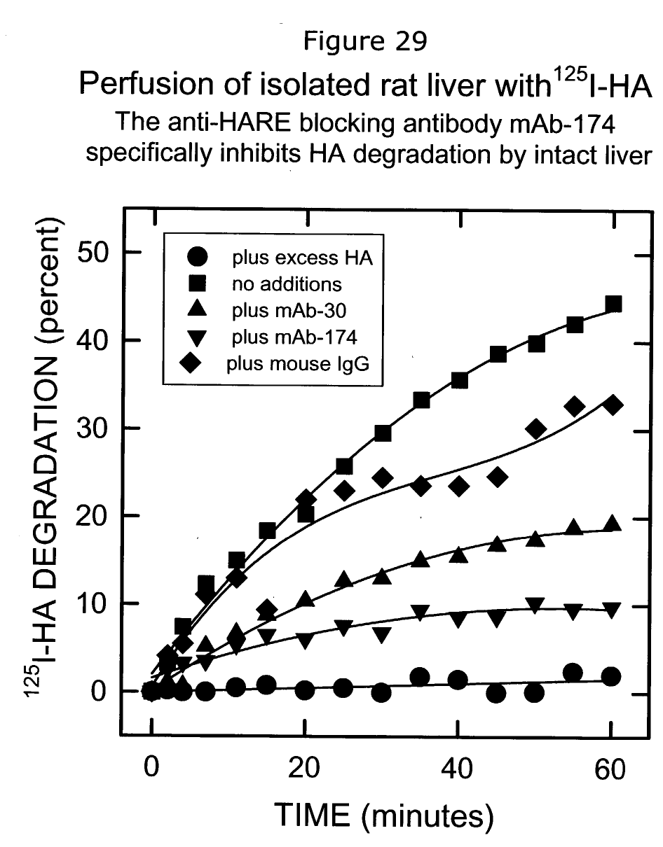

FIGS. 27 and 28 are continuous perfusion (with recirculation) experiments with isolated rat liver that demonstrate that excess unlabeled HA and the anti-HARE blocking antibody mAb-174 specifically inhibit HA clearance by intact liver. FIG. 29 demonstrates that excess unlabeled HA, mAb-30 and mAb-174 specifically inhibit HA degradation by intact liver.

In FIG. 27, isolated rat liver is reperfused with continuous recirculation with ¹²⁵I-HA, and the uptake of ¹²⁵I-HA by the rat liver (labeled as “No addition”) can be observed over time. The addition of unlabeled HA competitively inhibits this uptake, demonstrating that the clearance of ¹²⁵I-HA is due to a receptor that specifically recognizes HA.

In FIG. 28, the anti-HARE blocking antibody mAb-174 also specifically inhibits ¹²⁵I-HA clearance by intact liver, while the addition of mouse IgG does not affect ¹²⁵I-HA uptake by the liver. This demonstrates that the specific receptor responsible for the clearance of ¹²⁵I-HA is HARE. These results are consistent with the findings of Laurent and co-workers that liver is the major site of HA clearance from the blood.

In FIG. 29, isolated rat liver is reperfused with ¹²⁵I-HA, and the degradation of ¹²⁵I-HA by the rat liver (labeled as “no additions”) is observed. The addition of excess HA completely inhibits such degradation, while mAb-30 and mAb-174 also inhibit degradation of ¹²⁵I-HA. The addition of mouse IgG has very little affect of the degradation of ¹²⁵I-HA.

Materials and Methods for FIGS. 27-29

Materials. ¹²⁵I-HA was prepared using a unique alkylamine derivative of HA (oligosaccharides of Mᵣ˜70,000) as previously described by Raja, et al (1984). Male Sprague-Dawley rats (200 g) were from Charles River Labs. BSA Fraction V was from Intergen Co. The preparation of mouse mAbs against the rat HARE was described by Zhou et al (2000). All other chemical and reagents were from Sigma Chemical Co.

Liver perfusion. Rat livers were removed and perfused ex vivo with Buffer 1 (142 mM NaCl, 6.7 mM KCl, and 10 mM HEPES, pH 7.4) for 8-10 min at ˜35° C. The liver was then perfused by recirculation with 60 ml of medium (GIBCO cat. # 41100) supplemented with 60 mM HEPES, pH 7.4 and 0.1% (w/v) BSA containing 0.25 μg/ml of ¹²⁵I-HA for up to 60 min at ˜35° C. Samples (300 μl) of perfusate were taken at the noted times and divided into 50 μl portions for determination of radioactivity (in duplicate) or degradation (in triplicate). Competitor unlabeled HA (50 μg/ml), purified mAb IgG or mouse IgG (1-5 μg/ml) were added to the perfusion medium containing the ¹²⁵I-HA and mixed well before starting the perfusion. Prior to exposure to the ¹²⁵I-HA, the livers were pre-perfused for 3-25 min with the same concentration of HA or IgG in medium supplemented with 50 μg/ml goat IgG (Sigma cat #I-5256) at ˜35° C.

Degradation of ¹²⁵I-HA. Degradation of ¹²⁵I-HA was measured by a CPC (cetylpyridinium chloride) precipitation assay. Fifty μl portions of perfusion medium containing ¹²⁵I-HA were added (in triplicate) to 250 μl of 1 mg/ml HA (as a carrier) in water in microfuge tubes. Then 300 μl of 6% CPC (in d₂H₂O) was added and the tubes mixed by vortexing. After 10 min at room temperature, the samples were centrifuged in an Eppendorf model 5417 microfuge at room temperature for 5 min at 9000 rpm. Samples (300 μl) were taken, and the remaining supernatants were removed by aspiration. The tips of the tubes were then cut off, put in a gamma counter tube and radioactivity in these and the supernatant samples were determined. Degradation was calculated as the fraction of total radioactivity in each sample that was soluble (non-precipitable). Note that ˜20 to 30% of the radioactivity was not precipitable at the beginning of the experiments.

### Description of FIG. 31

The mRNA, partial cDNA, amino acid sequence and mAb reactivity data are all consistent with the hypothesis that there is a precursor relationship among the 260 and 230 kDa subunits of the 300-kDa HARE and the 175-kDa HARE protein. To test this possibility, the reactivity of these three HARE proteins with two different polyclonal anti-peptide antibodies was examined. One Ab was raised against a sequence within the rat 175-kDa protein (PKCPLKSKGVKK⁷⁷³), and the other Ab was raised against a 16-amino acid putative coding region (TVLVPSRRAFEDMDQNK) that begins 107 amino acids upstream of the SLP . . . sequence identified as the amino-terminal start of the purified rat 175-kDa protein. There was no prior information about whether this putative protein region is expressed. However, if all three HARE proteins are derived from a larger precursor, the prediction was that the former Ab should recognize all three proteins, whereas the latter Ab would recognize only the two larger proteins but not the 175-kDa protein. This was the result obtained (FIG. 31), which strongly supports the conclusion that the 175-kDa HARE is indeed derived from one of the larger HARE proteins of the 300-kDa HARE.

### Description of FIG. 32

FIG. 32 demonstrates that the core proteins of the human 190 kDa HARE and the rat 175 kDa HARE are essentially the same size after removal of N-linked oligosaccharides. Purified rat 175 kDa and human 190 kDa HARE were denatured, de-N-glycosylated and then detected using anti-HARE mAbs against the rat 175 kDa HARE. After removal of the N-linked oligosaccharide, both core proteins migrate at the same position, marked by the dashed arrow, indicating that both proteins are essentially identical in size. The apparently larger size of the human 190 kDa HARE relative to the rat HARE is due to the presence of either more or larger oligosaccharides.

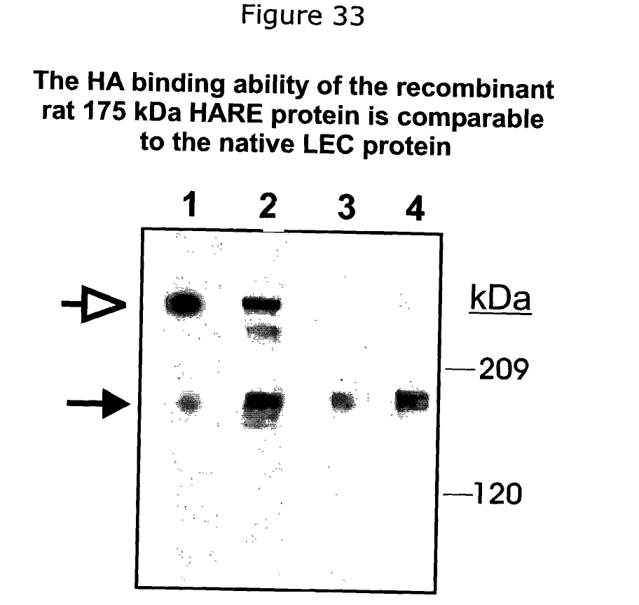

### Description of FIGS. 33-36

To further confirm that the bone fide cDNA for the rat 175-kDa HARE had been cloned, HA binding and internalization studies were performed using transfected COS-7 or SK-Hep-1 cells expressing the 175-kDa protein. Since there is no natural mRNA directly coding for the 175-kDa HARE protein, an artificial cDNA that encodes the ORF for the 175-kDa HARE fused at the 5′ end to a short region of the Ig κ-light chain sequence containing a start codon and a membrane insertion signal or leader sequence was constructed. Transient transfection of this cDNA into COS-7 cells yielded a protein of the expected size that was recognized in Western blots by the specific anti-HARE mAbs and that bound ¹²⁵I-HA specifically in the ligand blot assay.

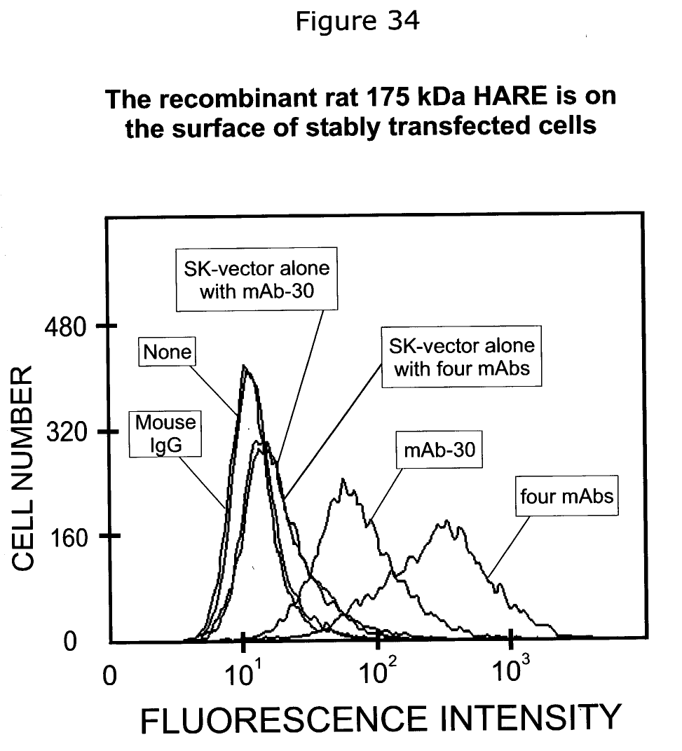

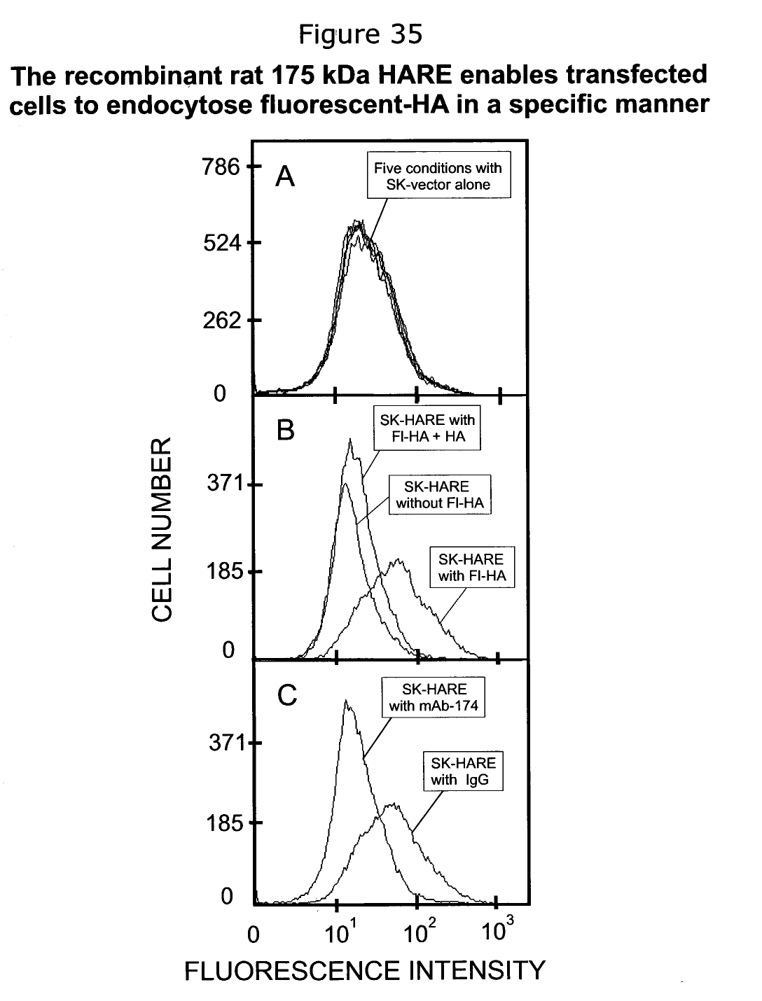

This p175 HARE-κ vector was then used to generate stable cell lines expressing HARE after antibiotic selection of transfected SK-Hep-1 cells. This cell line was chosen because it does not express any detectable endogenous HA receptors capable of specific ¹²⁵I-HA binding or endocytosis, and does not show reactivity with the anti-HARE mAbs in Western blots. Seven independent clones were selected, all of which had essentially identical characteristics with respect to 175-kDa HARE expression and function. The recombinant 175-kDa HARE expressed by these cells and the purified rat LEC protein were essentially identical in their ability to bind ¹²⁵I-HA in the ligand blot assay (FIG. 33). FACS analysis showed that the recombinant HARE protein was localized to the cell surface (FIG. 34). Specific mAbs against the 175-kDa HARE bound to cells expressing HARE, but not to SK-Hep-1 parental cells or cells transfected with vector alone. The internalization of fluorescent-HA by SK-HARE cells was specific as judged by its competition with unlabeled HA (FIG. 35B), its inhibition by mAb-174 (FIG. 35C), and the lack of uptake by SK-Hep-1 cells or cells transfected with vector alone (FIG. 35A).

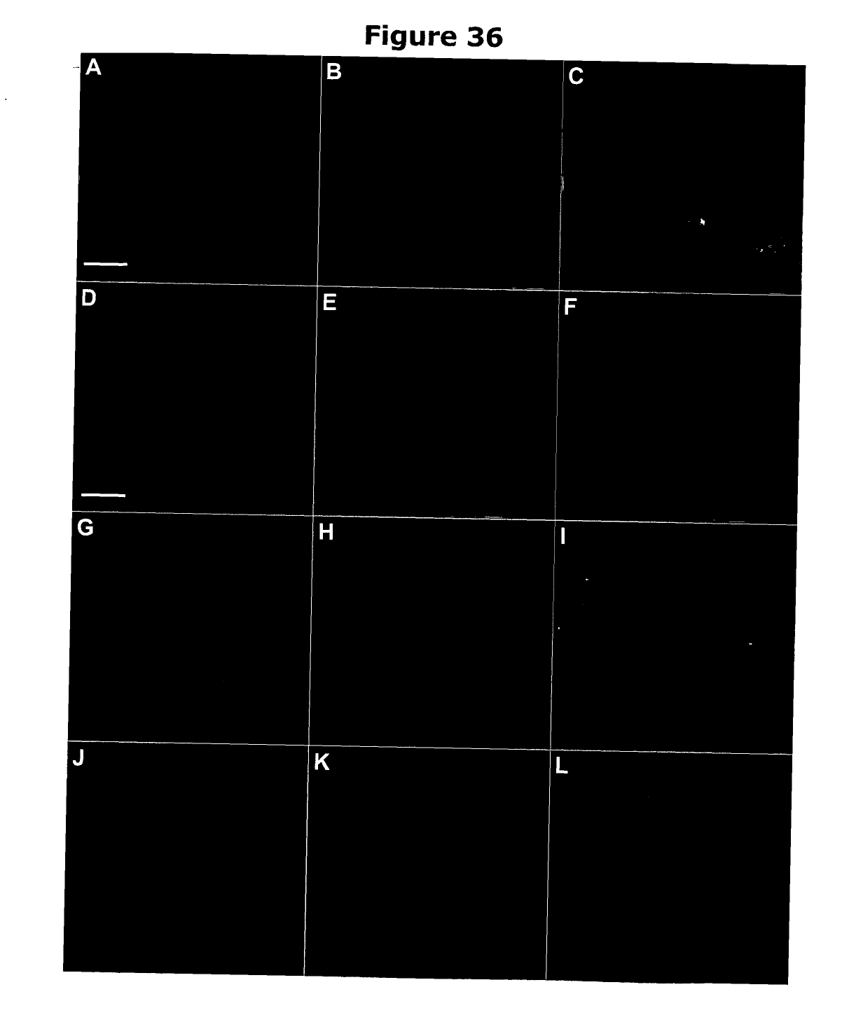

Confocal fluorescence microscopy was then used to assess the cellular distribution of HARE and internalized HA in SK-HARE cells (FIG. 36). As expected for a recycling receptor mediating endocytosis via coated pits, much of the cellular clathrin was colocalized with HARE (FIGS. 36A-C), whereas most of the intracellular HARE staining was not present in clathrin-containing compartments, which is typical for an endocytic, recycling receptor. HARE was not targeted to lysosomes as a consequence of mediating HA uptake (FIGS. 36D-F), although internalized HA was delivered to lysosomes as assessed by its co-localization with the Lysotracker dye (FIGS. 36G-I). The internalization of fl-HA was virtually eliminated by a large excess of unlabeled HA (FIG. 36J). A variety of controls showed no significant fluorescence, including SK-HARE cells treated with mouse or rabbit IgG (FIG. 36K), and SK-Hep-1 cells or cells transfected with vector alone (FIG. 36L) incubated with fl-HA.

### DISCUSSION

HA was discovered and named over 67 years ago by Meyer and Palmer (J. Biol. Chem. 107:629 (1934)), and then shown by many other investigators to be a common, ubiquitous, component of essentially all ECMs in vertebrates. HA is the only glycosaminoglycan that is not sulfated and not covalently attached to a core protein. It is a linear polymer composed of the repeating disaccharide unit 2-deoxy, 2-acetamido-D-glucopyranosyl-β(1,4)-D-glucuronopyranosyl-β(1,3) (Laurent and Fraser, Degradation of Bioactive Substances: Physiology and Pathophysiology, 249, CRC Press, Boca Raton, Fla. (1991); Laurent and Fraser, FASEB J. 6:2397 (1992)). The molecular weight of native HA can be as high as 10⁷, which is up to 1,000-times the size of other glycosaminoglycan chains attached to proteoglycans. The physical characteristics of HA solutions, particularly their rheologic properties and viscoelasticity, are ideally suited for the role of HA in specialized ECMs of skin, cartilage, and fluids such as in the vitreous humor of eye and the synovium of joints.

Although its structure is simple, HA influences many cell functions and behaviors, including cell migration, differentiation, and phagocytosis (Evered and Whelan, The Biology of Hyaluronan, 143:1 (1989); Laurent and Fraser, FASEB J. 6:2397 (1992); Knudson and Knudson, FASEB J. 7:1233 (1993); Toole, J. Intern. Med. 242:35 (1997); Abatangelo and Weigel, New Frontiers in Medical Sciences: Redefining Hyaluronan, Elsevier Science BV., Amsterdam (2000); Turley, Cancer Metastasis Rev. 11:21 (1992)). HA is an important molecule in development (Toole, J. Intern. Med. 242:35 (1997); Gakunga et al, Devel. 124:3987 (1997)), wound healing (Iocona et al, J. Surg. Res. 76:111 (1998); Burd et al, Br. J. Plast. Surg. 44:579 (1991); Weigel et al, J. Theoret. Biol. 119:219 (1986); Chen and Abatangelo, Wound Repair Regen. 7:79 (1999)), angiogenesis (West et al, Science, 14:1324 (1985); Deed et al, Int J. Cancer, 71:251 (1997); Rahmanian et al, Exp. Cell Res. 237:223 (1997)), and tumor growth and metastasis (Zhou et al, J. Biol. Chem. 276: in press (2000); Csoka et al Invasion Metastasis, 17:297 (1997); Delpech et al, J. Intern. Med. 242:41 (1997)). For example, the ability of HA to form large aggregates by binding to ECM proteoglycans, such as aggrecan and perlecan, is necessary for normal tissue differentiation (Vertel et al, Biochem J. 301:211 (1994); Handler et al, Dev. Dyn. 210:130 (1997)).

Previously, most investigators believed that the physiological function of HA in the ECM was only structural or physical. However, HA is now recognized as a pharmacologically active signaling molecule, in addition to an ECM structural component. Numerous cell types respond physiologically to HA of different sizes. In particular, small, but not large, HA stimulates angiogenesis (West et al, Science, 14:1324 (1985); Deed et al, Int J. Cancer, 71:251 (1997); Rahmanian et al, Exp. Cell Res. 237:223 (1997)) and small, not large, HA stimulates activated macrophages to induce the expression of a large number of genes (Horton et al, J. Biol. Chem. 273:35088 (1998); Horton et al, Am. J. Physiol. Lung Cell Mol. Physiol. 279:707 (2000)). Similarly, only small HA induces the expression of NO synthase in Kupffer cells and LECs, but not in stellate cells or hepatocytes (Rockey et al. Hepatol. 27:86 (1998)). Although most investigators presume that specific cell surface receptors in these sensitive cell types bind these small HA fragments and then mediate the stimulation of intracellular signal cascades, no such receptor has yet been identified.

There are currently about four known types of HA-binding proteins or hyaladherins (Toole. Curr. Opin. Cell Biol. 2:839 (1990)): enzymes, components of the ECM, cell surface receptors and soluble plasma or intracellular molecules. Cell surface HA receptors that have been characterized to date include CD44, LYVE-1, CD168 (formerly designated RHAMM), ICAM-1, and HARE. A scavenger receptor able to bind and internalize HA may also be present in liver (McCourt et al. Hepatol. 30:1276 (1999)). HARE is distinct from all the other cell surface receptors with specificity for HA because it is an endocytic, recycling receptor that mediates the rapid and efficient endocytosis of HA via the clathrin-coated pit pathway. CD168 is found on the surface of, and inside, many cell types and can mediate a cell migration response to HA (Turley et al. Blood, 81:446 (1993); Hofmann et al, J. Cell Sci. 111:1673 (1998)). The CD44 family of transmembrane glycoproteins is found in hemopoietic cells, epithelial and endothelial cells, lymphocytes and many cancer cells (Lesley et al. Adv. Immun. 54:271 (1993)) and has structural homology to cartilage link protein (Bajorath et al. J. Biol. Chem. 273:338 (1998)).

LYVE-1, a member of the CD44 family, is localized to lymphatic vessel endothelial cells in many tissues, but is not present in blood vessels (Banerji et al. J. Cell Biol. 144:789 (1999)). Preliminary results indicate that LYVE-1 and HARE have distinct, non-overlapping distributions within various lymphatic tissues. HARE is expressed in the sinusoids of liver and lymphatic tissues (Zhou et al. J. Biol. Chem. 275:37733 (2000)), which is a localization well suited to keep a very low level of systemic HA (i.e. HA that is not associated with an ECM). Liver, spleen and lymph node express large amounts of HARE for this purpose. ICAM-1 is an adhesion molecule on the cell surface that binds HA (Hayflick et al, Immunol. Res. 17:313 (1998)). Some confusion may still exist regarding ICAM-1 because several studies have appeared (Gustafson et al, Glycoconj. J. 12:350 (1995); Fuxe et al, Brain Res. 736:329 (1996)) that were based on the incorrect identification of ICAM-1 as the endocytic HA receptor in LECs (McCourt et al, J. Biol. Chem. 269:30081 (1994)). This misidentification was later acknowledged to be an artifact (McCourt and Gustafson, Int. J. Biochem. Cell Biol. 29:1179 (1997)), but the erroneous report was not withdrawn.

Because it is non-immunogenic and has special viscoelastic and Theological properties in solution, HA is used in many clinical applications, and its medical uses are growing rapidly. For example, ophthalmic surgeons worldwide routinely use sterile solutions of pure, pyrogen-free, high molecular weight HA in numerous procedures (Goa and Benfield, Drugs, 47:536 (1994); Panay and Lower, Curr. Opin. Obstet. Gynecol. 11:379 (1999)). HA is ideally suited for such uses, since it is a natural ocular component, and its physical properties keep the eyeball from collapsing. Many patients with osteoarthritis or rheumatoid arthritis now experience significant improvement after receiving intra-articular injections of HA (Pelletier and Martel-Pelletier. J. Rheumatol. 20:19 (1993)). Laurent et al. (Arch. Otolaryngol. Head Neck Surg. 114:1435 (1988)) have also used HA to heal perforated tympanic membranes, which then restores hearing. HA has been used topically to reduce postoperative pericardial adhesions and as an aerosol to prevent elastase-mediated injury in pulmonary emphysema (Cantor et al. Proc. Soc. Exp. Biol. Med. 217:471 (1998)). HA is also used as a drug delivery vehicle (Illum et al. J. Control Release, 29:133 (1994); Luo et al. J. Control Release, 69:169 (2000)). Because of its use in such a wide array of medical applications, it is critical that we understand the biological effects of exogenously administered HA and how its turnover and clearance from the body is regulated.

Clearance of the endogenous circulating HA from lymph and blood is also likely to be very important for normal health, because the viscosity of these fluids would rapidly increase to dangerous levels if the concentration of HA was allowed to accumulate, particularly if it was of high molecular weight as found in lymph fluid (more than about 10⁶). For example, one can readily envision the difficulty of erythrocyte flow through tiny capillaries under conditions of high viscosity. The 175/190 kDa and 300/315 kDa HARE proteins are two HA isoreceptors for endocytosis present in mammalian liver, spleen and lymph node. The two HA isoreceptors may be necessary to mediate HA uptake and degradation in mammals because of the extremely broad range of HA molecular masses present in tissues throughout the body. The two isoreceptors could have different preferences for the size of the HA with which they interact. Presumably the smaller HARE would interact with smaller HA and the larger HARE with larger HA.

The present results show that the rat 175 kDa HARE is a bone fide endocytic receptor for HA, capable of functioning independently of the 300 kDa HARE. Although it is possible that the 175 kDa HARE and 300 kDa HARE species could function together as a large complex, it is apparently not necessary for these two HAREs to be present in the same cell in order to create a specific functional HA receptor. Therefore, the 175 kDa HARE and 300 kDa HARE are independent isoreceptors. Studies are in progress to determine whether sinusoidal endothelial cells express either one of the HARE species alone or always together, and if the expression pattern of the two HARE species is tissue specific.

The results provided herein indicate that the native rat 175-kDa HARE protein is most likely derived from the proteolytic processing of a larger protein in LECs. Although this cannot be unequivocally proven until this larger protein is identified and shown to generate the 175-kDa HARE species, the following results indicate that the precursor protein is one of the two large subunits of the ˜300-kDa HARE. First, the 260 kDa and 230 kDa subunits of the ˜300-kDa HARE are immunologically related to the 175-kDa HARE, since they cross-react with all mAbs against the 175-kDa HARE. Second, the 175-kDa HARE does not have a unique N-terminus, indicating that it is sensitive to one or more cellular proteases. Third, the mRNA encoding the 175-kDa HARE is longer than expected for this size protein. Fourth, our present partial cDNA for the HARE protein encodes >200 amino acids upstream of the N-terminal Ser of the functional 175-kDa HARE. Finally, the two largest HARE proteins were reactive with an Ab against a predicted amino acid sequence upstream of the cDNA region encoding the native 175-kDa HARE. The latter result, in particular, strongly supports the proteolytic processing model. Therefore, the 260 kDa subunit (or its precursor) is the initial gene product, from which both the 230 kDa and 175-kDa proteins are then derived by proteolysis.

The rat 175 kDa HARE protein identified herein is a functional endocytic HA receptor when expressed from a non-naturally occurring synthetic cDNA. The protein is not directly encoded by an mRNA, but rather is apparently derived from the proteolytic processing of a larger protein, which may be the large 260 kDa subunit of the 300 kDa HARE complex. The mRNA encoding the rat 175 kDa HARE is ˜10 kb, substantially longer than that required for this size protein. That the characteristics of the rat and human HAREs are similar indicates a similar proteolytic processing may generate the human 190 kDa HARE from one of the large subunits of the 315 kDa HARE. The two human HARE isoreceptors described here have a very similar organization to the two rat HAREs, and the three anti-rat mAbs that recognize the 190 kDa human HARE also cross-react with the two large subunits of the human ˜315 kDa HARE.

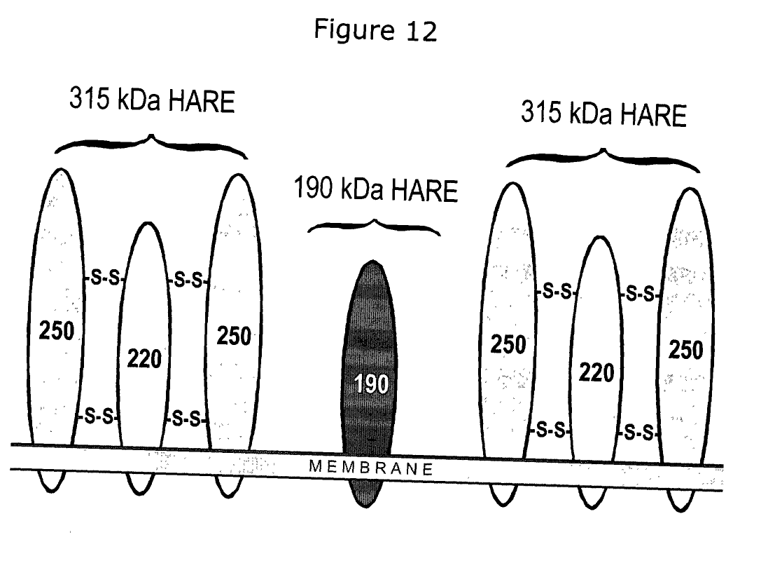

The organization of the two HARE isoreceptors purified from human spleen is depicted in FIG. 12. The 190 kDa and ˜315 kDa HAREs are most likely isoreceptors able to function independently as coated pit mediated endocytic receptors for HA. The 190 kDa HARE contains a single protein. The ˜315 kDa HARE disulfide-bonded complex contains 2-3 copies of a 250 kDa subunit and 1 copy of a 220 kDa subunit. Spleen has approximately 2-3 times more of the ˜315 kDa HARE species compared to the 190 kDa HARE. It is proposed that the large HARE may be more effective in binding and in internalizing larger HA and the smaller HARE may be more effective in recognizing smaller HA. Since the size distribution of HA varies ˜100-fold in the body, more than one HARE may be needed physiologically to remove it.

The large extracellular domain of the 190 kDa HARE is predicted (Schultz et al, Proc. Natl. Acad. Sci. USA 95:5857 (1998)) to contain a delta serrate ligand (DSL) domain, and up to four β-Ig-H₃/fasciclin-like domains, three Metallothionein domains, four Furin-like domains, a Link domain and ˜24 EGF-like domains (many of which overlap) arranged in two cysteine-rich clusters separated by a 353 amino acid region that is cysteine-poor. Fasciclins are Ig-like cell adhesion molecules originally found on a subset in insects of axons during neuronal development (Kose et al, Development, 124:4143 (1997)). The EGF-like domains include laminin-like, EGF-1, EGF-2 and Ca⁺²-binding domains (Selander-Sunnerhagen et al, J. Biol. Chem. 267:19642 (1992)). We showed previously in rat LECs that HARE can function without any divalent cations including Ca⁺² (Yannariello-Brown et al, J. Cell Biochem. 48:73 (1992)). Several of the EGF-like domains in the human HARE have the characteristic pattern of six cysteines needed for the typical organization and folding of this domain (Selander-Sunnerhagen et al, J. Biol. Chem. 267:19642 (1992)).

The cytoplasmic domain of the human HARE (˜Y¹³⁴⁵-L¹⁴¹⁶) contains four tyrosine, seven serine and five threonine residues, although only residues S¹³⁶², S¹⁴⁰², T¹³⁸⁸ Y¹³⁸⁴ and Y¹³⁹⁶ are predicted (by NetPhos 2.0) to be phosphorylated. PEST motifs for rapid degradation, or consensus sequences for 0-glycosylation by GIcNAc are not present. As expected, the cytoplasmic domain contains several putative, candidate motifs for targeting the protein to clathrin coated pits. The sequence YSYFRI¹³⁵⁰ at the junction between the transmembrane and cytoplasmic domains, contains an interesting overlapping combination of two φXXB motifs, where φ is either tyrosine or phenylalanine, X can be any amino acid and B is a hydrophobic residue with a bulky side chain. The LDL, mannose and cation-dependent mannose 6-phosphate receptors, which are recycling endocytic receptors, are targeted to coated pits by very similar overlapping φXXB motifs (Meliman, Annu. Rev. Cell Biol. 12:575 (1996)). A third candidate φXXB motif is present at FQHF¹³⁶⁰.

The Link domain is clearly a good candidate for an HA-binding region but it is very likely that other, perhaps multiple, non-Link HA-binding domains are also present in the extracellular domain of HARE. Day, Jackson and colleagues have extensively investigated the structural requirements for HA-binding activity of Link domains from different proteins (Bajorath et al, J. Biol. Chem. 273:338 (1998); Kahmann et al, Structure Fold Des. 8:763 (2000); Banerji et al, Protein Expr. Purif. 14:371 (1998); Mahoney et al, J. Biol Chem. Published Apr. 3, 2001, JBC, online). In general, the affinities of these link domains is in the 10⁶ M⁻¹ range, which is not suitable for efficient receptor mediated endocytosis. Receptor-ligand complexes targeted to coated pits typically have K_{d} values in the nM range. ECM proteins containing Link domains can form stable multivalent networks with HA, although the binding affinity of individual HA-Link domain interactions is weak. Based on these above considerations, the extracellular domain of HARE contains multiple HA-binding regions. The formation of multivalent interactions of an HA molecule with several HA-binding domains on separate HAREs would not occur as efficiently as multiple interactions within the same HARE molecule. The longer ˜315 kDa HARE isoreceptor probably has more HA-binding domains than the smaller 190 kDa HARE.

The human HARE sequence reported here shares a high level of identity with a family of human proteins, as well as the rat 175 kDa HARE, shown in FIG. 7. One of these deduced human proteins, derived from accession number AAF82398, was designated FELL because it contains Fasciclin, EGF-Like, and Link domains. The three sequences represented by AAF82398, CAB61358 and BAB15793, have 95% identity among themselves and may be the same species; the slight differences could be due to sequencing errors or alternative splicing. The sequences of BAA13377 and CAB61827, which encodes stabilin-1, are more related to each other than to the three sequences noted above or to HARE. Although the BAA13377 mRNA sequence is present in endothelial cells, the presence of protein or associated HA-binding activity was not determined (Tsifrina et al, Am. J. Pathol. 155:1625 (1999)). Because we have identified the first function for a member of this protein family, it may be more relevant now to designate these proteins as HARE or HARE-like rather than FELLs.

The overall similarities in their extracellular, transmembrane and cytoplasmic domains suggest that the members of this HARE protein family may all be able to bind HA, chondroitin, chondroitin sulfate or other glycosaminoglycans and mediate their endocytosis through the clathrin-coated pit pathway. The differences in their membrane and cytoplasmic domains also raise the possibility that the members of this family could interact with different membrane or cytoplasmic regulatory factors and consequently process or route these bound ligands through different intracellular pathways.

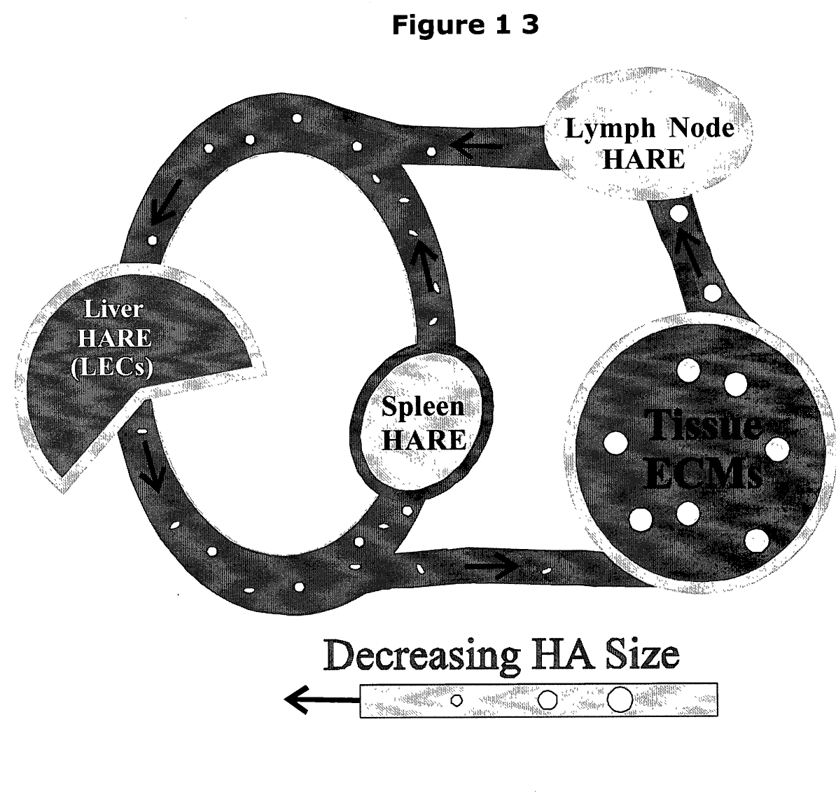

Our current model for HA turnover in mammals (FIG. 13) highlights the role of HARE in liver and lymph node and to a lesser extent in spleen. HARE mediates the uptake of HA into these tissues so it can be removed from the lymph or blood and degraded. A large fraction of the ˜5 g of HA turned over daily by humans is probably derived from skin, which contains about 50% of our total body HA (Abatangelo and Weigel, New Frontiers in Medical Sciences: Redefining Hyaluronan (2000); Laurent and Fraser, FASEB J. 6:2397 (1992)) and which remarkably, has a half-life of only ˜one day (Tammi et al, J. Invest. Dermatol. 97:126 (1991)). Presently there are important clinical uses for HA-containing devices in treating wounds and osteoarthritis and in eye surgery (Laurent and Fraser, FASEB J. 6:2397 (1992); Panay and Lower, Curr. Opin. Obstet. Gynecol. 11:379 (1999)). Additional future uses of HA in clinical applications are likely to be developed based on our growing understanding of the biology of HA and its multiple roles in wound healing (Iocona et al, J. Surg. Res. 76:111 (1998); Chen and Abatangelo, Wound Repair Regen. 7:79 (1999)), angiogenesis (West et al, Science, 14:1324 (1985); Deed et al, Int. J. Cancer, 71:251 (1997); Rahmanian et al, Exp. Cell Res. 237:223 (1997)), macrophage activation (Horton et al, J. Biol. Chem. 273:35088 (1998); Horton et al, Am. J. Physiol. Lung Cell Mol. Physiol. 279:707 (2000)) and metastasis (Csoka et al, Invasion Metastasis, 17:297 (1997); Delpech et al, J. Intern. Med. 242:41 (1997)). A variety of different drug delivery systems utilizing HA are also being developed (Cantor et al, Proc. Soc. Exp. Biol. Med. 217:471 (1998); Ilium et al, J. Control Release, 29:133 (1994); Luo et al, J. Control Release, 69:169 (2000)). Given the likely increase in the clinical uses of HA-containing devices and drugs it is important that we now understand the overall mechanism of HA turnover in the body. In particular, the present molecular identification and characterization of the human HA Receptor for Endocytosis responsible for HA clearance is timely and should facilitate further studies in this field.

The human gene encoding HARE, which is in the genome database (under accession # NT_{—}024383.2), is located on chromosome 12 and appears to be a highly fragmented and unusual gene. The HARE coding region for the 1416 amino acids reported here is present as about 37 exons, most of which are only 100-200 bp long, distributed relatively regularly over a ˜171 kb region. The mouse gene is similarly organized.

Thus it should be apparent that there has been provided in accordance with the present invention a purified nucleic acid segment having a coding region encoding functionally active HARE, methods of producing HARE from the HARE gene, methods of purifying HARE, and the use of fragments of HARE that specifically bind HA, chondroitin and chondroitin sulfate as well as antibodies directed thereto, that fully satisfies the objectives and advantages set forth above. Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

What is claimed is:

1. A method of targeting a compound to a tissue of an individual expressing a functionally active HARE, the method comprising the steps of:
providing at least one of an HA molecule, a chondroitin molecule and a chondroitin sulfate molecule;
providing a compound;
conjugating the compound to at least one of the HA molecule, the chondroitin molecule and the chondroitin sulfate molecule to form at least one of an HA-compound complex, a chondroitin-compound complex, and a chondroitin sulfate-compound complex, wherein a functionally active HARE selectively binds and endocytoses the HA-compound complex, the chondroitin-compound complex or the chondroitin sulfate-compound complex; and
administering an effective amount of at least one of the HA-compound complex, the chondroitin-compound complex and the chondroitin sulfate-compound complex to the individual.

2. A method of preventing interaction between a cell having at least one of an HA coat, a chondroitin coat and a chondroitin sulfate coat and a cell expressing HARE on a surface thereof, the method comprising the steps of:
providing a humanized monoclonal antibody that selectively binds to an epitope of HARE and inhibits the binding of at least one of HA, chondroitin and chondroitin sulfate to HARE;
administering an effective amount of the humanized monoclonal antibody, wherein the humanized monoclonal antibody selectively binds to the epitope of HARE expressed on the surface of cells and inhibits binding of at least one of HA, chondroitin and chondroitin sulfate in the coat of the cells to the cells expressing HARE.

3. A method of preventing interaction between a cell provided with at least one of an HA coat, a chondroitin coat and a chondroitin sulfate coat and a cell expressing HARE on a surface thereof, the method comprising the steps of:
providing a compound that inhibits binding of at least one of HA, chondroitin and chondroitin sulfate to HARE; and
administering an effective amount of the compound, wherein the compound inhibits binding of at least one of HA, chondroitin and chondroitin sulfate in the coat of the cells to the cells expressing HARE.

4. The method of claim 3 wherein the compound is a mimetic peptide that binds to HARE and inhibits the binding of at least one of HA, chondroitin and chondroitin sulfate to HARE.

5. The method of claim 3 wherein the compound is identified using an affinity matrix comprising an active peptide fragment of HARE bound to a solid support, such that by contacting the compound to the affinity matrix, binding of at least one of HA, chondroitin and chondroitin sulfate to the active peptide fragment of HARE of the affinity matrix is decreased.

6. The method of claim 5 wherein the active peptide fragment of HARE is a soluble fragment of HARE.

7. The method of claim 5 wherein the active peptide fragment of HARE is an extracellular domain of HARE.

8. The method of claim 5 wherein the active peptide fragment of HARE is selected from the group consisting of an HA-binding domain of HARE, a chondroitin-binding domain of HARE and a chondroitin sulfate-binding domain of HARE.

9. A method of targeting a compound to a cell of an individual wherein the cell does not express a functionally active HARE on a surface thereof, comprising the steps of:
providing at least one of a compound-HA conjugate, a compound-chondroitin conjugate and a compound-chondroitin sulfate conjugate;
providing a humanized monoclonal antibody that selectively binds to an epitope of HARE and inhibits binding of at least one of HA, chondroitin and chondroitin sulfate to HARE;
administering an effective amount of the humanized monoclonal antibody to the human patient such that the humanized monoclonal antibody binds HARE and blocks the binding of at least one of HA, chondroitin and chondroitin sulfate to HARE; and
administering an effective amount of at least one of the compound-HA conjugate, the compound-chondroitin conjugate and the compound-chondroitin sulfate conjugate to the human patient.

10. The method of claim 9 wherein the individual is a human.

11. The method of claim 9 wherein the cell that does not express a functionally active HARE on a surface thereof does express at least one cell surface or extracellular matrix component capable of binding at least one of HA, chondroitin and chondroitin sulfate.

12. A method of targeting a compound to a cell of an individual wherein the cell does not express a functionally active HARE on a surface thereof, comprising the steps of:
providing at least one of a compound-HA conjugate, a compound-chondroitin conjugate and a compound-chondroitin sulfate conjugate;
providing a compound that inhibits binding of at least one of HA, chondroitin and chondroitin sulfate to HARE;
administering an effective amount of the compound to the human patient such that the compound binds HARE and inhibits binding of at least one of HA, chondroitin and chondroitin sulfate to HARE; and
administering an effective amount of at least one of the compound-HA conjugate, the compound-chondroitin conjugate and the compound-chondroitin sulfate conjugate to the human patient.

13. The method of claim 12 wherein the individual is a human.

14. The method of claim 12 wherein the cell that does not express a functionally active HARE on a surface thereof does express at least one cell surface or extracellular matrix component capable of binding at least one of HA, chondroitin and chondroitin sulfate.

15. A method of targeting a compound to cells expressing HARE on a surface thereof in an individual, the method comprising the steps of:
providing a monoclonal antibody that selectively binds to an epitope of HARE;
providing a compound deleterious to cells in close proximity to the cells expressing HARE on a surface thereof upon delivery of the compound to the cells expressing HARE on a surface thereof;
conjugating the compound to the monoclonal antibody to provide a monoclonal antibody-compound conjugate; and
administering an effective amount of the monoclonal antibody-compound conjugate to the individual such that the monoclonal antibody selectively binds to cells expressing HARE on a surface thereof, thereby delivering the compound to the cells.

16. The method of claim 15 wherein, in the step of providing a compound, the compound is a chemotherapeutic agent.

17. The method of claim 15 wherein, in the step of providing a compound, the compound is a radioisotope.

18. The method of claim 15 wherein the individual is a human.

19. The method of claim 18 wherein, in the step of providing a monoclonal antibody, the monoclonal antibody is a humanized monoclonal antibody.

20. A method of targeting a compound to cells expressing HARE on a surface thereof in an individual, the method comprising the steps of:
providing a monoclonal antibody that selectively binds to an epitope of HARE;
providing a compound to be delivered to cells expressing HARE on a surface thereof;
conjugating the compound to the monoclonal antibody to provide a monoclonal antibody-compound conjugate; and
administering an effective amount of the monoclonal antibody-compound conjugate to the individual such that the monoclonal antibody selectively binds to cells expressing HARE on a surface thereof, thereby delivering the compound to the cells.

21. The method of claim 20 wherein the individual is a human.

22. The method of claim 21 wherein, in the step of providing a monoclonal antibody, the monoclonal antibody is a humanized monoclonal antibody.

23. A HARE-like protein, comprising:
a LINK domain;
at least one of a motif selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and sequences that are substantially identical to or only contain conserved or semi-conserved amino acid substitutions to SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18; and
wherein the HARE-like protein is able to bind at least one of HA, chondroitin and chondroitin sulfate.

24. The HARE-like protein of claim 23 wherein the HARE-like protein is able to endocytose at least one of HA, chondroitin and chondroitin sulfate.

25. A method of detecting at least one of HA, chondroitin and chondroitin sulfate in a sample, the method comprising the steps of:
providing a HARE protein or peptide fragment wherein the HARE protein or peptide fragment contains at least one of a HA-binding domain, a chondroitin-binding domain and a chondroitin sulfate-binding domain;
providing a sample;
contacting the sample with the HARE protein or peptide fragment to form a mixture wherein at least one of HA, chondroitin and chondroitin sulfate present in the sample is bound to the HARE protein or peptide fragment;
providing at least one of labeled HA, labeled chondroitin and labeled chondroitin sulfate;
contacting at least one of labeled HA, labeled chondroitin and labeled chondroitin sulfate with the mixture; and
determining that at least one of HA, chondroitin and chondroitin sulfate is present in the sample if the at least one of labeled HA, labeled chondroitin and labeled chondroitin sulfate does not bind or has decreased binding to the HARE protein or peptide fragment.

26. The method of claim 25 wherein, in the step of providing a sample, the sample is a biological fluid.

27. The method of claim 25, in the step of providing a HARE protein or peptide fragment, the HARE protein or peptide fragment is immobilized on a solid support.

28. A method of detecting at least one of HA, chondroitin and chondroitin sulfate in a sample, the method comprising the steps of:
providing a HARE protein or peptide fragment wherein the HARE protein or peptide fragment contains at least one of a HA-binding domain, a chondroitin-binding domain and a chondroitin sulfate-binding domain;
immobilizing the HARE protein or peptide fragment on a solid support; providing a sample;
contacting the sample with the immobilized HARE protein or peptide fragment wherein at least one of HA, chondroitin and chondroitin sulfate present in the sample is bound to the HARE protein or peptide fragment;
washing the immobilized HARE protein or peptide fragment to remove unbound sample;
providing a labeled HARE protein or peptide fragment wherein the HARE protein or peptide fragment contains at least one of a HA-binding domain, a chondroitin-binding domain and a chondroitin sulfate-binding domain;
contacting the labeled HARE protein or peptide fragment with the immobilized HARE protein or peptide fragment such that the labeled HARE protein or peptide fragment binds to the at least one of HA, chondroitin and chondroitin sulfate bound to the immobilized HARE protein or peptide fragment; and
determining that the sample contains at least one of HA, chondroitin and chondroitin sulfate when the labeled HARE protein or peptide fragment is detected on the immobilized HARE protein or peptide fragment.

29. The method of claim 28 wherein, in the step of providing a sample, the sample is a biological fluid.

30. A test kit for determining the presence of at least one of HA, chondroitin and/or chondroitin sulfate in a sample, comprising:
an immobilized HARE protein or an immobilized HARE peptide fragment that contains at least one of an HA-binding domain, a chondroitin-binding domain and a chondroitin sulfate-binding domain;
a labeled or tagged preparation of HA;
means for contacting a sample with a portion of the immobilized HARE protein or peptide fragment to form a mixture thereof; and
means for contacting the labeled or tagged preparation of HA with immobilized HARE protein or peptide fragment alone and with the mixture of sample and immobilized HARE protein or peptide fragment.

31. The test kit of claim 30 further including nonlabeled HA.

32. The test kit of claim 30 further including at least one specific glycosidase for identifying the particular glycosaminoglycans present among HA, chondroitin and chondroitin sulfate in the sample.

33. A test kit for determining the presence of at least one of HA, chondroitin and/or chondroitin sulfate in a sample, comprising:
an immobilized HARE protein or an immobilized HARE peptide fragment that contains at least one of an HA-binding domain, a chondroitin-binding domain and a chondroitin sulfate-binding domain;
a labeled or tagged preparation of HARE protein or HARE peptide fragment that contains at least one of an HA-binding domain, a chondroitin-binding domain and a chondroitin sulfate-binding domain;
means for contacting a sample with a portion of the immobilized HARE protein or peptide fragment to form a mixture thereof;
means for washing away unbound sample; and
means for contacting the labeled or tagged preparation of HARE protein or peptide fragment with at least one of HA, chondroitin and/or chondroitin sulfate bound to the immobilized HARE protein or peptide fragment.

34. The test kit of claim 33 further including at least one specific glycosidase for identifying the particular glycosaminoglycans present among HA, chondroitin and chondroitin sulfate in the sample.

35. A method of treating a disease in a patient, one symptom of which is an elevated level of at least one of HA, chondroitin and chondroitin sulfate in the blood or lymph, the method comprising the step of administering to the patient an effective amount of a vector encoding a functionally active HARE.

36. A method of treating a disease in a patient, one symptom of which is an elevated level of at least one of HA, chondroitin and chondroitin sulfate in the blood or lymph, the method comprising the step of administering to the patient an effective amount of a vector encoding a functionally active HARE-like protein, wherein the HARE-like protein comprises:
a LINK domain;
at least one of a motif selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, and sequences that are substantially identical to or only contain conserved or semi-conserved amino acid substitutions to SEQ ID NO:6, SEQ ID NO:7, SEQ ID NO:8, SEQ ID NO:9, SEQ ID NO:10, SEQ ID NO:11, SEQ ID NO:12, SEQ ID NO:13 SEQ ID NO:14, SEQ ID NO:15, SEQ ID NO:16, SEQ ID NO:17 and SEQ ID NO:18; and
wherein the HARE-like protein is able to bind at least one of HA, chondroitin and chondroitin sulfate and endocytose the at least one of HA, chondroitin and chondroitin sulfate.
